(19) United States
(12) Patent Application Publication
(10) Pub. No.: US 2025/0381153 A1
(43) Pub. Date: Dec. 18, 2025

(54) CURCUMIN COMPOUND COMPOSITION

(71) Applicants: UNIVERSITY OF LANCASHIRE, Preston, Lancashire (GB); Central University of Technology, Free State, Bloemfontein (ZA)

(72) Inventors: Chandrashekhar KULKARNI, Preston, Lancashire (GB); Sergey ZLATOGORSKY, Preston, Lancashire (GB); Pravin KENDREKAR, Preston, Lancashire (GB)

(73) Assignees: c/o UNIVERSITY OF LANCASHIRE, Preston, Lancashire (GB); Central University of Technology, Free State, Bloemfontein (ZA)

(21) Appl. No.: 19/110,013

(22) PCT Filed: Sep. 8, 2023
(86) PCT No.: PCT/GB2023/052326

§ 371 Date: Mar. 7, 2025

(30) Foreign Application Priority Data
Sep. 8, 2022 (GB) 2213122.1

(51) Int. Cl. A61K 31/12; A61K 9/06; A61K 9/107; A61K 9/1271; A61K 47/14; A61K 47/44

(52) U.S. Cl. CPC A61K 31/12; A61K 9/06; A61K 9/1075; A61K 9/1271; A61K 47/14; A61K 47/44

(57) ABSTRACT

The present invention relates to compositions, in particular to lipid-based compositions that solubilise and/or encapsulate (sometimes high concentrations of) curcumin (and/or derivatives or analogues thereof). Such lipid-based compositions offer a variety of uses, including in their capacity as topical or ingestible formulations. Such compositions permit delivery of high concentrations of curcumin (and its derivatives) in a highly bioavailable form.

### CROSS-REFERENCE TO RELATED APPLICATIONS

The present application is a U.S. National Stage Application under 35 U.S.C. § 371 of International Application No. PCT/GB2023/052326, filed on Sep. 8, 2023, which in turn claims the benefit of foreign priority under 35 U.S.C. § 119 from GB 2213122.1, filed on Sep. 8, 2022. The entire disclosure of the above applications is incorporated herein by reference.

### INTRODUCTION

The present invention relates to compositions, in particular to lipid-based compositions that solubilise and/or encapsulate (sometimes high concentrations of) curcumin (and/or derivatives or analogues thereof). The present invention also relates to methods of producing said compositions. The present invention also relates to various products that utilise or incorporate said compositions, and methods for making the same. Since the physical form of the compositions can be modulated, products containing or characterised by said compositions can enable a variety of modes of administration to and into the body. As such, the invention also pertains to various uses of said compositions and products, including medical uses.

### BACKGROUND

Curcumin is the main active ingredient present in turmeric used widely in Indian curries. It is a phenolic compound (chemically named as diferuloylmethane or [1,7-bis(4-hydroxy-3-methoxyphenyl)-1,6-heptadiene-3,5-dione]) primarily isolated from rhizomes of Curcuma longa. Evolving from the traditional medicine in India, it is under extensive investigation and development to treat various health ailments, such as biliary disorders, anorexia, cough, diabetic complications, hepatic disorders, rheumatism, and sinusitis—see Stohs, S. J.; Chen, O.; Ray, S. D.; Ji, J.; Bucci, L. R.; Preuss, H. G., Highly Bioavailable Forms of Curcumin and Promising Avenues for Curcumin-Based Research and Application: A Review. Molecules 2020, 25 (6), 1397. Reports on curcumin effectiveness over past five decades indicate that curcumin reduces blood cholesterol, prevents low-density lipoprotein oxidation, inhibits platelet aggregation, suppresses thrombosis and myocardial infarction (MI), suppresses symptoms associated with type 2 diabetes, rheumatoid arthritis, multiple sclerosis, Alzheimer's disease and Parkinson's disease, inhibits human immunodeficiency virus (HIV) replication, enhances wound healing, protects against liver injury, increases bile secretion, protects from cataract formation, and protects against pulmonary toxicity and fibrosis—see also Rakotoarisoa, M.; Angelova, A., Amphiphilic Nanocarrier Systems for Curcumin Delivery in Neurodegenerative Disorders. Medicines (Basel) 2018, 5 (4), 126.

Although curcumin has major health benefits, its applicability is hampered due innate concerns regarding its low bioavailability, poor solubility and limited stability—Beevers, C. S.; Huang, S., Pharmacological and clinical properties of curcumin. Botanics: Targets and Therapy 2011, 1, 5-18. Numerous attempts in past decade have tried to address these issues through the development and testing of various drug delivery nano-/micro-systems including vesicles/liposomes, solid lipid nanoparticles (SLN), nanostructured lipid carriers (NLC), polymer-based nanoparticles, mixed micelles, nanoemulsions and hydrogel or organogel complexes—see Rakotoarisoa et al Medicines (Basel) 2018; Rakotoarisoa, M.; Angelov, B.; Garamus, V. M.; Angelova, A., Curcumin- and Fish Oil-Loaded Spongosome and Cubosome Nanoparticles with Neuroprotective Potential against H2O2-Induced Oxidative Stress in Differentiated Human SH-SY5Y Cells. ACS Omega 2019, 4 (2), 3061-3073; Duan, Y.; Wang, J.; Yang, X.; Du, H.; Xi, Y.; Zhai, G., Curcumin-loaded mixed micelles: preparation, optimization, physicochemical properties and cytotoxicity in vitro. Drug Delivery 2015, 22 (1), 50-57; Hasan, M.; Belhaj, N.; Benachour, H.; Barberi-Heyob, M.; Kahn, C. J. F.; Jabbari, E.; Linder, M.; Arab-Tehrany, E., Liposome encapsulation of curcumin: Physico-chemical characterizations and effects on MCF7 cancer cell proliferation. International Journal of Pharmaceutics 2014, 461 (1), 519-528; Ahmed, K.; Li, Y.; McClements, D. J.; Xiao, H., Nanoemulsion- and emulsion-based delivery systems for curcumin: Encapsulation and release properties. Food Chemistry 2012, 132 (2), 799-807; Kharat, M.; McClements, D. J., Recent advances in colloidal delivery systems for nutraceuticals: A case study—Delivery by Design of curcumin. Journal of Colloid and Interface Science 2019, 557, 506-518. Some of these have shown to improve curcumin solubility and bioavailability, prevent degradation of curcumin by minimizing exposure to water, enhance permeability and elevated retention that allows targeting of tumour sites—Rakotoarisoa et al Medicines (Basel) 2018. These carrier systems were constructed from amphiphilic molecules, to aid in solubilizing polar (hydrophilic) and non-polar (hydrophobic) groups of curcumin. As a consequence, the possible degradation of curcumin in the gastrointestinal (GI) tract is also prevented—Hu, B.; Liu, X.; Zhang, C.; Zeng, X., Food macromolecule based nanodelivery systems for enhancing the bioavailability of polyphenols. Journal of Food and Drug Analysis 2017, 25 (1), 3-15.

In addition to the above, various other endeavours have sought to improve solubility and/or bioavailability (whilst preserving the stability curcumin), such as those by Esposito et al (Esposito, E.; Ravani, L.; Mariani, P.; Contado, C.; Drechsler, M.; Puglia, C.; Cortesi, R., Curcumin containing monoolein aqueous dispersions: A preformulative study. Materials Science and Engineering: C 2013, 33 (8), 4923-4934), Rachmawati et al (Rachmawati H, Budiputra D K, Mauludin R. Curcumin nanoemulsion for transdermal application: formulation and evaluation. Drug Dev Ind Pharm. 2015 April; 41(4):560-6. doi: 10.3109/03639045.2014.884127. Epub 2014 Feb. 7. PMID: 24502271), and Sanjeeb et al (WO2011101859A1). However, none of these endeavours led to a complete solution, for instance to the solving of the both bioavailability and solubility (particularly high loading of curcumin), and some prior art proposals use significant quantities of several ingredients, including polymers and/or surfactants, some of which are undesirable (or at least undesirable in the quantities used). Such problems are particularly pronounced for topical compositions, but also for oral dosage forms (including either solid or liquid forms, but especially solid).

It is an object of the present invention to provide alternative compositions, sometimes for alternative uses or in alternative products, to those described in the prior art.

It is an object of the present invention to provide compositions that permit relatively high loadings of curcumin (or its derivatives), especially where achievable without compromising bioavailability.

It is an object of the present invention to provide compositions that furnish curcumin (or its derivatives) in a bioavailable form, especially where achievable without having to reduce curcumin loading.

It is an object of the present invention to provide lipid-based compositions the afford better curcumin solubility and/or better curcumin encapsulation.

It is an object of the present invention to provide lipid-based compositions from which curcumin does not sediment after centrifugation—such compositions will suitable ensure curcumin is well encapsulated and/or thoroughly dissolved within lipids, thus providing a more consistent product.

It is an object of the present invention to provide lipid-based composition that utilise as fewer ingredients (or ingredient types/categories) as possible, especially to avoid certain types of surfactants and polymeric materials.

It is an object of the present invention to provide lipid-based compositions that may be adapted for a variety of uses.

It is an object of the present invention to provide lipid-based compositions that are stable (especially physically stable) for long periods upon storage.

It is an object of the present invention to provide lipid-based compositions in which curcumin remains chemically stable both during storage and during use (including upon the skin or within the body).

It is an object of the present invention to provide lipid-based compositions with high loadings of curcumin (or derivatives thereof) that can be readily dispersed (suitably after being stored) in water or other solvents, so as to produce oil-in-water compositions. Suitably such compositions would be stored in the absent (or with only low levels) of any surfactants.

It is an object of the present invention to provide a lipid-based system which is particularly optimised for curcumin formulation, storage, and delivery (especially delivery through the skin or orally/gastrointestinally).

### SUMMARY OF THE INVENTION

According to an aspect of the present invention, there is provided a composition, suitably a composition comprising a curcumin compound. The curcumin compound is suitably curcumin (or a derivative or an analogue thereof) or a pharmaceutically acceptable salt thereof. The composition is suitably a lipid-based composition comprising a curcumin compound and one or more lipids. Suitably said composition also comprises one or more glycerides, suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

In respect of the aforesaid aspects, suitably the or each glyceride (suitably each mono- and/or diglyceride) comprises one, two, or three fatty acid(s) (suitably respectively comprises one or two fatty acids in the case of mono- and/or diglyceride) condensed with glycerol. Suitably, the or at least one of, most suitably each (or all) of, said fatty acid(s) are C₆-C₃₀ fatty acid(s) (i.e. having a carbon chain that is 6-30 carbons in length, including the carboxylate carbon), preferably C₈-C₂₀ fatty acid(s), most preferably C₁₈ fatty acid(s). Preferably at least one (preferably each) of the fatty acid(s) comprise at least one unsaturated moiety, preferably at least one alkene moiety, most preferably at least one cis-alkene moiety. Most preferably, the fatty acid(s) is or comprises oleic acid, and most preferably the glyceride is (or the glycerides comprise) glyceryl monooleate (monoolein). In alternative embodiments, the fatty acid(s) is or comprises linoleic acid (suitably in addition to or instead of oleic acid), in which case most preferably the glyceride is (or the glycerides comprise) glyceryl monolinoleate (monolinolein) (suitably in addition to or instead of glyceryl monooleate).

The curcumin compound may be any suitable curcumin compound, though most preferably it is curcumin. The composition may comprise one or more curcumin compounds, though preferably at least one of said curcumin compounds (most preferably the main curcumin compound, i.e. that present at the highest wt %) is curcumin.

According to an aspect of the present invention, there is provided a composition comprising a curcumin compound and one or more monoglycerides, wherein at least one of the one or more monoglyceride(s) comprises at least one cis-alkene moiety.

According to an aspect of the present invention, there is provided a composition comprising a curcumin compound and one or more monoglycerides, wherein at least one of the one or more monoglyceride(s) comprises at least one cis-alkene moiety; on the proviso that the composition is not a food supplement, food additive, or a foodstuff.

According to an aspect of the present invention, there is provided a composition, suitably a composition comprising one or more curcumin compounds. Preferably, one of the curcumin compounds constitutes a “main” curcumin compound, typically referred herein to “a curcumin compound” or “the curcumin compound”. The main curcumin compound is preferably curcumin or a pharmaceutically acceptable salt thereof. The composition is suitably a lipid-based composition comprising the one or more curcumin compounds, including a main curcumin compound, and one or more lipids. Suitably said one or more lipids comprise or are one or more glycerides, suitably wherein at least one (preferably each) of the glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

According to an aspect of the present invention, there is provided a composition comprising:
a curcumin compound (or one or more curcumin compounds, suitably wherein one curcumin compound is a “main” curcumin compound); and
one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides;
wherein the curcumin compound is provided by a turmeric extract (and/or curcuminoid mixture) that is included within the composition;
wherein optionally the composition further comprises water, optionally in combination with a surfactant where the composition is a water-in-oil or oil-in-water dispersion or emulsion.

According to an aspect of the present invention, there is provided a composition comprising:
a curcumin compound (or one or more curcumin compounds, suitably wherein one curcumin compound is a “main” curcumin compound);
one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides; and
one or more oils, preferably one or more oils selected from the group consisting of moringa oil, coconut oil, sunflower oil, avocado oil, soya oil (or soybean oil), corn oil, or any combination thereof;
   wherein optionally the composition further comprises water, optionally in combination with a surfactant where the composition is a water-in-oil or oil-in-water dispersion or emulsion.

According to an aspect of the present invention, there is provided a composition comprising:
a curcumin compound (or one or more curcumin compounds, suitably wherein one curcumin compound is a “main” curcumin compound);
one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides; and
one or more oils, preferably one or more oils selected from the group consisting of moringa oil, coconut oil, sunflower oil, avocado oil, soya oil (or soybean oil), corn oil, or any combination thereof;
wherein the curcumin compound is provided by a turmeric extract (and/or curcuminoid mixture) that is included within the composition;
wherein optionally the composition further comprises water, optionally in combination with a surfactant where the composition is a water-in-oil or oil-in-water dispersion or emulsion.

Whilst the composition of the invention may be a foodstuff or a food additive, preferably the composition of the invention is not a foodstuff or a food additive. Preferably the composition is not a food supplement, food additive, or a foodstuff

According to an aspect of the present invention, there is provided a method of preparing a composition (suitably as defined herein), said method being suitably as defined herein. The method suitably comprises mixing a curcumin compound with the one or more glycerides.

According to an aspect of the present invention, there is provided a composition obtained by (or directly obtained by) the aforesaid method of preparing a composition.

According to an aspect of the present invention, there is provided a product comprising a composition (suitably as defined herein), said product being suitably as defined herein.

According to an aspect of the present invention, there is provided a method of preparing a product (suitably as defined herein), said method being suitably as defined herein. The method suitably comprises incorporating the composition within the product, suitably by mixing the composition with other components/ingredients of the product.

According to an aspect of the present invention, there is provided a product obtained by (or directly obtained by) the aforesaid method of preparing a product.

According to an aspect of the present invention, there is provided a composition for use as a medicament (or for use in treating a disease, disorder, or medical condition, suitably a disease, disorder, or medical condition as defined herein).

According to an aspect of the present invention, there is provided a use of a composition (suitably as defined herein, e.g. a pharmaceutical composition) or a product (suitably as defined herein, e.g. a pharmaceutical product) in the manufacture of a medicament (or in the manufacture of a medicament for treating a disease, disorder, or medical condition, suitably a disease, disorder, or medical condition as defined herein).

According to an aspect of the present invention, there is provided a method of treating a disease, disorder, or medical condition (suitably a disease, disorder, or medical condition as defined herein) in a subject (suitably in a subject identified as in need of treatment), the method comprising administering to the subject a therapeutically effective amount of a composition (suitably as defined herein, e.g. a pharmaceutical composition) or a product (suitably as defined herein, e.g. pharmaceutical product).

Any features, including optional, suitable, and preferred features, described in relation to any particular aspect of the invention may also be features, including optional, suitable and preferred features, of any other aspect of the present invention unless incompatible therewith.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention, and to show how embodiments of the same are put into effect, reference is now made, by way of example, to the following diagrammatic drawings, in which:

FIGS. 1A-D shows: A) various concentrations (1.2, 1.3, 1.5 and 2.0 mg/g, from left to right) of curcumin solubilized in molten DU; B) a schematic diagram depicting the likely solubilization/encapsulation of curcumin in DU, which forms liquid crystalline fluid lamellar (L_{α}) phase in dehydrated state, that melts into fluid isotropic (L₂) phase; C) Samples exhibiting encapsulation of curcumin (concentrations shown from L to R: 25, 30, 35, 40, 50, 60, 70 and 80 mg/g); and D) a histogram showing the curcumin concentrations in formulation F01 with error bars.

FIG. 2 A graph showing the intensity (I(q)) in arbitrary units as a function of scattering vector (q) in nm⁻¹. Patterns typically identify the presence of lamellar phase with and without curcumin using SAXS technique. Inset shows first order peaks of lamellar phase.

FIG. 3 shows DSC curves in heating-direction—showing curcumin effect in dry and wet states.

FIG. 4 shows the molecular structure of curcumin and the numbering scheme used in NMR characterisations.

FIG. 5 shows an assigned ¹H NMR spectrum (which was run on neat molten monoolein at 50° C. using the sealed d₆-dmso ((CD₃)₂SO) capillary for lock) and molecular structure of monoolein.

FIG. 6 shows an assigned ¹³C NMR spectrum (which was run on neat molten monoolein at 50° C. using the sealed d₆-dmso capillary for lock) and molecular structure of monoolein.

FIG. 7 shows a partially assigned ¹H NMR spectrum of the solution of curcumin (5 mg) in molten monoolein (1000 mg) at 50° C. (the sealed d₆-dmso capillary was used for lock).

FIG. 8 relates to Formulation F02 (curcumin in DU and water) and shows A) Various concentrations (from L to R 10.0, 7.5, 6, 5 and 4.0 mg/g) of curcumin solubilized in DU-water cubic phase; B) Schematic diagram depicting the 3-dimensional tortuous architecture (L) formed of lipid bilayers in bicontinuous cubic Pn3m phase (R); C) Encapsulation of curcumin (concentrations shown from L to R: i) 100, 200 mg/g and ii) 200, 400 and 500 mg/g); D) Histogram showing the curcumin concentrations in formulation F02 with error bars.

FIG. 9 relates to Formulation F03 (curcumin in nanostructured dispersion) and shows: A) Formulation 02 was ultrasonicated in presence of F127 solution, resulting into nanostructured dispersion (an emulsion in this case) After centrifugation, formulations containing >7.5 mg/g curcumin displayed a little sedimentation (as in ii)) but below this concentration formulation F03 were stable and homogeneous (as in i); B) Histogram showing the curcumin concentrations in formulation F03 with error bars.

FIG. 10 shows an emulsion stabilized by using non-PEG based surfactant-saponin.

FIG. 11 shows water-in-oil emulsions prepared using various lipid combinations. These can be loaded with curcumin to obtain creams/pastes of various viscosities as shown on the right.

FIG. 12 shows bile salt-lipid mixtures (Left and middle) and bile salt-lipid-water mixtures loaded with curcumin (right).

FIG. 13 shows Lipid formulations containing Moringa oil.

FIG. 13A shows a lipid formulation containing 0.7936 wt % curcumin, 49.96 wt % DU, and 49.96 wt % moringa oil (i.e. 50:50 DU/oil).

FIG. 13B shows a lipid formulation containing 0.7936 wt % curcumin, 89.28 wt % DU, and 9.92% moringa oil (i.e. 90:10 DU/oil).

FIG. 13C shows a lipid formulation containing 2.5341 wt % curcumin, 58.48 wt % DU, 6.498 wt % moringa oil, and 32.489 wt % water (i.e. 90:10 DU/oil).

FIG. 13D-1 shows a lipid formulation containing 0.397 wt % curcumin, 44.597 wt % DU, 4.9554 wt % moringa oil, 49.554 wt % water, and 0.49554 wt % Pluronic F127 surfactant (i.e. 90:10 DU:oil).

FIG. 13D-2 shows a lipid formulation containing 0.397 wt % curcumin, 39.64 wt % DU, 9.94 wt % moringa oil, 49.554 wt % water, and 0.4955 wt % F127 (i.e. 80:20 DU:oil).

FIG. 13D-3 shows a lipid formulation containing 0.397 wt % curcumin, 29.73% DY, 19.82% moringa oil, 49.55 wt % water, and 0.4955 wt % F127 (i.e. 60:40 DU:oil).

FIG. 14 shows lipid formulations containing DU:Coconut oil in the following proportions a) 90:10, b) 80:20 and c) 60:40. These samples contain curcumin in 2.5 mg/g of the total content [a) 900 mg DU+100 mg Coconut oil+1 g of 1% F127 solution in water; b) 800 mg DU+200 mg Coconut oil+1 g of 1% F127 solution in water; c] 600 mg DU+400 mg Coconut oil+1 g of 1% F127 solution in water]

FIG. 14A shows a lipid formulation containing 0.398 wt % curcumin, 49.8 wt % DU, and 49.8 wt % coconut oil (i.e. 50:50 DU/oil).

FIG. 14B shows a lipid formulation containing 0.596 wt % curcumin, 89.46 wt % DU, and 9.94% coconut oil (i.e. 90:10 DU/oil).

FIG. 14C shows a lipid formulation containing 1.96 wt % curcumin, 58.82 wt % DU, 6.535 wt % coconut oil, and 32.679 wt % water (i.e. 90:10 DU/oil).

FIG. 15 shows lipid formulations containing DU:Sunflower oil in the following proportions a) 90:10, b) 80:20 and c) 60:40. These samples contain curcumin in 2.5 mg/g of the total content [a) 900 mg DU+100 mg Sunflower oil+1 g of 1% F127 solution in water; b) 800 mg DU+200 mg Sunflower oil+1 g of 1% F127 solution in water; c]600 mg DU+400 mg Sunflower oil+1 g of 1% F127 solution in water].

FIG. 15A shows a lipid formulation containing 0.398 wt % curcumin, 49.8 wt % DU, and 49.8 wt % sunflower oil (i.e. 50:50 DU/oil).

FIG. 15B shows a lipid formulation containing 0.596 wt % curcumin, 89.46 wt % DU, and 9.94% sunflower oil (i.e. 90:10 DU/oil).

FIG. 15C shows a lipid formulation containing 1.96 wt % curcumin, 58.82 wt % DU, 6.535 wt % sunflower oil, and 32.679 wt % water (i.e. 90:10 DU/oil).

FIG. 16 shows lipid formulations containing DU:Avocado oil in the following proportions a) 90:10, b) 80:20 and c) 60:40. These samples contain curcumin in 2.5 mg/g of the total content [a) 900 mg DU+100 mg Avocado oil+1 g of 1% F127 solution in water; b) 800 mg DU+200 mg Avocado oil+1 g of 1% F127 solution in water; c] 600 mg DU+400 mg Avocado oil+1 g of 1% F127 solution in water]

FIG. 16A shows a lipid formulation containing 0.398 wt % curcumin, 49.8 wt % DU, and 49.8 wt % avocado oil (i.e. 50:50 DU/oil).

FIG. 16B shows a lipid formulation containing 0.596 wt % curcumin, 89.46 wt % DU, and 9.94% avocado oil (i.e. 90:10 DU/oil).

FIG. 16C shows a lipid formulation containing 1.96 wt % curcumin, 58.82 wt % DU, 6.535 wt % avocado oil, and 32.679 wt % water (i.e. 90:10 DU/oil).

FIG. 17 shows: a) Film mould to prepare curcumin loaded hydrogel-lipid films b) Drug release set-up containing curcumin loaded film c) released curcumin from hydrogel-lipid film.

FIG. 18 shows a lipid formulation containing 0.497 wt % curcumin, 49.75 wt % DU, and 49.75% soya oil.

FIG. 18A shows a lipid formulation containing 0.695 wt % curcumin, 89.37 wt % DU, and 9.93 wt % soya oil.

FIG. 18B shows a lipid formulation containing 2.344 wt % curcumin, 58.59 wt % DU, 6.51% soya oil, 32.55 wt % water.

FIG. 18C-1 shows a lipid formulation containing 0.347 wt % curcumin, 44.62 wt % DU, 4.958 wt % soya oil, 49.58 wt % water, 0.496 wt % F127.

FIG. 18C-2 shows a lipid formulation containing 0.347 wt % curcumin, 39.667 wt % DU, 9.916 wt % soya oil, 49.58 wt % water, 0.496 wt % F127.

FIG. 18C-3 shows a lipid formulation containing 0.347 wt % curcumin, 29.75 wt % DU, 19.83 wt % soya oil, 49.58 wt % water, 0.496 wt % F127.

FIG. 19 shows a lipid formulation containing 0.497 wt % curcumin, 49.75 wt % DU, and 49.75% corn oil.

FIG. 19A shows a lipid formulation containing 0.695 wt % curcumin, 89.37 wt % DU, and 9.93 wt % corn oil.

FIG. 19B shows a lipid formulation containing 2.344 wt % curcumin, 58.59 wt % DU, 6.51% corn oil, 32.55 wt % water.

FIG. 19C-1 shows a lipid formulation containing 0.347 wt % curcumin, 44.62 wt % DU, 4.958 wt % corn oil, 49.58 wt % water, 0.496 wt % F127.

FIG. 19C-2 shows a lipid formulation containing 0.347 wt % curcumin, 39.667 wt % DU, 9.916 wt % corn oil, 49.58 wt % water, 0.496 wt % F127.

FIG. 19C-3 shows a lipid formulation containing 0.347 wt % curcumin, 29.75 wt % DU, 19.83 wt % corn oil, 49.58 wt % water, 0.496 wt % F127.

FIG. 20 shows a lipid formulation containing 0.027 wt % curcumin, 99.3 wt % DU, 0.667 wt % other substances present in the turmeric.

FIG. 21 shows a lipid formulation containing 0.0895 wt % curcumin, 58.65 wt %, DU, 39.104 wt % water, 2.15 wt % other substances present in turmeric.

FIG. 22 shows a lipid formulation containing 1.88 wt % curcumin, 47.16 wt % DU, 47.16 wt % aloe vera gel, 3.77 wt % xanthan gum and vegetable glycerol.

FIG. 23 shows a lipid formulation containing 32.5 wt % curcumin, 50 wt % DU, 17.5 wt % others, where others includes of demethoxycurcumin, and bis-demethoxycurcumin.

FIG. 24 shows a lipid formulation containing 1.95 wt % curcumin, 50 wt % DU, 48.05 wt % others, where others is components of (Solgar) turmeric extract other than curcumin.

FIG. 25 shows a lipid formulation containing 2.5 wt % curcumin, 50 wt % DU, 47.5 wt % others, where others is the non-curcumin ingredients (demethoxycurcumin, bis-demethoxycurcumin, Vitamin D, polysorbate 80).

FIG. 26 shows a lipid formulation containing 21.66 wt % curcumin, 40 wt % DU, 26.6 wt % water, 11.6 wt % others, where others is primarily demethoxycurcumin, and bis-demethoxycurcumin.

FIG. 27 shows a lipid formulation containing 1.3 wt % curcumin, 40 wt % DU, 26.6 wt % water, 32.03 wt % others, where others includes components in turmeric extract other than curcumin.

FIG. 28 shows a lipid formulation containing 1.67 wt % curcumin, 40 wt % DU, 26.6 wt % water, 31.66 wt % others, where others includes demethoxycurcumin, bis-demethoxycurcumin, Vitamin D, polysorbate 80.

FIG. 29 shows a lipid formulation containing 1.89 wt % curcumin, 4.85 wt % DU, 92.23 wt % water, 1.02 wt % others, where others is primarily demethoxycurcumin, and bis-demethoxycurcumin.

FIG. 30 shows a lipid formulation containing 0.95 wt % curcumin, 4.85 wt % DU, 92.23 wt % water, 1.97 wt % others (i.e. components in turmeric extract except curcumin).

FIG. 31 shows a lipid formulation containing 1.114 wt % curcumin, 4.85 wt % DU, 92.23 wt % water, 2.799 wt % others (i.e. demethoxycurcumin, bis-demethoxycurcumin, Vitamin D, polysorbate 80).

### DETAILED DESCRIPTION OF THE INVENTION

### Definitions

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

Unless stated otherwise, any reference herein to an “average” value is intended to relate to the mean value.

Unless otherwise stated, wherever a claim or paragraph refers to another claim/paragraph “or any other claim/paragraph dependent thereon” (or similar such language), such “other claim/paragraph dependent thereon” includes other claims/paragraphs that are directly or indirectly (i.e. via one or more other dependencies) dependent thereon.

Herein, the term “compound of Formula [X]”, where [X] is a number, typically a roman numeral optionally followed by an alphanumeric character, may be abbreviated to “compound [X]”. A reference to either a “compound of Formula [X]” or “compound [X]” suitably includes a salt (e.g. pharmaceutically acceptable salt) or solvate (e.g. hydrate) thereof, and suitably also includes a synthetic equivalent thereof.

A “composition” typically comprises components and ingredients. Ingredients may also be referred to as components, and may be used interchangeably, especially in respect of compositions.

The term “ingredient” (or component) includes general ingredients (e.g. defined by reference to an ingredient class, for example, a sugar) or specific ingredients (e.g. sucrose, which is a specific ingredient from the sugar ingredient class).

Although not intended to be limiting, an ingredient may be defined by reference to a label, a name (especially where said name is well-known), a structure, a function, any other appropriate means, or any combination thereof. Where an ingredient (especially an ingredient class) is referred to by way of a functional label (e.g. a label or name which mentions a function or uses functional language, for example a “sugar stabiliser”), said ingredient may serve the stated function (e.g. in the context of a given composition), especially where said stated function can be readily verified by means stated within this specification. However, said functional label may be simply a label that demands no particular verification of the stated function, especially where this specification provides no particularly methods of verifying said function—in such cases the skilled person will readily discern the scope of the functionally-labelled ingredient by reference to other non-functional parts of the functional label and/or by reference to the context in which said functional label is applied, optionally by reference to any sub-definitions or definitions of other ingredients (or ingredient classes). As such, the term “sugar stabiliser” may simply mean a sugar, suitably a sugar as further defined by way of sub-definitions.

An ingredient (or component) may in itself be composed of (or comprise) a plurality of ingredients (e.g. sub-ingredients). For example, a lipid component may, unless stipulated to the contrary (e.g. “the lipid component is [a single lipid]”, or “the lipid component consists of [a specific lipid]”), comprise more than one lipid. As such, any general principles defined herein in respect of a composition (e.g. amounts; the notion of a composition consisting essentially of one or more components—this may equally apply to a component with sub-components/sub-ingredients) may also be applicable to a component capable itself of comprising a plurality of sub-components.

Where a composition is said to comprise a plurality of stipulated ingredients (optionally in stipulated amounts of concentrations), said composition may optionally include additional ingredients other than those stipulated. However, a composition said to comprise a plurality of stipulated ingredients may in fact consist essentially of or consist of all the stipulated ingredients, optionally in the amounts specified, optionally (where compatible with the applicable context) with a solvent system (e.g. water). In either circumstance, an individual component may itself comprise, consist essentially of, or consist of a sub-component or one or more sub-components. Herein, wherever the term “comprise” is used it may (whether in the context of a composition or component/ingredient thereof), where compatible within the applicable context, be replaced by “consists essentially of” or “consists of”.

Herein, unless incompatible with the context or unless stated otherwise, the term “stipulated” used in the context of a “stipulated [ingredient/component]” preferably means ingredients recited as being present within any given composition (or component).

Amounts given herein in respect of any given ingredient class will, unless specified to the contrary, be applicable to any specific ingredient within said ingredient class.

Herein, amounts stipulated for components and ingredients, whether specified in terms of “parts”, ppm (parts per million), percentages (%, e.g. wt %), or ratios, are intended to be by weight, unless stated otherwise.

“About” when used to modify a numerically defined parameter (e.g., a pH value) means that the parameter may vary, e.g., within the experimental accuracy of determining the parameter or by as much as much as 5% below or above the stated numerical value for that parameter, preferably by as much as 2% below or above the stated numerical value for that parameter. In preferred embodiments, a parameter described by the term “about” corresponds to the stated numerical value.

Herein, where a composition (or indeed a component that may contain sub-components) is said to “consists essentially of” particular component(s), said composition suitably comprises at least 70 wt % of said component(s), suitably at least 90 wt % thereof, suitably at least 95 wt % thereof, most suitably at least 99 wt % thereof. Suitably, a composition said to “consist essentially of” particular component(s) consists of said component(s) save for one or more trace impurities.

Where the quantity or concentration of a particular component of a given composition is specified as a weight percentage (wt % or % w/w), said weight percentage refers to the percentage of said component by weight relative to the total weight of the composition as a whole. It will be understood by those skilled in the art that the sum of weight percentages of all components of a composition will total 100 wt %. However, where not all components are listed (e.g. where compositions are said to “comprise” one or more particular components), the weight percentage balance may optionally be made up to 100 wt % by unspecified ingredients (e.g. a diluent, such as water, or other non-essentially but suitable additives). Most suitably, the sum of wt % of stipulated ingredients does not exceed 100 wt % and any combinations of wt % that would do so would by definition be excluded.

Herein, unless stated otherwise, the term “parts” (e.g. parts by weight, pbw) when used in relation to multiple ingredients/components, refers to relative ratios between said multiple ingredients/components. Expressing molar or weight ratios of two, three or more components gives rise to the same effect (e.g. a molar ratio of x, y, and z is x1:y1:z1 respectively, or a range x1-x2:y1-y2:z1-z2). Though in many embodiments the amounts of individual components within a composition may be given as a “wt %” value, in alternative embodiments any or all such wt % values may be converted to parts by weight (or relative ratios) to define a multi-component composition. This is so because the relative ratios between components is often more important than the absolute concentrations thereof in the liquid pharmaceutical compositions of the invention. Where a composition comprising multiple ingredients is described in terms of parts by weight alone (i.e. to indicate only relative ratios of ingredients), it is not necessary to stipulate the absolute amounts or concentrations of said ingredients (whether in toto or individually) because the advantages of the invention can stem from the relative ratios of the respective ingredients rather than their absolute quantities or concentrations. However, in certain embodiments, such compositions consists essentially of or consist of the stipulated ingredients and a diluents (e.g. water).

The term “mole percent” (i.e. mol %) is well understood by those skilled in the art, and the mol % of a particular constituent means the amount of the particular constituent (expressed in moles) divided by the total amount of all constituents (including the particular constituent) converted into a percentage (i.e. by multiplying by 100). The concept of mol % is directly related to mole fraction.

The term “substantially free”, when used in relation to a given component of a composition (e.g. “a composition substantially free of compound X”), refers to a composition to which essentially none of said component has been added. When a composition is “substantially free” of a given component, said composition suitably comprises no more than 1 wt %, suitably not more than 0.1 wt %, suitably no more than 0.01 wt %, suitably no more than 0.001 wt % of said component, suitably no more than 0.0001 wt % of said component, suitably no more than 0.00001 wt %, suitably no more than 0.000001 wt %, suitably no more than 0.0000001 wt % thereof, most suitably no more than 0.0001 parts per billion (by weight).

The term “free” or “entirely free”, when used in relation to a given component of a composition (e.g. “a composition entirely free of compound X”), refers to a composition containing none of said component.

Herein, unless incompatible in a given context, wherever a component is stipulated which is capable of ionization (e.g. protonation or deprotonation), the definition of said component preferably includes any suitable salts thereof, preferably pharmaceutically acceptable salts thereof. For example, this applies to any references herein to buffering agents (e.g. citric acid or citrate), amino acids, and such like. Likewise, unless incompatible in a given context, wherever a component is stipulated which is capable of neutralisation, the definition of said component preferably includes neutralised forms thereof—e.g. citric acid instead of citrate.

Herein, in the context of the present specification, a “strong acid” is suitably one having a pKa of −1.0 or less, whereas a “weak acid” is suitably one having a pKa of 2.0 or more. Herein, in the context of the present specification, a “strong base” is suitably one whose conjugate acid has a pKa of 12 or higher (suitably 14 or higher), whereas a “weak base” is suitably one whose conjugate acid has a pKa of 10 or less.

Unless stated otherwise, references herein to a “pKa” should be construed as a pKa value in water at standard ambient temperature and pressure (SATP), suitably of the conjugate acid of the relevant species.

Suitably, unless stated otherwise, where reference is made to a parameter (e.g. pH, pKa, etc.) or state of a material (e.g. liquid, gas, etc.) which may depend on pressure and/or temperature, suitably in the absence of further clarification such a reference refers to said parameter at standard ambient temperature and pressure (SATP). SATP is a temperature of 298.15 K (25° C., 77° F.) and an absolute pressure of 100 kPa (14.504 psi, 0.987 atm).

The invention concerns amongst other things the treatment of conditions treatable by curcumin compounds. The term “treatment”, and the therapies encompassed by this invention, include the following and combinations thereof: (1) inhibiting, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “inhibit” includes reference to delaying, stopping, reducing the incidence of, reducing the risk of and/or reducing the severity of an event, state, disorder or condition. Inhibiting an event, state, disorder or condition may therefore include delaying or stopping initiation and/or progression of such, and reducing the risk of such occurring. The products/compositions of the disclosure may be used to inhibit events, disorders and/or conditions which are disclosed herein.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Routes of administration useful in the disclosed methods include but are not limited to topical, oral, parenteral intravenous, intraperitoneal (ip), rectal, ophthalmic, nasal, and transdermal.

Herein, unless stated otherwise, all chemical nomenclature may be defined in accordance with IUPAC definitions.

Herein, the term “hydrocarbon” is well understood in the art, and refers to compounds containing carbon and hydrogen only. The term “hydrocarbyl” general refers any aliphatic, acyclic, or cyclic (including aryl) hydrocarbon group, suitably with no heteroatoms. Such compounds include, inter alia, alkanes, alkenes, alkynes, arenes, and cyclic versions thereof. The term “hydrocarbon” anthracene, naphthalene, benzene, and/or derivatives thereof (e.g. toluene).

Herein, the term “carbocyclyl”, “carbocycle” or “carbocyclic” refers to a radical of a non-aromatic cyclic hydrocarbon group, generally having from 3 to 10 ring carbon atoms (i.e. (3-10C)carbocyclyl) and zero heteroatoms in the non-aromatic ring system. Suitably, carbocyclyl groups include (3-nC)cycloalkyl and (3-nC)cycloalkenyl. Exemplary embodiments include: cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptanyl, bicyclo[2.2.2]octanyl, and the like.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO2 groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
I) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered heteroaromatic ring containing 1, 2 or 3 ring heteroatoms; and
o) a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

Wherever groups with large carbon chains are disclosed (e.g. (1-12C)alkyl, (1-8C)alkenyl, etc.), such groups may optionally be shortened, for instance containing a between 1 and 5 carbons (e.g. (1-5C)alkyl or (1-5C)alkenyl), or contain between 1 and 3 carbons (e.g. (1-3C)alkyl or (1-3C)alkenyl instead of (1-12C)alkyl or (1-8C)alkenyl).

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess telomerase inhibitory activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula I may exhibit polymorphism, and that the invention encompasses all such forms.

Compounds may exist in a number of different tautomeric forms and references to compounds include all such forms. For the avoidance of doubt, where a compound can exist in one of several tautomeric forms, and only one is specifically described or shown, all others are nevertheless embraced by the definition of the compound. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

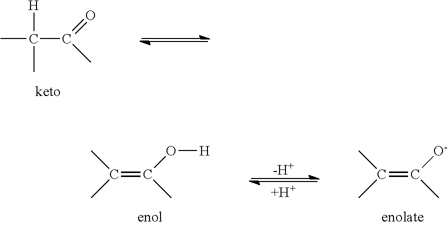

Herein, the term “particle size” or “pore size” refers respectively to the length of the longest dimension of a given particle or pore. Particle and pore sizes may be measured using methods well known in the art, including a laser particle size analyser and/or electron microscopes (e.g. transmission electron microscope, TEM, or scanning electron microscope, SEM).

The term “lipid” is well understood by a person skilled in the art, and generally hydrophobic or amphiphilic compounds and may inter alia include fats (incl. fatty acids, fatty acid esters, glycerides, fatty alcohols), waxes, sterols, fat-soluble vitamins (such as vitamins A, D, E, and K), monoglycerides, diglycerides, triglycerides, and phospholipids. Taking account of the biosynthesis of lipids and building blocks for their generation, e.g. ketoacyl or isoprene moieties, lipids may be categorised as fatty acids, glycerolipids, glycerophospholipids, sphingolipids, saccharolipids, and polyketides (the aforementioned being derivable through condensation of ketoacyl subunits); and sterol lipids and prenol lipids (which are derivable through condensation of isoprene subunits). The term lipid includes “glycerides” defined herein in relation to the invention. As such, references herein to lipids other than glycerides (e.g. in general or other than specific glycerides) generally refers to all of the above other than glycerides or the specific glycerides stipulated. Preferably, the term lipids encompasses only compounds comprising a fatty acid moiety (incl. fatty acids, fatty acid esters, glycerides, fatty alcohols), and, as such, lipids excluding glycerides (or specified glycerides) are compounds comprising a fatty acid moiety except for glycerides (or specified glycerides).

The term “oil” is well understood by a person skilled in the art, and refers to a hydrophobic, lipophilic, viscous liquid (at SATP) which generally comprises a mixture of compounds, suitably predominantly (≥50%, ≥60%, ≥70%, ≥80%, ≥90%, most suitably 95%) hydrophobic and lipophilic compound(s), suitably at least two or more of such compounds, suitably at least three or more such compounds, suitably at least four or more such compounds. Suitably all of the compound(s) present within any given oil are neutral (i.e. non-ionic and/or non-ionised). The or each oil suitably has a partition coefficient (octanol-Water partition coefficients, Log P) of at least 3, suitably at least 4, suitably at least 4.5, suitably at least 5, suitably at least 6. Suitably at least 70 wt % of the total weight of the or each oil comprises compounds which have a partition coefficient (octanol-Water partition coefficients, Log P) of at least 3, suitably at least 4, suitably at least 4.5, suitably at least 5, suitably at least 6, suitably at least 80 wt %, suitably at least 90 wt %, suitably at least 95 wt %. An “oil” tends to have a high carbon and high hydrogen content and are typically flammable. An “oil” is suitably surface active. An oil suitably has a particular viscosity measurable by techniques well known in the art. Herein, unless stated otherwise, all viscosity quantities given herein are kinematic viscosities at SATP, which is determinable by obtaining a dynamic viscosity (suitably at SATP, suitably at shear rates ranging from 0.1 sec⁻¹ to 100 sec⁻¹ or taking a dynamic viscosity as an average in these shear rate ranges) and converting to a kinematic viscosity in accordance with the following equation:

$$ν = μ / ρ \qquad ( 1 )$$

where ν is kinematic viscosity, μ is dynamic viscosity, and ρ is density of the overall fluid. Kinematic viscosities are expressed in centistokes (cSt), though SI units for kinematic viscosity are m²/s. Water at 20° C. has a kinematic viscosity of approximately 10⁻⁶ m²·s⁻¹ or 1 cSt. Suitably, the oil component has a kinematic viscosity between 10 and 500 cSt at SATP, suitably between 20 and 250 cSt, suitably between 30 and 150 cSt, suitably between 40 and 100 cSt, suitably between 50 and 80 cSt at SATP. Oils suitably comprise lipids (suitably at least 90 wt % lipids, suitably at least 95 wt % lipids, suitably at least 99 wt % lipids). Suitably, any amounts of oils is in addition to any amounts of stipulated components (e.g. specific glycerides) that may happen also to be present within the oils as well. Organic oils are generally derived from animal sources, plant sources, and other organisms—e.g. animal oils, vegetable oils, essential oils (which are generally from plants). Such organic oils are often produced through natural metabolic processes within such organisms. Where organic oils are used in the context of the present invention, suitably such organic oils exclude plant-derived essential oils. Organic oils generally comprise a mixture of lipids, and may optionally further include one or more substances selected from the group consisting of proteins, waxes, and alkaloids. The mixture of lipids may comprise compounds in one or more compound classes selected from the group consisting of fatty acids, glycerolipids, sterol lipids, steroids, phospholipids, sphingolipids, glycerophospholipids, prenol lipids, saccharolipids, polyketides, and optionally esters of any of the aforementioned.

References to fatty acids of glycerides refers to the fatty acyl moiety (i.e. the fatty acid that is condensed to the hydroxyl of glycerol).

The chemical structure of curcumin is:

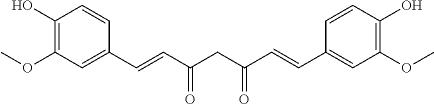

or its enol form:

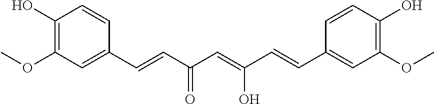

In general, references herein to either the β-diketone or enol tautomeric form of curcumin (or indeed curcumin compounds more generally), be it by way of words or chemical structures, also refers to the other tautomeric form. In general, the tautomeric forms are in equilibrium and may manifest in different proportions dependent on prevailing conditions—for instance, different solvents may affect the equilibrium concentration of the respect tautomers.

### General Points and Advantages Regarding the Invention

The present invention provides lipid-based compositions of curcumin compounds that are capable of a variety of applications. Some of the compositions are themselves products which may have a direct use—e.g. topical compositions, such as creams, ointments, etc. Some of the compositions may be used to produce products—e.g. dispersions for oral administration, wound healing products (e.g. bandages, gauzes, etc. incorporated with the composition) or, again, topical compositions.

Compositions of the invention make a technical contribution over the prior art, and represent a particularly excellent formulation solution for curcumin compounds specifically. That the lipid-based compositions of the invention perform quite so well in the context of curcumin compounds specifically was considered surprising.

Compositions of the invention permit high loadings of curcumin compounds, generally owing to the high levels of solubilisation and encapsulation achievable. That such high loadings of curcumin compounds are achievable was considered surprising, in the context of dry, hydrated, and aqueous formulations. It is especially surprising that such high loadings can be achieved without compromising curcumin bioavailability or stability. It is thought that the surprisingly high levels of solubilisation and/or encapsulation provided by the lipid-based systems described herein facilitates both bioavailability and stability (for both low and high loadings). The lipid-based system both protects the curcumin compounds, during storage and during administration (e.g. topically or orally/gastrointesintally), and provides an excellent vehicle for its delivery and ultimate release in a biological context.

The effective solubilisation and/or encapsulation of curcumin compounds within the lipid-based systems described herein is demonstrated by the minimal (or absence of) sedimentation (e.g. of the curcumin compound) observed following centrifugation of compositions of the invention. Such minimal (or lack of) sedimentation also testifies to the physical consistency of such compositions.

The advantages of the invention can be enjoyed in variety of use cases, particularly in medical applications (e.g. as vehicles for the topical or oral delivery of curcumin compounds, or in wound healing). Compositions of the invention may also be further processed (e.g. by mixing with other ingredients/components) into downstream products (e.g. oral dispersions) yet still benefit from the long-term storability afforded by substantially solid forms of compositions of the invention.

The lipid-based compositions of the invention are generally stable (especially physically stable) for long periods upon storage. Said compositions also allow curcumin compounds to remain chemically stable both during storage and during use (including upon the skin or within the body). Again it is thought that the lipid-based systems described herein are complementary to curcumin compounds to a surprising extent (apparently showing a high degree of structural and energetic complementarity in terms of molecular arrangements) owing to the excellent degree of solubilisation and/or encapsulation afforded to the curcumin compounds. The ‘cis’ conformation at the unsaturation is responsible for a kink in a lipid molecule causing its asymmetric bending. This, surprisingly, creates an explicit arrangement that complements the spatial orientation of curcumin; thus, facilitating curcumin localisation in this region. Such solubilisation and/or encapsulation appears to be a thermodynamic and a kinetic and phenomenon, respectively. Solubilisation preferably occurs in thermodynamically stable liquid crystalline systems, whereas encapsulation involves systematic contribution of hydrophobic forces, van Der Waals interactions and hydrogen-bonding along with the host lipid system's viscoelastic properties.

The lipid-based compositions of the invention perform surprisingly well even in the absence of high levels of surfactant(s). This is even so in aqueous dispersions of the compositions.

Lipid components employed in many formulations of the invention are common digestion products of consumed food fats (typically, triglycerides). Thus, lipid components herein are familiar to the internal milieu of the body which has a set mechanism for their breakdown and absorption in the bloodstream. This may facilitate a high degree of bioavailability of curcumin compounds.

### Composition

According to the invention, there is provided a composition. The composition may be as defined anywhere herein, including in paragraphs A1-A131, paragraphs B1-B450, or a combination thereof (since, as explained below, features of A1-A131 and B1-B450 may be combined by way of dependency or otherwise).

The composition suitably comprises curcumin compound, which is most preferably curcumin itself, though the curcumin compound may be curcumin (or a derivative, an analogue, or metabolite thereof) or a pharmaceutically acceptable salt thereof.

The composition is suitably a lipid-based composition comprising a curcumin compound and one or more lipids.

The composition suitably comprises one or more glycerides, suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

Whilst the composition of the invention may be a foodstuff or a food additive, preferably the composition of the invention is not a foodstuff or a food additive. Preferably, the composition is not a food supplement, food additive, or a foodstuff.

The lipid (glyceride)-based systems of the invention are particularly advantageous for formulating curcumin compounds for a variety of applications. Such advantages are thought to stem from a surprising degree of complementarity between their respective chemical structures and physical properties, which synergistically afford molecular arrangements maximising solubilisation and/or encapsulation of the curcumin compound(s) within the lipid (glyceride)-based system. It is thought that crystalline lamellar structures and/or fluid lamellar structures, coupled with a special capacity for curcumin compounds to bridge between individual lamella sheets and/or become encapsulated therein, may contribute to such advantages. Having unsaturated moieties, especially cis-alkene moieties, within relevant glyceride(s) is thought to be a key contributing factor in the particular case of curcumin compounds.

of curcumin compound(s) and the chemical structure and molecular arrangements adopted by the lipid (glyceride)-based systems in the presence of such curcumin compound(s).

### Type of Composition

In an embodiment, the composition is a topical composition (e.g. for use on a bodily surface, for example skin, eye(s), body cavity, and/or hair). Such a topical composition may be a composition as defined in any of paragraphs A1-A131, paragraphs B1-B450, or as defined anywhere else herein.

In an embodiment, the topical composition may be a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment. In a particular embodiment, the topical composition is a cream, an ointment, a foam, or a perfume (e.g. perfume, aftershave, cologne, or eau de toilette). In a particular embodiment, the topical composition is a cream. In another embodiment, the topical composition is an ointment. The present invention is particularly advantageous for topical compositions for the skin, permitting high concentrations of curcumin compounds to be rendered bioavailable for either local therapeutic effects or a more systemic therapeutic effect (e.g. following absorption through the skin). The invention also permits such topical compositions to exhibit a good “feel” on the skin.

In an alternative embodiment, the composition may be an oral topical composition, for example, a mouthwash, a rinse, an oral spray, a suspension, and/or a dental gel, suitably which is intended to be taken by mouth but are not intended to be ingested.

In an alternative embodiment, the composition is an ingestible composition (e.g. orally-administrable composition). Such an ingestible composition may be selected from a lozenge, a gel, a jelly, a pastille, a tablet, a capsule (e.g. a capsule containing the composition), a toffee, a nougat, a chewy candy, and/or a chewing gum. In a particular embodiment, the ingestible composition is (or is otherwise incorporated within) a pastille, a gel, or a jelly. In another embodiment, the ingestible composition comprises a gel component, gelling component, or gellable component. The gel component, gelling component, or gellable component is preferably animal- or plant-derived, most preferably plant-derived. In fact, most preferably, the composition or product into which said composition is incorporated is free of any animal-derived components/ingredients. The gel component, gelling component, or gellable component may comprise or consist of a polysaccharide or derivative thereof (e.g. x-carrageenan). The gel component, gelling component, or gellable component may comprise or consists of a gelatin, agar, and/or agarose. The ingestible composition may be (or may be otherwise incorporated within, for instance, by mixing the composition with a solvent such as water) a liquid, suitably a suspension, a dispersion, an emulsion, or a solution, most preferably a dispersion or emulsion. The ingestible composition may be a drink/beverage. The drink/beverage may be an alcoholic or non-alcoholic drink/beverage.

In an embodiment, the composition is (or is otherwise incorporated within) a pharmaceutical composition. Such a pharmaceutical composition is suitably for use as a medicament (or may otherwise be used in the manufacture of a medicament). Such a pharmaceutical composition may be (or may be otherwise incorporated within a product that is) an ayurvedic medicine. In an embodiment, the pharmaceutical composition is for (or is for use in) treating: neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukaemia, and colorectal cancer), anaphylaxis, arthritis, osteoarthritis, acne, and/or eczema.

The composition may suitably be (or may be otherwise incorporated within) a nutraceutical composition.

The composition may suitably be (or may be otherwise incorporated within) a food supplement.

The composition may suitably be (or may be otherwise incorporated within) a food additive.

The composition may suitably be (or may be otherwise incorporated within) a foodstuff.

Whilst the composition may be a food supplement, food additive, or a foodstuff, preferably the composition is not a food supplement, food additive, or a foodstuff.

The composition may be in the form of (or may be otherwise incorporated within) an emulsion, a lotion, a milk, a liquid, a solid, a cream, a gel, a mousse, an ointment, a paste, a serum, a stick, a spray, a tonic, an aerosol, a foam, and/or a pencil. The composition may be an oil-in-water dispersion. The composition may be a water-in-oil dispersion.

In an embodiment, the composition is (or is otherwise incorporated within) a personal care composition. Said personal care composition may be selected from the group consisting of skin care compositions, skin creams, shaving compositions (e.g. shaving creams, gels, foams), moisturizers, lotions, body washes, body oils, and hair care compositions (e.g. shampoos and/or hair conditioners). The personal care composition may be a “leave-on-type” composition (e.g. moisturizing lotions, serums, creams) or a “rinse-off-type” composition (e.g. body washes, shampoos, hair conditioners, shower gels, skin cleansers, cleansing milks, shaving compositions). The personal care composition may be in the form of (or may be otherwise incorporated within) an implement (e.g. a powdered or unpowdered cosmetic applicator), a reusable or disposable wipe, a tissue, a towel, a diaper, a razor or other shaving device, a personal cleansing implement (e.g. a mesh shower sponge), a conventional sponge, a wash cloth, a swab, and/or a pen.

In an embodiment, the composition is a cosmetic composition (or may be a cosmetic product or may be incorporated within a cosmetic product—e.g. a cosmetic product comprising the composition). The cosmetic composition may be for controlling and/or reducing the formation of wrinkles and lines in the skin. The cosmetic composition may be for providing glowing skin.

In an embodiment, the composition is a wound care composition (or may be a wound care product or may be incorporated within a wound care product—e.g. a wound care product comprising the composition). The wound care (or wound healing) composition may be a wound care hydrogel composition, and therefor suitably comprises a hydrogel or hydrogel-forming component or compound—this may be a polysaccharide-based hydrogel or hydrogel-forming component or compound. The wound care composition may be incorporated within a wound care product—e.g. a wound care product comprising the composition. Such a wound care product may be selected from the group consisting of a wound dressing, a sutures, a staple, a gauze, a bandage, a plaster, a burn dressing, an artificial skin, a liposome or micelle formulation, a microcapsule, an aqueous vehicle for soaking gauze dressings. In an embodiment, the wound care composition (or product) may be for (or for use in) healing wounds, for example, healing cuts and scrapes, burns, ulcers, bed sores, fissures, haemorrhoids, and/or post-surgical wounds. The same properties that render compositions of the invention excellent for topical applications also apply to wound care compositions, and products derived therefrom.

The composition may be (or may be otherwise incorporated within) a textile—e.g. a textile (e.g. a clothing item) comprising the composition.

### Curcumin Compound (Primary Active Ingredient)

The composition suitably comprises a curcumin compound. The curcumin compound is suitably curcumin (or a derivative or an analogue thereof) or a pharmaceutically acceptable salt thereof.

It will be understood that the composition may comprise one or more curcumin compound(s), as defined herein (i.e. extra curcumin compounds may fall within any definition set forth herein in relation not a “curcumin compound”), though preferably at least one of the one or more curcumin compound(s) is curcumin—NB. in general, unqualified references to “curcumin” includes curcumin or any pharmaceutically acceptable salt thereof. It will be understood by those skilled in the art that references herein to “a curcumin compound” or “the curcumin compound” preferably relates to a single (primary, or “main”) curcumin compound (most preferably curcumin itself), even though other curcumin compounds may also be present. Where one or more curcumin compounds are present within a composition, preferably curcumin is the main curcumin compound of the one or more curcumin compound(s) (i.e. the curcumin concentration, suitably as a wt % or ratio with any other component, is higher than any other curcumin compound). Suitably, the curcumin (or pharmaceutically acceptable salt thereof) constitutes between 30 wt % and 99.999 wt % of all curcumin compound(s) present within the composition. Suitably, the curcumin (or pharmaceutically acceptable salt thereof) constitutes between 30 wt % and 90 wt % of all curcumin compound(s) present within the composition. Where one or more curcumin compounds are present within a composition, preferably curcumin constitutes greater than or equal to 40 wt % of all curcumin compound(s) present, more preferably greater than or equal to 50 wt %, most preferably greater than or equal to 60 wt % of all curcumin compound(s) present. Where one or more curcumin compounds are present within a composition, the curcumin may constitute greater than or equal to 70 wt % of all curcumin compound(s) present. The curcumin may constitute greater than or equal to 80 wt % of all curcumin compound(s) present.

Compositions comprising one or more curcumin compounds may be defined as containing a main curcumin compound (preferably curcumin itself, or a pharmaceutically acceptable salt thereof) other curcumin compound(s). Suitably, the main curcumin compound may be present within the composition in an amount or ratio as defined herein in relation to “a curcumin compound” or “the curcumin compound” (which is preferably curcumin itself).

In an embodiment, the curcumin compound is defined by Formula I:

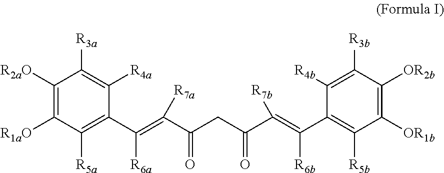

wherein:
   each R₁ₐ, R_{1b}, R₂ₐ, and R_{2b} group is independently selected from any R_{OX} group, and/or either or both pairs of R₁ₐ/R₂ₐ and/or R_{1b}/R_{2b} are linked together to form an optionally substituted heterocyclic or heteroaryl ring; and each R₃ₐ, R_{3b}, R₄ₐ, R_{4b}, R₅ₐ, R_{5b}, R₆ₐ, R_{6b}, R₇ₐ, and R_{7b} group is independently selected from any R_{CAR} group, and/or either or both pairs of R₃ₐ/R₄ₐ and/or R_{3b}/R_{4b} are linked together to form an optionally substituted cycloalkyl, cycloalkenyl, heterocyclic, or heteroaryl ring;
wherein:
each R_{OX} group is selected from hydrogen, trifluoromethyl, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N,N-di-[(1-6C)alkyl]sulphamoyl, or from a group of the formula:

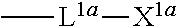

wherein:
   L^{1a} is absent or is selected from SO, SO₂, CO, CH(OR_{A}), CON(R_{A}), N(R_{A})CO, SO₂N(R_{A}), wherein R_{A} is hydrogen or (1-8C)alkyl; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{OX} group is optionally further substituted with one or more R_{CAR} groups as defined below; each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

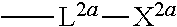

wherein:
   L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
   X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above;
wherein optionally either or both OR₁ₐ and/or OR_{1b} are replaced by hydrogen (e.g. dealkoxy analogues);
or any pharmaceutically acceptable salt thereof.

Suitably, each R_{OX} group is selected from hydrogen, (1-8C)alkyl, (2-6C)alkanoyl, wherein any R_{OX} group is optionally further substituted with one or more R_{CAR} groups.

Suitably, each R_{OX} group is selected from hydrogen and (1-3C)alkyl.

Suitably, each R_{OX} group is selected from hydrogen and methyl.

Suitably, both R₁ₐ and R_{1b} are the same as each other, and both R₂ₐ and R_{2b} are the same as each other, though suitably R₁ₐ/R_{1b} is different to R_{2a/2b}.

Suitably, both R₁ₐ and R_{1b} are methyl, and both R₂ₐ and R_{2b} are hydrogen.

In an embodiment, either or both OR₁ₐ and/or OR_{1b} are replaced by hydrogen (e.g. dealkoxy analogues).

Suitably, each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (1-8C)hydroxyalkyl, (1-6C)alkoxy, (1-6C)alkylamino, (1-6C)dialkylamino, (2-6C)alkanoyl, or from a group of the formula:

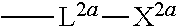

wherein:
   L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
   X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above.

Suitably, each R_{CAR} group is selected from hydrogen, halogeno, trifluoromethyl, cyano, hydroxy, amino, carboxy, (1-8C)alkyl, (1-8C)hydroxyalkyl, (1-6C)alkoxy, (2-6C)alkanoyl.

Suitably, each R_{CAR} group is selected from hydrogen, fluoro, or methyl.

Suitably, each R_{CAR} group is hydrogen.

Suitably, both R₃ₐ and R_{3b} are the same as each other, both R₄ₐ and R_{4b} are the same as each other, both R₅ₐ and R_{5b} are the same as each other, both R₆ₐ and R_{6b} are the same as each other, and both R₇ₐ and R_{7b} are the same as each other.

Suitably, each R₃ₐ, R_{3b}, R₄ₐ, R_{4b}, R₅ₐ, R_{5b}, R₆ₐ, R_{6b}, R₇ₐ, and R_{7b} group is hydrogen.

In an embodiment, the curcumin compound is defined by Formula II:

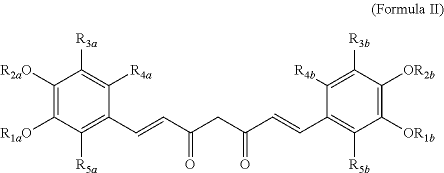

wherein R₁ₐ, R_{1b}, R₂ₐ, R_{2b}, R₃ₐ, R_{3b}, R₄ₐ, R_{4b}, R₅ₐ, and R_{5b}, are as defined anywhere herein;
wherein optionally either or both OR₁ₐ and/or OR_{1b} are replaced by hydrogen (e.g. dealkoxy analogues);
or any pharmaceutically acceptable salt thereof.

In an embodiment, the curcumin compound is defined by Formula III:

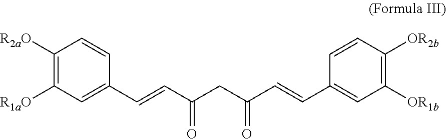

wherein R₁ₐ, R_{1b}, R₂ₐ, and R_{2b}, are as defined anywhere herein;
wherein optionally either or both OR₁ₐ and/or OR_{1b} are replaced by hydrogen (e.g. dealkoxy analogues);
or any pharmaceutically acceptable salt thereof.

In an embodiment, the curcumin compound is defined by Formula III:

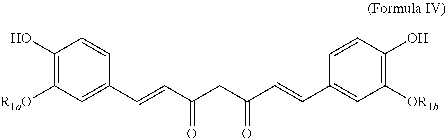

wherein R₁ₐ, and R_{1b}, are as defined anywhere herein;
wherein optionally either or both OR₁ₐ and/or OR_{1b} are replaced by hydrogen (e.g. dealkoxy analogues);
or any pharmaceutically acceptable salt thereof.

In an embodiment, the curcumin compound is defined by Formula IV:

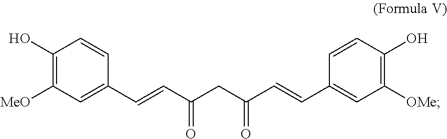 [SMILES: COc1cc(/C=C/C(=O)CC(=O)/C=C/c2ccc(O)c(OC)c2)ccc1O]

wherein optionally either or both OMe groups are replaced by hydrogen (e.g. dimethoxy or bisdemethoxy analogues); or any pharmaceutically acceptable salt thereof.

In an embodiment, the curcumin compound is curcumin or a pharmaceutically acceptable salt thereof.

In an embodiment, the curcumin compound is curcumin (suitably unsalted curcumin).

In an embodiment, the curcumin compound is a metabolite of curcumin, for example a curcumin glucuronide.

In an embodiment, the curcumin compound is demethoxycurcumin or a salt thereof (suitably unsalted demethoxycurcumin).

In an embodiment, the curcumin compound is bisdemethoxycurcumin or a salt thereof (suitably unsalted bisdemethoxycurcumin).

Suitably, the curcumin compound is substantially pure curcumin compound (e.g. greater than or equal to 95 wt % pure, suitably greater than or equal to 98 wt % pure, preferably greater than or equal to 99 wt % pure).

Suitably, the composition is substantially free of turmeric or comprises turmeric compounds (i.e. compounds within turmeric other than the curcumin compound) at a concentration of at most 5 wt %, suitably at most 2 wt %, preferably at most 1 wt %.

However, the curcumin compound (or “main” curcumin compound) may be provided to the composition as a constituent part of a curcumin compound source which comprises the (main) curcumin compound in admixture with one or more other compounds (e.g. one or more other curcumin compounds, and possibly other ingredients/components). The curcumin compound source may, for instance, be turmeric, a turmeric extract, and/or a curcuminoids mixture. Compositions formed using such a curcumin compound source may still furnish a required amount of the main curcumin compound (preferably curcumin itself), as defined anywhere herein. Any additional compounds within the curcumin compound source may be considered other source compounds.

For instance, the curcumin compound may be provided by turmeric or a turmeric extract, such that the composition comprises turmeric or a turmeric extract, a proportion of which is the curcumin compound. In such embodiments, the composition generally comprises one or more curcumin compounds (suitably with a “main” curcumin compound, as explained herein, along with other curcumin compound(s), e.g. curcuminoids). Compositions comprising turmeric or a turmeric extract may be defined as containing a (“main”) curcumin compound (preferably curcumin itself, or a pharmaceutically acceptable salt thereof) along with other turmeric compound(s) (which may include one or more other curcumin compounds, turmerones, and such like). However, compositions formed using turmeric or a turmeric extract may still furnish a required amount of the main curcumin compound (preferably curcumin itself), as defined anywhere herein. Any additional compounds within the turmeric or turmeric extract may be considered other turmeric compounds.

Whilst the present invention has been shown to be particularly applicable where the curcumin compound is curcumin perse, the complementarity between lipid-based systems of the invention and curcumin are credibly extrapolatable to all molecules bearing a curcumin or curcumin-like core, and particularly to those that exhibit extremely similar steric and electronic properties.

Suitably, the composition comprises 0.001-50 wt % curcumin compound. Suitably, the composition comprises 0.01-40 wt % curcumin compound. Suitably, the composition comprises 0.1-30 wt % curcumin compound.

Suitably, the composition comprises 0.01-10 wt % curcumin compound. Suitably, the composition comprises 0.001-3 wt % curcumin compound. Suitably, the composition comprises 0.01-1 wt % curcumin compound. Suitably, the composition comprises 0.1-4 wt % curcumin compound. Suitably, the composition comprises 0.1-5 wt % curcumin compound.

Suitably, the composition comprises 0.001-2 wt % curcumin compound. Suitably, the composition comprises 0.005-1 wt % curcumin compound. Suitably, the composition comprises 0.01-0.1 wt % curcumin compound.

Suitably, the composition comprises 15-40 wt % curcumin compound.

Suitably, the composition comprises 0.1-1 wt % curcumin compound.

Suitably, the composition comprises 0.5-1.5 wt % curcumin compound

Suitably, the composition comprises 1-10 wt % curcumin compound. Suitably, the composition comprises 5-10 wt % curcumin compound.

Suitably, the composition comprises 0.1-3 wt % curcumin compound. Suitably, the composition comprises 0.5-1.5 wt % curcumin compound.

Suitably, the composition comprises 0.5-5 wt % curcumin compound. Suitably, the composition comprises 1-2 wt % curcumin compound.

Suitably, the composition comprises 10-30 wt % curcumin compound. Suitably, the composition comprises 17.5-22.5 wt % curcumin compound.

Suitably, the composition comprises 0.005-0.5 wt % curcumin compound. Suitably, the composition comprises 0.01-0.1 wt % curcumin compound.

Suitably, the composition comprises 0.1-3 wt % curcumin compound. Suitably, the composition comprises 0.5-1.7 wt % curcumin compound.

Suitably, the curcumin compound is present within the composition at a concentration between 0.5 and 800 mg/g (i.e. 0.05-80 wt % of the total weight of the composition). Suitably, the curcumin compound is present within the composition at a concentration between 10 and 500 mg/g.

Suitably, the curcumin compound is present within the composition at a concentration greater than equal to 3.5 mg/g (i.e. ≥0.35 wt % of the total weight of the composition).

Suitably, the curcumin compound is present within the composition at a concentration greater than equal to 0.1 mg/g. Suitably, the curcumin compound is present within the composition at a concentration greater than equal to 1 mg/g. Suitably, the curcumin compound is present within the composition at a concentration greater than equal to 5 mg/g. Suitably, the curcumin compound is present within the composition at a concentration greater than equal to 10 mg/g. Suitably, the curcumin compound is present within the composition at a concentration greater than equal to 15 mg/g. Suitably, the curcumin compound is present within the composition at a concentration greater than equal to 30 mg/g. Suitably, the curcumin compound is present within the composition at a concentration greater than equal to 60 mg/g. Suitably, the curcumin compound is present within the composition at a concentration greater than equal to 100 mg/g. Suitably, the curcumin compound is present within the composition at a concentration greater than equal to 300 mg/g.

Suitably, the curcumin compound is present within the composition at a concentration less than equal to 600 mg/g. Suitably, the curcumin compound is present within the composition at a concentration less than equal to 500 mg/g. Suitably, the curcumin compound is present within the composition at a concentration less than equal to 200 mg/g. Suitably, the curcumin compound is present within the composition at a concentration less than equal to 100 mg/g.

Suitably, the curcumin compound is present within the composition at a concentration between 2-8 mg/g. Suitably, the curcumin compound is present within the composition at a concentration between 3-7 mg/g. Suitably, the curcumin compound is present within the composition at a concentration between 4-6 mg/g.

Suitably, the curcumin compound is present within the composition at a concentration between 5-10 mg/g. Suitably, the curcumin compound is present within the composition at a concentration between 6.5-8.5 mg/g.

Suitably, the curcumin compound is present within the composition at a concentration between 5-30 mg/g. Suitably, the curcumin compound is present within the composition at a concentration between 10-20 mg/g.

Suitably, the curcumin compound is present within the composition at a concentration between 10-500 mg/g.

Suitably, the curcumin compound is present within the composition at a concentration between 11-19 mg/g.

Suitably, the curcumin compound is present within the composition at a concentration between 10-40 mg/g. Suitably, the curcumin compound is present within the composition at a concentration between 15-35 mg/g. Suitably, the curcumin compound is present within the composition at a concentration between 20-30 mg/g.

Suitably, the curcumin compound is present within the composition at a concentration between 30-500 mg/g. Suitably, the curcumin compound is present within the composition at a concentration between 60-500 mg/g.

Suitably, the curcumin compound is present within the composition at a concentration between 40-150 mg/g (i.e. 6-10 wt %). Suitably, the curcumin compound is present within the composition at a concentration between 60-100 mg/g (i.e. 6-10 wt %). Suitably, the curcumin compound is present within the composition at a concentration between 65-95 mg/g.

Suitably, the curcumin compound is present within the composition at a concentration between 100-700 mg/g. Suitably, the curcumin compound is present within the composition at a concentration between 200-600 mg/g. Suitably, the curcumin compound is present within the composition at a concentration between 300-500 mg/g. Suitably, the curcumin compound is present within the composition at a concentration between 350-450 mg/g.

### Glycerides

The composition suitably comprises one or more glycerides, suitably in addition to a curcumin compound (suitably as defined herein). As such, the composition suitably comprises a glyceride.

The composition suitably comprises a mono- and/or di-glyceride. Most preferably, the composition comprises a monoglyceride.

Suitably, the composition comprises two or more glycerides. As such, suitably the composition comprises a first glyceride and a second glyceride. The composition suitably comprises a first glyceride and a second glyceride in a weight ratio between 1:99 and 99:1. The composition suitably comprises a first glyceride and a second glyceride in a weight ratio between 1:99 and 90:10. The composition suitably comprises a first glyceride and a second glyceride in a weight ratio between 10:90 and 50:50. The composition suitably comprises a first glyceride and a second glyceride in a weight ratio between 20:80 and 40:60. The composition suitably comprises a first glyceride and a second glyceride in a weight ratio between 25:75 and 35:65. The composition suitably comprises a first glyceride and a second glyceride in a weight ratio between 27:73 and 30:70.

Preferably, the aforesaid glyceride(s) is/are mono- and/or di-glyceride(s), though most preferably the aforesaid glyceride(s) is/are mono-glyceride(s). Mono-glyceride(s) are thought to facilitate particularly complementary molecular arrangements for solubilising and/or encapsulating curcumin compounds, and the inventors have shown that higher glycerides tend to reduce such solubilisation/encapsulation capacities.

Suitably, at least one of the glyceride(s) has a melting point of 25-55° C. Suitably, at least two (preferably the two which are mentioned/defined) of the glyceride(s) have a melting point of 25-55° C. Suitably, each defined glyceride(s) each has a melting point of 25-55° C. Suitably, at least one of the glyceride(s) has a melting point of 30-45° C. Suitably at least two (preferably the two which are mentioned/defined) of the glyceride(s) have a melting point of 30-45° C. Suitably, each defined glyceride(s) each has a melting point of 30-45° C. Suitably, at least one of the glyceride(s) has a melting point of 34-41° C. Suitably, at least two (preferably the two which are mentioned/defined) of the glyceride(s) have a melting point of 34-41° C. Suitably, each defined glyceride(s) each has a melting point of 34-41° C.

Suitably, the, each, or at least one of the, glyceride(s) comprises one or more unsaturated moieties. Suitably, the, each, or at least one of the, glyceride(s) comprises between one and two unsaturated moieties. Suitably, the, each, or at least one of the, glyceride(s) comprises one (i.e. a single, one and only one) unsaturated moiety. Suitably, the, each, or at least one of the, glyceride(s) comprises two (i.e. two and only two) unsaturated moieties.

Suitably, the composition comprises two glycerides, a first glyceride with one (i.e. one and only one) unsaturated moiety and a second glyceride with two (i.e. two and only two) unsaturated moieties. Suitably the respective first and second glyceride may be present in one of the aforementioned weight ratios.

Suitably, the, each, or at least one of the, unsaturated moiety(ies) is/are an alkene moiety(ies).

Suitably, the, each, or at least one of the, alkene moiety(ies) is/are cis-alkene moiety(ies).

Suitably, the, each, or at least one of the, glyceride(s) comprises one, two, or three fatty acid(s) (suitably respectively comprises one or two fatty acids in the case of mono- and/or diglyceride, and suitably respectively comprises only one fatty acid in the case of mono-glycerides) condensed with glycerol.

Suitably, the, each, or at least one of the, fatty acid(s) is/are aliphatic fatty acids. Suitably, the, each, or at least one of the, fatty acid(s) is/are linear (i.e. unbranched) fatty acids

Suitably, the, each, or at least one of the, fatty acid(s) is/are C₆-C₃₀ fatty acid(s) (i.e. having a carbon chain that is 6-30 carbons in length, including the carboxylate carbon). Suitably, the, each, or at least one of the, fatty acid(s) is/are C₈-C₂₀ fatty acid(s). Suitably, the, each, or at least one of the, fatty acid(s) is/are C₁₈ fatty acid(s).

Suitably, the, each, or at least one of the, fatty acid(s) is/are aliphatic C₆-C₃₀ fatty acid(s) (i.e. having a carbon chain that is 6-30 carbons in length, including the carboxylate carbon). Suitably, the, each, or at least one of the, fatty acid(s) is/are aliphatic C₈-C₂₀ fatty acid(s). Suitably, the, each, or at least one of the, fatty acid(s) is/are aliphatic C₁₈ fatty acid(s).

Suitably, the, each, or at least one of the, fatty acid(s) is/are linear C₆-C₃₀ fatty acid(s) (i.e. having a carbon chain that is 6-30 carbons in length, including the carboxylate carbon). Suitably, the, each, or at least one of the, fatty acid(s) is/are linear C₈-C₂₀ fatty acid(s). Suitably, the, each, or at least one of the, fatty acid(s) is/are linear C₁₈ fatty acid(s).

Suitably, the, each, or at least one of the, fatty acid(s) comprises one or more unsaturated moieties. Suitably, the, each, or at least one of the, fatty acid(s) comprises between one and two unsaturated moieties. Suitably, the, each, or at least one of the, fatty acid(s) comprises one (i.e. a single, one and only one) unsaturated moiety. Suitably, the, each, or at least one of the, fatty acid(s) comprises two (i.e. two and only two) unsaturated moieties.

Suitably, the composition comprises a first glyceride comprising a fatty acid (suitably only a single fatty acid, as per a mono-glyceride) with one or more unsaturated moiety(ies), and a second glyceride comprising a fatty acid (suitably only a single fatty acid, as per a mono-glyceride) with one or more unsaturated moiety(ies).

Suitably, the composition comprises a first glyceride comprising a fatty acid (suitably only a single fatty acid, as per a mono-glyceride) with one (i.e. one and only one) unsaturated moiety, and a second glyceride comprising a fatty acid (suitably only a single fatty acid, as per a mono-glyceride) with two (i.e. two and only two) unsaturated moieties.

Suitably, the, each, or at least one of the, unsaturated moiety(ies) is/are an alkene moiety(ies). Suitably, the, each, or at least one of the, alkene moiety(ies) is/are cis-alkene moiety(ies).

Suitably, wherein the, each, or at least one of the, fatty acid(s) is oleic acid. Suitably, wherein the, each, or at least one of the, fatty acid(s) is linoleic acid.

Preferably, the composition comprises glyceryl monooleate (monoolein).

Suitably, the composition comprises glyceryl monolinoleate (monolinolein).

Preferably, the composition comprises glyceryl monooleate (monoolein) and glyceryl monolinoleate (monolinolein). Suitably, the composition comprises glyceryl monooleate and glyceryl monolinoleate in a weight ratio between 1:99 and 90:10. Suitably, the composition comprises glyceryl monooleate and glyceryl monolinoleate in a weight ratio between 10:90 and 50:50. Suitably, the composition comprises glyceryl monooleate and glyceryl monolinoleate in a weight ratio between 20:80 and 40:60. Suitably, the composition comprises glyceryl monooleate and glyceryl monolinoleate in a weight ratio between 25:75 and 35:65. Suitably, the composition comprises glyceryl monooleate and glyceryl monolinoleate in a weight ratio between 27:73 and 30:70.

The use in lipid-based systems of the invention of a second glyceride in addition to a first, especially where the first is glyceryl monooleate and the second is glyceryl monolinoleate, can significantly increase solubilisation and/or encapsulation capacity for curcumin compounds. In fact, a second glyceride (especially where it, like the first, contains one or more unsaturated moieties), such as glyceryl monolinoleate, can unlock previously unobtainable encapsulation potential to permit lipid-based systems to hold onto curcumin compound(s) way beyond their natural solubility limit, often even without risk of sedimentation upon storage and/or centrifugation (sedimentation is otherwise quite common for over-saturated lipid-based systems).

Preferably, the curcumin compound (or relevant sub-definition(s) thereof) is solubilised and/or encapsulated within the glyceride(s) (or relevant sub-definition(s) thereof). Preferably, the curcumin compound is dissolved within the glyceride(s). Suitably, the curcumin compound is encapsulated within the glyceride(s). Suitably, the curcumin compound is both dissolved within and encapsulated within the glyceride(s).

Suitably, the composition comprises lamella structures comprising both the curcumin compound and the glyceride(s). Lamella structures are especially predominant in dehydrated (or low water content) systems.

Suitably, the composition is non-transparent (i.e. is opaque) at SATP. This is generally due to a prevailing non-isotropic structure (e.g. lamella structure). Suitably, the composition is non-transparent (i.e. is opaque) at temperatures within +/−5° C. (preferably +/−1° C.) of the melting point of the either the composition or of the predominant lipid or glyceride(s). Suitably, the composition becomes transparent upon melting (e.g. when heated at least 5° C. above the melting point of either the composition or of the predominant lipid or glyceride(s)).

Suitably, the lamella structures are characterised by a liquid crystalline fluid lamellar (L_{α}) phase comprising both the curcumin compound and the glyceride(s).

Suitably, one or more entire molecules of the curcumin compound are contained within a single lamella that also contains the glyceride(s). Suitably, one or more molecules of the curcumin compound bridge between two separate lamellae which each contains the glyceride(s) (i.e. such molecules of the curcumin compound are only partially contained within a single lamella). Suitably, the composition comprises some lamella structures comprising both the curcumin compound and the glyceride(s). Curcumin molecule may also lie flat i.e. perpendicular to the x-y plane of the lamellar phase (where lamellae are stacked on top of each other along on Z-axis).

Suitably, the composition comprises both the curcumin compound and the glyceride(s) in a cubic phase, preferably a bicontinuous cubic Pn3m phase. Such cubic phases are especially predominant in hydrated (water-containing, or high water content, especially >20 wt %) systems.

Suitably, the composition is transparent at SATP but may have a little opacity generally due to a prevailing non-isotropic structure of the lamella structures. Suitably, the composition is or becomes transparent at 40° C. or higher, suitably at 15° C. or higher. This is suitably due to lamella structures being converted back into a bicontinuous cubic Pn3m phase, which is substantially isotropic. This transparent behaviour generally holds until very high curcumin contents are reached (e.g. at or above 200 mg/g curcumin compound, or at or above 200:1000 weight ratio of curcumin compound to glyceride(s)), at which point the compositions become more opaque due to the consequent hexagonal (H₂) or other anisotropic phase

Suitably, the curcumin compound (or relevant sub-definition(s) thereof) and the glyceride(s) (or relevant sub-definition(s) thereof) are present within the composition at a defined relative weight ratio. Such a ratio may apply regardless of the level of dilution of the composition as a whole.

Suitably, the weight ratio of the curcumin compound to the glyceride(s) is between 0.5:1000 and 800:1000 (i.e. 5:10000-8:10). Suitably, the weight ratio of the curcumin compound to the glyceride(s) is between 10:1000 and 500:1000. Suitably, the weight ratio of the curcumin compound to the glyceride(s) is between 30:1000 and 500:1000. Suitably, the weight ratio of the curcumin compound to the glyceride(s) is between 60:1000 and 500:1000.

Suitably, the weight ratio of the curcumin compound to the glyceride(s) is between 0.5:1000 and 300:1000. Suitably, the weight ratio of the curcumin compound to the glyceride(s) is between 0.5:1000 and 30:1000. Suitably, the weight ratio of the curcumin compound to the glyceride(s) is between 0.5:1000 and 20:1000. Suitably, the weight ratio of the curcumin compound to the glyceride(s) is between 5:1000 and 100:1000.

Suitably, the weight ratio of the curcumin compound to the glyceride(s) is between 2:1000 and 8:1000. Suitably, the weight ratio of the curcumin compound to the glyceride(s) is between 4:1000 and 6:1000.

Suitably, the weight ratio of the curcumin compound to the glyceride(s) is between 5:1000 and 10:1000. Suitably, the weight ratio of the curcumin compound to the glyceride(s) is between 6.5:1000 and 8.5:1000.

Suitably, the weight ratio of the curcumin compound to the glyceride(s) is between 5:1000 and 30:1000. Suitably, the weight ratio of the curcumin compound to the glyceride(s) is between 10:1000 and 20:1000. Suitably, the weight ratio of the curcumin compound to the glyceride(s) is between 11:1000 and 19:1000.

Suitably, the weight ratio of the curcumin compound to the glyceride(s) is between 10:1000 and 40:1000. Suitably, the weight ratio of the curcumin compound to the glyceride(s) is between 15:1000 and 35:1000. Suitably, the weight ratio of the curcumin compound to the glyceride(s) is between 20:1000 and 30:1000.

Suitably, the weight ratio of the curcumin compound to the glyceride(s) is between 40:1000 and 150:1000. Suitably, the weight ratio of the curcumin compound to the glyceride(s) is between 60:1000 and 100:1000. Suitably, the weight ratio of the curcumin compound to the glyceride(s) is between 65:1000 and 95:1000. These ratios are especially appropriate where the composition comprises two glyceride(s) (preferably MO and ML).

Suitably, the weight ratio of the curcumin compound to the glyceride(s) is between 100:1000 and 700:1000. Suitably, the weight ratio of the curcumin compound to the glyceride(s) is between 200:1000 and 600:1000. Suitably, the weight ratio of the curcumin compound to the glyceride(s) is between 300:1000 and 500:1000. Suitably, the weight ratio of the curcumin compound to the glyceride(s) is between 350:1000 and 450:1000. These ratios are especially appropriate where the composition comprises two glyceride(s) (preferably MO and ML) and moderate concentrations of water (e.g. 10-60 wt % water).

Suitably, the stipulated (i.e. the defined) glyceride(s) (or relevant sub-definition(s) thereof) are the predominant glyceride(s), and suitably the predominant lipid(s) (e.g. fatty-acid-containing compounds), within the composition. Suitably, all glyceride(s) (including predominant glyceride(s)) and lipids within the composition are collectively termed lipid compounds.

Suitably, the stipulated (i.e. the defined) glyceride(s) (or relevant sub-definition(s) thereof) constitute at least 50 wt % of the total amount of glyceride(s) present within the composition (i.e. the “50 wt %” is relative to total glyceride(s) rather than the composition as a whole), and preferably constitute at least 50 wt % of the total amount of lipid(s) present within the composition (i.e. the “50 wt %” is relative to total lipid(s) rather than the composition as a whole). All glyceride(s) (including predominant glyceride(s)) and other lipids within the composition are collectively termed lipid compounds or just “lipids”. Suitably, the stipulated (or defined) glyceride(s) constitute at least 70 wt % of the total amount of glyceride(s) present within the composition. Suitably, the stipulated (or defined) glyceride(s) constitute at least 85 wt % of the total amount of glyceride(s) present within the composition. Suitably, the stipulated (or defined) glyceride(s) constitute at least 89 wt % of the total amount of glyceride(s) present within the composition. Suitably, the stipulated (or defined) glyceride(s) constitute at least 95 wt % of the total amount of glyceride(s) present within the composition.

Suitably, the stipulated (or defined) glyceride(s) constitute at least 70 wt % of the total amount of lipids present within the composition. Suitably, the stipulated (or defined) glyceride(s) constitute at least 85 wt % of the total amount of lipids present within the composition. Suitably, the stipulated (or defined) glyceride(s) constitute at least 89 wt % of the total amount of lipids present within the composition. Suitably, the stipulated (or defined) glyceride(s) constitute at least 95 wt % of the total amount of lipids present within the composition.

Suitably, glyceride(s) (stipulated and/or unstipulated) constitute at least 70 wt % of the total amount oflipids present within the composition. Suitably, glyceride(s) (stipulated and/or unstipulated) constitute at least 85 wt % of the total amount of lipids present within the composition. Suitably, glyceride(s) (stipulated and/or unstipulated) constitute at least 95 wt % of the total amount of lipids present within the composition. Suitably, glyceride(s) (stipulated and/or unstipulated) constitute at least 99 wt % of the total amount of lipids present within the composition.

### Solvent

The composition may comprise a solvent (e.g. water). The solvent is preferably a polar solvent, more preferably a protic solvent, most preferably water. Preferably, the only solvent present within the composition is water (or else preferably solvents other than water constitute at most 1 wt % of the composition, preferably at most 0.1 wt %, most preferably at most 0.01 wt %). A certain quantity of water can be particularly advantageous for increasing the curcumin compound loading capacity within the lipid-based systems of the invention—such water can not only increase solubilisation but can, surprisingly, also increase encapsulation capacity.

Suitably, the composition comprises 0-99 wt % water. Suitably, the composition comprises 0.01-95 wt % water. Suitably, the composition comprises 1-97 wt % water.

Suitably, the composition comprises less than or equal to 80 wt % water (i.e. the composition has at most 80 wt % water, and may contain 0 wt % water). Suitably, the composition comprises less than or equal to 60 wt % water (i.e. the composition has at most 60 wt % water, and may contain 0 wt % water). Suitably, the composition comprises less than or equal to 40 wt % water (i.e. the composition has at most 40 wt % water, and may contain 0 wt % water).

Suitably, the composition comprises less than or equal to 5 wt % water (i.e. the composition has at most 5 wt % water, and may contain 0 wt % water). Suitably, the composition comprises less than or equal to 2 wt % water. Suitably, the composition comprises less than or equal to 1 wt % water. These are relatively unhydrated/dehydrated compositions.

Suitably, the composition comprises 10-60 wt % water (i.e. a hydrated composition with a moderate amount of water). Such compositions are especially advantageous for solubilising and encapsulating large amounts of curcumin compound(s). Suitably, the composition comprises 15-60 wt % water. Suitably, the composition comprises 20-45 wt % water. Suitably, the composition comprises 25-43 wt % water. Suitably, the composition comprises 32-41 wt % water. Suitably, these compositions (with a moderate amount of water) are formed by adding a required amount of water to an unhydrated/dehydrated composition (suitably with no more than 5 wt % water, such as a composition of B394-B399), and suitably thereafter mixing, suitably with sufficient heating to melt the unhydrated/dehydrated composition or at least the glyceride(s) thereof. Wherever a curcumin compound is to be added (e.g. to form a hydrated composition containing significant quantities of curcumin), suitably curcumin addition is performed by mixing additional curcumin with the unhydrated/dehydrated composition (preferably heated so as to be in a melted state) before the addition of water. Products are suitably stored at or below 20° C.

Suitably, the composition comprises 40-85 wt % water. Such compositions may suitable include a surfactant. Suitably, the composition comprises 75-85 wt % water. Such compositions may suitable include a surfactant.

Suitably, the composition comprises 85-99.9 wt % water (i.e. very hydrated, suitably a dispersion or emulsion). Suitably, the composition comprises 45-99 wt % water. Such compositions may suitable include a surfactant. Suitably, the composition comprises 70-98 wt % water. Suitably, the composition comprises 90-97 wt % water. Such very hydrated compositions may suitable include a surfactant. Such a very hydrated composition is suitably formed by adding a required amount of water to an unhydrated/dehydrated composition or a moderately hydrated composition (e.g. of B394-B405), and suitably thereafter mixing, suitably with sufficient heating to melt the unhydrated/dehydrated or moderately hydrated composition or at least the glyceride(s) thereof.

Additional Ingredient(s) and Limitations in relation thereto

The composition may comprise one or more additional ingredients, or else the composition may be otherwise characterised by limits in relation to (e.g. a maximum amount/concentration/ratio of) said one or more additional ingredients—as such the composition may comprise said additional ingredient(s), may be free of said additional ingredient(s), or may have a stipulated maximum quantity thereof.

The one or more additional ingredients may be ingredients selected from the group consisting of additional active(s), additional lipid(s) (e.g. fatty acid(s)), natural oil(s), bile salt(s) (or bile acid(s)), hydrophilic molecule(s), hydrophobic molecule(s), amphiphilic molecule(s), surfactant(s), or any combination thereof.

Suitably, save for additional active(s) or the curcumin compound, the composition consists of only GRAS grade components. Suitably the entire composition contains only GRAS grade components.

### Additional Lipids (or Glycerides)

The one or more additional ingredients may comprise one or more additional lipids.

The one or more additional lipids may comprise one or more glyceride(s) other than those stipulated (e.g. those stipulated above or in paragraphs B87-B144 below), suitably where said one or more glyceride(s) other than those stipulated may be referred to as “unstipulated” glyceride(s). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 50:50 (i.e. the weight ratio is less than or equal to 50:50). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 30:70 (i.e. the weight ratio is less than or equal to 30:70). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 20:80 (i.e. the weight ratio is less than or equal to 20:80). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 15:85 (i.e. the weight ratio is less than or equal to 15:85). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 10:90 (i.e. the weight ratio is less than or equal to 10:90). Suitably, the weight ratio of unstipulated glyceride(s) (e.g. those other than the glyceride(s) stipulated in B87-B144) to stipulated glyceride(s) (e.g. those stipulated in B87-B144) is between 0:100 to 5:95 (i.e. the weight ratio is less than or equal to 5:95).

The one or more additional lipids may comprise one or more lipid(s) other than stipulated glyceride(s) (e.g. those stipulated in B87-B144), suitably where said one or more lipid(s) other than those stipulated may be referred to as “unstipulated” lipid(s). Suitably, the weight ratio of unstipulated lipid(s) (e.g. lipid(s) other than the glyceride(s) stipulated in B87-B144) to stipulated glyceride(s) (e.g. those stipulated in B87-B144) is between 0:100 to 50:50 (i.e. the weight ratio is less than or equal to 50:50). Suitably, the weight ratio of unstipulated lipid(s) (e.g. lipid(s) other than the glyceride(s) stipulated in B87-B144) to stipulated glyceride(s) (e.g. those stipulated in B87-B144) is between 0:100 to 30:70 (i.e. the weight ratio is less than or equal to 30:70). Suitably, the weight ratio of unstipulated lipid(s) (e.g. lipid(s) those other than the glyceride(s) stipulated in B87-B144) to stipulated glyceride(s) (e.g. those stipulated in B87-B144) is between 0:100 to 20:80 (i.e. the weight ratio is less than or equal to 20:80). Suitably, the weight ratio of unstipulated lipid(s) (e.g. lipid(s) those other than the glyceride(s) stipulated in B87-B144) to stipulated glyceride(s) (e.g. those stipulated in B87-B144) is between 0:100 to 15:85 (i.e. the weight ratio is less than or equal to 15:85). Suitably, the weight ratio of unstipulated lipid(s) (e.g. lipid(s) other than the glyceride(s) stipulated in B87-B144) to stipulated glyceride(s) (e.g. those stipulated in B87-B144) is between 0:100 to 10:90 (i.e. the weight ratio is less than or equal to 10:90). Suitably, the weight ratio of unstipulated lipid(s) (e.g. lipid(s) other than the glyceride(s) stipulated in B87-B144) to stipulated glyceride(s) (e.g. those stipulated in B87-B144) is between 0:100 to 5:95 (i.e. the weight ratio is less than or equal to 5:95).

### Additional Active

The one or more additional ingredients may comprise an additional active ingredient (e.g. in addition to curcumin compound). Suitably, the additional active ingredient is other than a (different) curcumin compound. Suitably, the additional active ingredient is derived from or is a component part of turmeric. Suitably, the additional active ingredient is or comprises a turmerone. Suitably, the composition comprises one or more turmerones.

In a particular embodiment, however, the composition is free of any additional active ingredients. In a particular embodiment, the composition is free of turmerones or comprises no more than 10 wt % turmerones. In a particular embodiment, the composition is free of turmerones or comprises no more than 1 wt % turmerones. In a particular embodiment, the composition is free of turmerones or comprises no more than 0.1 wt % turmerones. In a particular embodiment, the composition is free of turmerones or comprises no more than 0.01 wt % turmerones.

### Additional Surfactant

The one or more additional ingredients may comprise a surfactant. Suitably, in the term “surfactant” excludes glyceride(s) or at least excludes any glyceride(s) defined/stipulated.

The surfactant may be a non-ionic surfactant. The non-ionic surfactant may be selected from the group consisting of: a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof. The non-ionic surfactant may be selected from the group consisting of: a fatty alcohol, a fatty alcohol ether, a fatty acid ester, a fatty acid amide, alpha-tocopherol, polyglycerol esters, sucrose esters, saponins (e.g. saponin), and any combination thereof.

Suitably, the surfactant is selected from the group consisting of: polyglycerol esters, sucrose esters, saponins (e.g. saponin), and any combination thereof. Suitably, the surfactant is a saponin (e.g. saponin).

Suitably, where the composition comprises a surfactant, it does so on the proviso that the surfactant is not a PEG-based surfactant (e.g. the surfactant does not comprise polyethylene glycol polymers or copolymeric blocks of polyethylene glycol). Suitably, where the composition comprises a surfactant, it does so on the proviso that the surfactant is not a poloxamer. Suitably, where the composition comprises a surfactant, it does so on the proviso that the surfactant is not poloxamer 407. Suitably, where the composition comprises a surfactant, it does so on the proviso that the surfactant is not polysorbate 20. Suitably, where the composition comprises a surfactant, it does so on the proviso that the surfactant is not PEG-40 castor oil (e.g. not Killiphor RH 40). It is preferable to exclude or at least minimise the aforesaid surfactants.

Where the composition comprises a surfactant, suitably the surfactant is present (suitably where the composition comprises greater than or equal to 45 wt % water) at a concentration of 0.001-10 wt % of the overall composition. Where the composition comprises a surfactant, suitably the surfactant is present (suitably where the composition comprises greater than or equal to 45 wt % water) at a concentration of 0.01-2 wt % of the overall composition. Where the composition comprises a surfactant, suitably the surfactant is present (suitably where the composition comprises greater than or equal to 45 wt % water) at a concentration of 0.1-1.5 wt % of the overall composition. Where the composition comprises a surfactant, suitably the surfactant is present (suitably where the composition comprises greater than or equal to 45 wt % water) at a concentration of 0.4-1 wt % of the overall composition.

Suitably, the composition comprises at most 1 wt % surfactant(s), suitably at most 1 wt % of any surfactants defined in any of B176-B186 (especially poloxamer, polysorbate, and PEG-40 castor oil surfactants).

Suitably, the composition comprises at most 0.1 wt % surfactant(s), suitably at most 0.1 wt % of any surfactants defined in any of B176-B186 (especially poloxamer, polysorbate, and PEG-40 castor oil surfactants).

Suitably, the composition comprises at most 0.01 wt % surfactant(s), suitably at most 0.01 wt % of any surfactants defined in any of B176-B186 (especially poloxamer, polysorbate, and PEG-40 castor oil surfactants).

Suitably, the composition comprises at most 0.001 wt % surfactant(s), suitably at most 0.001 wt % of any surfactants defined in any of B176-B186 (especially poloxamer, polysorbate, and PEG-40 castor oil surfactants).

Suitably, the composition is free of surfactants, suitably free of any surfactants defined in any of B176-B186 (especially poloxamer, polysorbate, and PEG-40 castor oil surfactants).

### Additional Oils

The one or more additional ingredients may comprise one or more oils. Such oil(s) may suitably comprise or consist of a natural oil. Where oils are present, most suitably the oils comprise or consist of an organic oil (suitably animal or vegetable oil). Suitably, the organic oil is a vegetable oil (usually rich in mono, di, and/or triglycerides) selected from the group consisting of (or any combination thereof):
Common vegetable oils: Coconut oil, Corn oil, Cottonseed oil, Olive oil, Palm oil, Peanut oil (Ground nut oil), Rapeseed oil (including Canola oil), Safflower oil, Sesame oil, Soybean oil (or soya oil), Sunflower oil;
Nut oils: Almond oil, Beech nut oil, Brazil nut oil, Cashew oil, Hazelnut oil, Macadamia oil, Mongongo nut oil, Pecan oil, Pine nut oil, Pistachio oil, Walnut oil;
Citrus oils: Grapefruit seed oil, Lemon oil, Orange oil,
Cucurbitaceae oils: Bitter gourd oil, Bottle gourd oil, Buffalo gourd oil, Butternut squash seed oil, Egusi seed oil, Pumpkin seed oil, Watermelon seed oil;
Food Supplement oils: Açai oil, Black seed oil, Blackcurrant seed oil, Borage seed oil, Evening primrose oil, Flaxseed oil; Other Edible oils: Amaranth oil, Apricot oil, Apple seed oil, Argan oil, Avocado oil, Babassu oil, Ben oil, Borneo tallow nut oil, Cape chestnut oil, Carob pod oil (Algaroba oil)Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil (pressed), Niger seed oil, Poppyseed oil, Nutmeg butter, Okra seed oil, Papaya seed oil, Perilla seed oil, Persimmon seed oil, Pequi oil, Pili nut oil, Pomegranate seed oil, Poppyseed oil, Pracaxi oil, Virgin pracaxi oil, Prune kernel oil, Quinoa oil, Ramtil oil, Rice bran oil, Royle oil, Shea nuts, Sacha inchi oil, Sapote oil, Seje oil, Shea butter, Taramira oil, Tea seed oil (Camellia oil), Thistle oil, Tigernut oil (or nut-sedge oil), Tobacco seed oil, Tomato seed oil, Wheat germ oil;
Multi-purpose oils: Castor oil, Coconut oil (copra oil), Colza oil, Corn oil, Cottonseed oil, False flax oil, Hemp oil, Mustard oil, Palm oil, Peanut oil, Radish oil, Rapeseed oil, Ramtil oil, Rice bran oil, Safflower oil, Salicornia oil, Soybean oil (or soya oil), Sunflower oil, Tigernut oil, Tung oil;
Inedible oils: Copaiba, Jatropha oil, Jojoba oil, Milk bush, Nahor oil, Paradise oil, Petroleum nut oil, Pongamia oil (also known as Honge oil);
Drying oils: Dammar oil, Linseed oil, Poppyseed oil, Stillingia oil, Tung oil, Vernonia oil; and
Miscellaneous vegetable oils: Amur cork tree fruit oil, Artichoke oil, Astrocaryum murumuru butter, Balanos oil, Bladderpod oil, Brucea javanica oil, Burdock oil (Bur oil), Buriti oil, Candlenut oil (Kukui nut oil), Carrot seed oil (pressed), Castor oil, Chaulmoogra oil, Crambe oil, Croton oil (tiglium oil), Cuphea oil, Cupuagu butter, Honesty oil, Illipe butter, Jojoba oil, Mango oil, Mowrah butter, Neem oil, Ojon oil, Passion fruit oil, Rose hip seed oil, Rubber seed oil, Sea buckthorn oil, Sea rocket seed oil, Snowball seed oil (Viburnum oil), Tall oil, Tamanu or foraha oil, Tonka bean oil (Cumaru oil), Tucuma butter, Ucuhuba seed oil, moringa oil.

Suitably, the organic oil is a moringa oil, coconut oil, sunflower oil, avocado oil, soya oil (or soybean oil), corn oil, or any combination thereof. As such, in a particular embodiment, the composition comprises a moringa oil, coconut oil, sunflower oil, avocado oil, soya oil (or soybean oil), corn oil, or any combination thereof.

Suitably, the organic oil is a moringa oil, coconut oil, sunflower oil, avocado oil, or any combination thereof. As such, in a particular embodiment, the composition comprises a moringa oil, coconut oil, sunflower oil, avocado oil, or any combination thereof.

The one or more oils may be a single oil.

Suitably, the composition comprises 0.1-70 wt % oil(s). Suitably, the composition comprises 1-60 wt % oil(s). Suitably, the composition comprises 2-50 wt % oil(s).

Suitably, the composition comprises 1-70 wt % oil(s). Suitably, the composition comprises 2-60 wt % oil(s). Suitably, the composition comprises 3-50 wt % oil(s).

Suitably, the composition comprises 1-30 wt % oil(s). Suitably, the composition comprises 2-20 wt % oil(s). Suitably, the composition comprises 3-10 wt % oil(s).

Suitably, the composition comprises 1-50 wt % oil(s). Suitably, the composition comprises 2-40 wt % oil(s). Suitably, the composition comprises 3-30 wt % oil(s).

Where oil(s) are present, suitably the weight ratio of stipulated glyceride(s) to oil(s) is 99:1 to 1:99. Where oil(s) are present, suitably the weight ratio of stipulated glyceride(s) to oil(s) is 99:1 to 30:70. Suitably, the weight ratio of stipulated glyceride(s) to oil(s) is 95:5 and 30:70. Suitably, the weight ratio of stipulated glyceride(s) to oil(s) is 95:5 to 50:40. Suitably, the weight ratio of stipulated glyceride(s) to oil(s) is 90:10 to 60:40. Suitably, the weight ratio of stipulated glyceride(s) to oil(s) is 90:10 to 50:50.

It follows from above that an aspect of the invention provides a composition comprising:
a curcumin compound (or one or more curcumin compounds, suitably wherein one curcumin compound is a “main” curcumin compound);
one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides; and
one or more oils, preferably one or more oils selected from the group consisting of moringa oil, coconut oil, sunflower oil, avocado oil, soya oil (or soybean oil), corn oil, or any combination thereof;
   wherein optionally the composition further comprises water, optionally in combination with a surfactant where the composition is a water-in-oil or oil-in-water dispersion or emulsion.

Some specific examples of this aspect are shown in the table below, with varying glyceride(s):oil ratios. In all examples a single oil is used.

| Single Oil | Dry Composition | Hydrated Composition | O/W Dispersion/Emuls |
|---|---|---|---|
| Moringa (Ex 11) | 0.7936 wt % curcumin | 2.5341 wt % curcumin | 0.397 wt % curcumin |
| | 49.96 wt % DU | 58.48 wt % DU | 44.597 wt % DU |
| | 49.96 wt % moringa oil | 6.498 wt % moringa | 4.9554 wt % moringa oil |
| | (i.e. 50:50 DU/oil). | oil, and | 49.554 wt % water |
| | 0.7936 wt % curcumin | 32.489 wt % water | 0.49554 wt % Pluronic F127 |
| | 89.28 wt % DU | (i.e. 90:10 DU/oil) | (i.e. 90:10 DU:oil). |
| | 9.92% moringa oil | | 0.397 wt % curcumin |
| | (i.e. 90:10 DU/oil) | | 39.64 wt % DU |
| | | | 9.94 wt % moringa oil |
| | | | 49.554 wt % water |
| | | | 0.4955 wt % F127 |
| | | | (i.e. 80:20 DU:oil). |
| | | | 0.397 wt % curcumin |
| | | | 29.73% DU |
| | | | 19.82% moringa oil |
| | | | 49.55 wt % water |
| | | | 0.4955 wt % F127 |
| | | | (i.e. 60:40 DU:oil). |
| Coconut (Ex 12) | 0.398 wt % curcumin | 1.96 wt % curcumin | 0.24% curcumin |
| | 49.8 wt % DU | 58.82 wt % DU | 44.8% DU |
| | 49.8 wt % coconut oil | 6.535 wt % coconut oil | 4.97% coconut oil |
| | (i.e. 50:50 DU/oil). | 32.679 wt % water | 49.50% water |
| | 0.596 wt % curcumin | (i.e. 90:10 DU/oil). | 0.49% F127 |
| | 89.46 wt % DU | | (i.e. 90:10 DU:oil) |
| | 9.94% coconut oil | | |
| | (i.e. 90:10 DU/oil). | | |
| Sunflower (Ex 13) | 0.398 wt % curcumin | 1.96 wt % curcumin | 3.47% curcumin |
| | 49.8 wt % DU | 58.82 wt % DU | 43.33% DU |
| | 49.8 wt % sunflower oil | 6.535 wt % sunflower oil | 4.82% sunflower oil |
| | (i.e. 50:50 DU/oil). | 32.679 wt % water | 47.90% water |
| | 0.596 wt % curcumin | | 0.48% F127 |
| | 89.46 wt % DU | | (90:10 DU/oil) |
| | 9.94% sunflower oil | | |
| | (i.e. 90:10 DU/oil). | | |
| Avocado (Ex 14) | 0.398 wt % curcumin | 1.96 wt % curcumin | 3.47% curcumin |
| | 49.8 wt % DU | 58.82 wt % DU | 43.33% DU |
| | 49.8 wt % avocado oil | 6.535 wt % avocado oil | 4.82% avocado oil |
| | (i.e. 50:50 DU/oil). | 32.679 wt % water | 47.90% water |
| | 0.596 wt % curcumin | (i.e. 90:10 DU/oil) | 0.48% F127 |
| | 89.46 wt % DU | | |
| | 9.94% avocado oil | | |
| | (i.e. 90:10 DU/oil). | | |
| Soya (Ex 16) | 0.497 wt % curcumin | 2.344 wt % curcumin | 0.347 wt % curcumin |
| | 49.75 wt % DU | 58.59 wt % DU | 44.62 wt % DU |
| | 49.75% soya oil | 6.51% soya oil | 4.958 wt % soya oil |
| | 0.695 wt % curcumin | 32.55 wt % water | 49.58 wt % water |
| | 89.37 wt % DU | (i.e. 90:10 DU/oil) | 0.496 wt % F127. |
| | 9.93 wt % soya oil | | 0.347 wt % curcumin |
| | | | 39.667 wt % DU |
| | | | 9.916 wt % soya oil |
| | | | 49.58 wt % water |
| | | | 0.496 wt % F127. |
| | | | 0.347 wt % curcumin |
| | | | 29.75 wt % DU |
| | | | 19.83 wt % soya oil |
| | | | 49.58 wt % water |
| | | | 0.496 wt % F127 |
| Corn (Ex 17) | 0.497 wt % curcumin | 2.344 wt % curcumin | 0.347 wt % curcumin |
| | 49.75 wt % DU | 58.59 wt % DU | 44.62 wt % DU |
| | 49.75% corn oil. | 6.51% corn oil | 4.958 wt % corn oil |
| | 0.695 wt % curcumin | 32.55 wt % water | 49.58 wt % water |
| | 89.37 wt % DU | | 0.496 wt % F127. |
| | 9.93 wt % corn oil | | 0.347 wt % curcumin |
| | | | 39.667 wt % DU |
| | | | 9.916 wt % corn oil |
| | | | 49.58 wt % water |
| | | | 0.496 wt % F127. |
| | | | 0.347 wt % curcumin |
| | | | 29.75 wt % DU |
| | | | 19.83 wt % corn oil |
| | | | 49.58 wt % water |
| | | | 0.496 wt % F127. |
| Summary | 0.39-0.8 wt % curcumin | 1.9-2.6 wt % curcumin | 0.2-0.4 wt % curcumin |
| Compositions | 99.2-99.61 wt % DU and oil | 31-33 wt % water | 47-51 wt % water |
| | combined (in 90:10 to 50:50 | 64.4-67.1 wt % DU and oil | 0.47-0.51 wt % F127 |
| | weight ratios DU/oil) | combined (in 90:10 to 60:40 | 48.09-52.33 wt % DU and oil |
| | 6-15:1000 curcumin/DU | weight ratios DU/oil) | combined (in 90:10 to 60:40 |
| | | 22-44:1000 curcumin/DU | weight ratios DU/oil) |
| | | | 5-15:1000 curcumin/DU |
| | | | OR |
| | | | 0.2-4 wt % curcumin |
| | | | 47-51 wt % water |
| | | | 0.47-0.51 wt % F127 |
| | | | 44.49-52.33 wt % DU and oil |
| | | | combined (in 90:10 to 60:40 |
| | | | weight ratios DU/oil) |
| | | | 5-250:1000 curcumin/DU |

In an embodiment, the composition comprises 0.001-50 wt % curcumin compound; one or more glyceride(s) in a weight ratio of the curcumin compound to the glyceride(s) between 0.5:1000 and 800:1000; and one or more oil(s) in a weight ratio of the glyceride(s) to the oil(s) between 99:1 and 30:70. In relation to this embodiment, suitably the composition comprises 0.1-70 wt % oil(s), suitably 1-60 wt % oil(s), suitably 2-50 wt % oil(s).

Dry: In an embodiment, the composition comprises 0.1-1 wt % curcumin compound; one or more glyceride(s) in a weight ratio of the curcumin compound to the glyceride(s) between 5:1000 and 30:1000; and one or more oil(s) in a weight ratio of the glyceride(s) to the oil(s) between 99:1 and 30:70; wherein the composition comprises less than or equal to 5 wt % water (preferably less than or equal to 1 wt % water). In an embodiment, the composition comprises 0.001-3 wt % curcumin compound; one or more glyceride(s) in a weight ratio of the curcumin compound to the glyceride(s) between 0.5:1000 and 30:1000; and one or more oil(s) in a weight ratio of the glyceride(s) to the oil(s) between 99:1 and 1:99; wherein the composition comprises less than or equal to 5 wt % water (preferably less than or equal to 1 wt % water). In an embodiment, the composition comprises 0.01-1 wt % curcumin compound; one or more glyceride(s) in a weight ratio of the curcumin compound to the glyceride(s) between 0.5:1000 and 20:1000; and one or more oil(s) in a weight ratio of the glyceride(s) to the oil(s) between 95:5 and 30:70; wherein the composition comprises less than or equal to 5 wt % water (preferably less than or equal to 1 wt % water). In relation to these embodiments, suitably the composition comprises 1-70 wt % oil(s). Suitably, the composition comprises 2-60 wt % oil(s). Suitably, the composition comprises 3-50 wt % oil(s).

Hydrated: In an embodiment, the composition comprises 0.5-5 wt % curcumin compound; one or more glyceride(s) in a weight ratio of the curcumin compound to the glyceride(s) between 10:1000 and 500:1000; and one or more oil(s) in a weight ratio of the glyceride(s) to the oil(s) between 99:1 and 30:70; wherein the composition comprises 10-60 wt % water. In an embodiment, the composition comprises 0.01-10 wt % curcumin compound; one or more glyceride(s) in a weight ratio of the curcumin compound to the glyceride(s) between 0.5:1000 and 800:1000; and one or more oil(s) in a weight ratio of the glyceride(s) to the oil(s) between 99:1 and 30:70; wherein the composition comprises 10-60 wt % water. In an embodiment, the composition comprises 0.1-5 wt % curcumin compound; one or more glyceride(s) in a weight ratio of the curcumin compound to the glyceride(s) between 5:1000 and 100:1000; and one or more oil(s) in a weight ratio of the glyceride(s) to the oil(s) between 99:1 and 30:70; wherein the composition comprises 10-60 wt % water. In relation to these embodiments, suitably the composition comprises 1-30 wt % oil(s). Suitably, the composition comprises 2-20 wt % oil(s). Suitably, the composition comprises 3-10 wt % oil(s).

Dispersion A: In an embodiment, the composition (suitably an oil-in-water or water-in-oil dispersion or emulsion) comprises 0.1-1 wt % curcumin compound; one or more glyceride(s) in a weight ratio of the curcumin compound to the glyceride(s) between 5:1000 and 30:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.001-10 wt % of the overall composition). In an embodiment, the composition (suitably an oil-in-water or water-in-oil dispersion or emulsion) comprises 0.001-3 wt % curcumin compound; one or more glyceride(s) in a weight ratio of the curcumin compound to the glyceride(s) between 0.5:1000 and 800:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.01-2 wt % of the overall composition). In an embodiment, the composition (suitably an oil-in-water or water-in-oil dispersion or emulsion) comprises 0.01-1 wt % curcumin compound; one or more glyceride(s) in a weight ratio of the curcumin compound to the glyceride(s) between 0.5:1000 and 30:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.01-2 wt % of the overall composition). In relation to these embodiments, suitably the composition comprises 1-50 wt % oil(s). Suitably, the composition comprises 2-40 wt % oil(s). Suitably, the composition comprises 3-30 wt % oil(s).

Dispersion B: In an embodiment, the composition (suitably an oil-in-water or water-in-oil dispersion or emulsion) comprises 0.1-5 wt % curcumin compound; one or more glyceride(s) in a weight ratio of the curcumin compound to the glyceride(s) between 0.5:1000 and 800:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.001-10 wt % of the overall composition). In an embodiment, the composition (suitably an oil-in-water or water-in-oil dispersion or emulsion) comprises 0.01-10 wt % curcumin compound; one or more glyceride(s) in a weight ratio of the curcumin compound to the glyceride(s) between 0.5:1000 and 800:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.01-2 wt % of the overall composition). In an embodiment, the composition (suitably an oil-in-water or water-in-oil dispersion or emulsion) comprises 0.1-4 wt % curcumin compound; one or more glyceride(s) in a weight ratio of the curcumin compound to the glyceride(s) between 0.5:1000 and 300:1000; and one or more oil(s) in a weight ratio of the glyceride(s) to the oil(s) between 99:1 and 30:70; wherein the composition comprises 45-99 wt % water and optionally a surfactant (suitably a surfactant at a concentration of 0.01-2 wt % of the overall composition). In relation to these embodiments, suitably the composition comprises 1-50 wt % oil(s). Suitably, the composition comprises 2-40 wt % oil(s). Suitably, the composition comprises 3-30 wt % oil(s).

Though the oil(s) may be any suitable oil(s), especially organic oil(s), in the aforesaid embodiments, preferably the one or more oil(s) are selected from the group consisting of a moringa oil, coconut oil, sunflower oil, avocado oil, soya oil (or soybean oil), corn oil, or any combination thereof, though most preferably the oil(s) are a single oil selected from any of the aforesaid.

In the aforesaid embodiments, the composition(s) may consist of or consist essentially of the stipulated ingredients.

It will be readily appreciated that any of the aforesaid embodiments (including the features thereof, especially where defined relatively broadly), with any of the aforesaid qualifications, may be further defined and/or sub-defined as set forth herein, in particular (and where compatible in the given context) as set forth in paragraphs B1-B450 below.

### Additional Bile Acids or Bile Salts

The one or more additional ingredients may comprise individual or mixtures of bile acids or bile salts. As such, in an embodiment, the composition comprises a mixture of bile salts (and/or bile acid(s)). Bile salts (and/or bile acid(s)) are particularly advantageous for digesting lipid-based systems of the invention in situ following absorption into the body or absorption upon/with a locality upon or within the body to which the composition is applied.

### Additional Aloe Vera

The one or more additional ingredients may comprise aloe vera, and may optionally in addition comprise a gum (preferably xanthan gum) and/or glycerol (preferably vegetable glycerol).

In an embodiment, the composition comprises 0.001-50 wt % curcumin compound; one or more glyceride(s) in a weight ratio of the curcumin compound to the glyceride(s) between 0.5:1000 and 800:1000; and aloe vera (e.g. aloe vera gel) in a weight ratio of the glyceride(s) to the aloe vera between 99:1 and 30:70. Such embodiments may also suitably comprise a gum (preferably xanthan gum) and glycerol, preferably in a weight ratio between 10:1 to 1:10 (more preferably between 5:1 and 1:1, most preferably about 3:1), wherein suitably the weight ratio of aloe vera to the combined weight of xanthan gum and glycerol is between 1:1 and 30:1. In relation to these embodiments, suitably the composition comprises 0.1-70 wt % aloe vera, suitably 1-60 wt % aloe vera, suitably 2-50 wt % aloe vera.

In an embodiment, the composition comprises 0.01-10 wt % curcumin compound; one or more glyceride(s) in a weight ratio of the curcumin compound to the glyceride(s) between 5:1000 and 100:1000; and aloe vera (e.g. aloe vera gel) in a weight ratio of the glyceride(s) to the aloe vera between 90:10 and 30:70. Such embodiments may also suitably comprise a gum (preferably xanthan gum) and glycerol, preferably in a weight ratio between 10:1 to 1:10 (more preferably between 5:1 and 2:1, most preferably about 3:1), wherein suitably the weight ratio of aloe vera to the combined weight of xanthan gum and glycerol is between 5:1 and 20:1. In relation to these embodiments, suitably the composition comprises 20-80 wt % aloe vera, suitably 30-70 wt % aloe vera, suitably 40-60 wt % aloe vera.

It will be readily appreciated that any of the aforesaid embodiments (including the features thereof, especially where defined relatively broadly) may be further defined and/or sub-defined as set forth herein, in particular (and where compatible in the given context) as set forth in paragraphs B1-B450 below.

### Absent/Low-Level Ingredients

Preferably, the composition is (substantially, preferably entirely) free of polymers. The composition is preferably (substantially, preferably entirely) free of polyvinyl alcohol (PVA). Preferably, the composition is (substantially, preferably entirely) free of polyalkylene glycol(s) (e.g. polyethylene glycol and/or polypropylene glycol). Preferably, the composition is (substantially, preferably entirely) free of polyalkylene glycol ester(s) (e.g. polyethylene glycol and/or polypropylene glycol ester(s)). Preferably, the composition is (substantially, preferably entirely) free of any compounds that comprise polyalkylene glycol ester moieties (e.g. polyethylene glycol and/or polypropylene glycol ester moieties). Preferably, the composition is (substantially, preferably entirely) free of polyethylene glycol. Preferably, the composition is (substantially, preferably entirely) free of polyethylene glycol ester(s). Preferably, the composition is (substantially, preferably entirely) free of any compounds that comprise polyethylene glycol ester moieties.

Preferably, the composition is (substantially, preferably entirely) free of surfactants. Preferably, the composition is (substantially, preferably entirely) free of poloxamer surfactants. Preferably, the composition is (substantially, preferably entirely) free of poloxamer 407 (e.g. Pluronic F127). Preferably, the composition is (substantially, preferably entirely) free of polysorbate surfactants. Preferably, the composition is (substantially, preferably entirely) free of polysorbate 20. Preferably, the composition is (substantially, preferably entirely) free of PEG-40 castor oil (e.g. Killiphor RH 40, Cremophor RH40) surfactants. Preferably, the composition is (substantially, preferably entirely) free of poloxamer and polysorbate surfactants. Preferably, the composition is (substantially, preferably entirely) free of poloxamer. polysorbate, and PEG-40 castor oil surfactants. Preferably, the composition comprises at most 3 wt % surfactant(s), suitably at most 3 wt % of any surfactants defined in any of B255-B262 (especially poloxamer. polysorbate, and PEG-40 castor oil surfactants).

Preferably, the composition comprises at most 1 wt % surfactant(s), suitably at most 1 wt % of any surfactants defined in any of B255-B262 (especially poloxamer. polysorbate, and PEG-40 castor oil surfactants). Preferably, the composition comprises at most 0.1 wt % surfactant(s), suitably at most 0.1 wt % of any surfactants defined in any of B255-B262 (especially poloxamer. polysorbate, and PEG-40 castor oil surfactants). Preferably, the composition comprises at most 0.01 wt % surfactant(s), suitably at most 0.01 wt % of any surfactants defined in any of B255-B262 (especially poloxamer. polysorbate, and PEG-40 castor oil surfactants). Preferably, the composition comprises at most 0.001 wt % surfactant(s), suitably at most 0.001 wt % of any surfactants defined in any of B255-B262 (especially poloxamer. polysorbate, and PEG-40 castor oil surfactants).

Suitably, the composition is (substantially, preferably entirely) free of lipids other than those stipulated.

Preferably, the composition is (substantially, preferably entirely) free of solvents (i.e. compounds that are liquids at SATP) other than water.

### Other Composition-Specific Facets

Suitably, at least 1 wt % of the composition is constituted by the stipulated ingredients (i.e. those listed as being present or optionally present within the composition). Suitably, at least 10 wt % of the composition is constituted by the stipulated ingredients. Suitably, at least 15 wt % of the composition is constituted by the stipulated ingredients. Suitably, at least 20 wt % of the composition is constituted by the stipulated ingredients. Suitably, at least 50 wt % of the composition is constituted by the stipulated ingredients. Suitably, at least 70 wt % of the composition is constituted by the stipulated ingredients. Suitably, at least 80 wt % of the composition is constituted by the stipulated ingredients. Suitably, at least 85 wt % of the composition is constituted by the stipulated ingredients. Suitably, at least 90 wt % of the composition is constituted by the stipulated ingredients. Suitably, at least 95 wt % of the composition is constituted by the stipulated ingredients. Suitably, at least 99 wt % of the composition is constituted by the stipulated ingredients. Suitably, the composition consists of the stipulated ingredients.

Suitably, at least 1 wt % of the composition is constituted by the stipulated curcumin compound and stipulated glyceride(s) (e.g. as per the glyceride(s) stipulated in B87-B144). Suitably, at least 10 wt % of the composition is constituted by the stipulated curcumin compound and stipulated glyceride(s). Suitably, at least 15 wt % of the composition is constituted by the stipulated curcumin compound and stipulated glyceride(s). Suitably, at least 20 wt % of the composition is constituted by the stipulated curcumin compound and stipulated glyceride(s). Suitably, at least 50 wt % of the composition is constituted by the stipulated curcumin compound and stipulated glyceride(s). Suitably, at least 70 wt % of the composition is constituted by the stipulated curcumin compound and stipulated glyceride(s). Suitably, Suitably, at least 80 wt % of the composition is constituted by the stipulated curcumin compound and stipulated glyceride(s). Suitably, at least 85 wt % of the composition is constituted by the stipulated curcumin compound and stipulated glyceride(s). Suitably, at least 90 wt % of the composition is constituted by the stipulated curcumin compound and stipulated glyceride(s). Suitably, at least 95 wt % of the composition is constituted by the stipulated curcumin compound and stipulated glyceride(s). Suitably, at least 99 wt % of the composition is constituted by the stipulated curcumin compound and stipulated glyceride(s).

Suitably, centrifugation (e.g. 3000 rpm for 5 min at RT) of the composition yields less than 10 wt % sedimentation (i.e. less than 10 wt % of the composition as a whole). Suitably, centrifugation (e.g. 3000 rpm for 5 min at RT) of the composition yields less than 5 wt % sedimentation (i.e. less than 5 wt % of the composition as a whole). Suitably, centrifugation (e.g. 3000 rpm for 5 min at RT) of the composition yields less than 2 wt % sedimentation (i.e. less than 2 wt % of the composition as a whole). Suitably, centrifugation (e.g. 3000 rpm for 5 min at RT) of the composition yields less than 1 wt % sedimentation (i.e. less than 1 wt % of the composition as a whole). Suitably, centrifugation (e.g. 3000 rpm for 5 min at RT) of the composition yields less than 0.1 wt % sedimentation (i.e. less than 0.1 wt % of the composition as a whole). Suitably, centrifugation (e.g. 3000 rpm for 5 min at RT) of the composition yields less than 0.01 wt % sedimentation (i.e. less than 0.01 wt % of the composition as a whole). Suitably, centrifugation (e.g. 3000 rpm for 5 min at RT) of the composition yields (substantially) no sedimentation.

Suitably, centrifugation (e.g. 3000 rpm for 5 min at RT) of the composition yields either no sedimentation or sedimentation containing less than 50 wt % of the curcumin compound present within the composition (i.e. this refers to 50 wt % of the total amount of curcumin compound, not 50 wt % of the composition as a whole). Suitably, centrifugation (e.g. 3000 rpm for 5 min at RT) of the composition yields either no sedimentation or sedimentation containing less than 20 wt % of the curcumin compound present within the composition. Suitably, centrifugation (e.g. 3000 rpm for 5 min at RT) of the composition yields either no sedimentation or sedimentation containing less than 10 wt % of the curcumin compound present within the composition. Suitably, centrifugation (e.g. 3000 rpm for 5 min at RT) of the composition yields either no sedimentation or sedimentation containing less than 5 wt % of the curcumin compound present within the composition. Suitably, centrifugation (e.g. 3000 rpm for 5 min at RT) of the composition yields either no sedimentation or sedimentation containing less than 1 wt % of the curcumin compound present within the composition. Suitably, centrifugation (e.g. 3000 rpm for 5 min at RT) of the composition yields either no sedimentation or sedimentation containing less than 0.1 wt % of the curcumin compound present within the composition. Suitably, centrifugation (e.g. 3000 rpm for 5 min at RT) of the composition yields either no sedimentation of any curcumin compound present within the composition.

### Method of Preparing Composition

The present invention provides a method of preparing a composition (suitably as defined herein), said method being suitably as defined herein. The method suitably involves mixing together all of the ingredients defined in respect of the composition, including such compositions as defined hereinbefore and hereinafter, including in numbered paragraphs A1-A131, B1-B450, and combinations thereof. As such, the method suitably comprises mixing a curcumin compound with the one or more glycerides. Suitably, the curcumin compound is mixed with the one or more glycerides at a temperature at which the one or more glycerides are in a melted phase (e.g. 30-90° C., suitably 35-70° C., most preferably 40-45° C.), suitably for sufficient time to ensure homogenisation of the resultant composition. The composition may be optionally cooled or allowed to cool, and optionally stored at room temperature (e.g. 15-25° C., or SATP) or under refrigeration, (e.g. 1-8° C.). Such a method is particularly apt for preparing an unhydrated/dehydrated composition (which preferably has at most 5 wt % water), though in principle water may be added to the mixture, suitably whilst the glyceride(s) are in the melted phase.

Where a moderately-hydrated composition is desired (e.g. 10-60 wt % water), suitably this is prepared by a method comprising:
i) performing the aforesaid method to provide an unhydrated/dehydrated composition;
ii) adding further curcumin compound to the unhydrated/dehydrated composition to furnish an enriched composition;
iii) adding water to the enriched composition; and
iv) mixing to form a hydrated-lipid-based mixture, suitably at a temperature at which the one or more glycerides are in a melted phase (e.g. 30-90° C., suitably 35-70° C., most preferably 40-45° C.), suitably for sufficient time to ensure homogenisation of the hydrated-lipid-based mixture, and optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.);
   wherein suitably step ii) is performed before step iii).

Where a very-hydrated composition (e.g. aqueous dispersion or aqueous emulsion) is desired (e.g. 85-99.9 wt % water), suitably this is prepared by a method comprising:
i) performing one of the aforesaid methods to provide either an unhydrated/dehydrated composition or a moderately hydrated composition;
ii) mixing the composition with the same weight or more of water (to provide a desired dilution), optionally in the presence of a surfactant (though suitably a surfactant as defined herein, suitably excluding certain surfactants), suitably at elevated temperatures (e.g. 30-90° C., suitably 35-70° C., most preferably 40-45° C.), optionally with ultra-sonication, to produce an emulsion or cubosome dispersion;
iii) optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.).

Any of the aforesaid methods may additionally comprise adding (or mixing in, preferably whilst the glycerides are in a melted phase) one or more additional ingredients (suitably as defined herein in relation to the composition).

The method may further comprise taking the product of the aforesaid method(s) (or a sample thereof) and subjecting it to centrifugation (e.g. 3000 rpm for 5 min at RT) to verify that one or more of the sedimentation requirements, defined herein, are met (e.g. as per B436-B450).

The present invention also provides a composition obtained by or directly obtained by the aforesaid method(s).

### Method of Preparing Other Products Using Composition

The present invention provides a product comprising a composition as defined herein. The product may suitably incorporate the composition.

The present invention also provides a method of preparing said product, the method comprising incorporating the composition within a product, suitably by mixing the composition with one or more additional ingredients (optionally as defined elsewhere herein) or one or more product ingredients (i.e. other ingredients used to make the product), or by incorporating the composition within an appropriate container or delivery device. For instance, in the case of a pharmaceutical product (e.g. a tablet), product ingredient(s) may include pharmaceutically acceptable excipient(s) or carrier(s). In the case the product is a spray, the composition may be mixed with one or more additional ingredients or product ingredients and incorporated within a spray device (e.g. having a reservoir, a spray nozzle, and a conduit connecting the contents of the reservoir with the spray nozzle).

The present invention also provides a product obtained by or directly obtained by the aforesaid method.

The product may suitably be a topical product (e.g. for use on a bodily surface, for example skin, body cavity, eye(s), hair). For example, the topical product may be a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical product that is a cream or ointment. In a particular embodiment, the product is a cream, an ointment, a foam, or a perfume (e.g. perfume, aftershave, cologne, or eau de toilette). In a particular embodiment, the product is a cream. In another embodiment, the product is an ointment.

In an alternative embodiment, the product may be an oral topical product, for example, a mouthwash, a rinse, an oral spray, a suspension, and/or a dental gel, suitably which is intended to be taken by mouth but are not intended to be ingested.

In an alternative embodiment, the product is an ingestible product (e.g. orally-administrable product). Such an ingestible product may be selected from a lozenge, a gel, a jelly, a pastille, a tablet, a capsule, a toffee, a nougat, a chewy candy, and/or a chewing gum. In a particular embodiment, the ingestible product is (or is otherwise incorporated within) a pastille, a gel, or a jelly. In another embodiment, the ingestible product comprises a gel component, gelling component, or gellable component. The gel component, gelling component, or gellable component is preferably animal- or plant-derived, most preferably plant-derived. In fact, most preferably, the product is free of any animal-derived components/ingredients. The gel component, gelling component, or gellable component may comprise or consist of a polysaccharide or derivative thereof (e.g. x-carrageenan). The gel component, gelling component, or gellable component may comprise or consists of a gelatin, agar, and/or agarose. The ingestible product may be a liquid, suitably a suspension, a dispersion, an emulsion, or a solution, most preferably a dispersion or emulsion of the aforesaid composition. The ingestible product may be a drink/beverage comprising the aforesaid composition. The drink/beverage may be an alcoholic or non-alcoholic drink/beverage.

In an embodiment, the product is a pharmaceutical product. Such a pharmaceutical product is suitably for use as a medicament (or may otherwise be used in the manufacture of a medicament). Such a pharmaceutical product may be an ayurvedic medicine. In an embodiment, the product composition is for (or is for use in) treating: neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukaemia, and colorectal cancer), anaphylaxis, arthritis, osteoarthritis, acne, and/or eczema.

The product may suitably be a nutraceutical product.

The product may suitably be a food supplement.

The product may suitably be a food additive.

The product may suitably be a foodstuff.

The product may be in the form of an emulsion, a lotion, a milk, a liquid, a solid, a cream, a gel, a mousse, an ointment, a paste, a serum, a stick, a spray, a tonic, an aerosol, a foam, and/or a pencil. The product may be an oil-in-water dispersion. The product may be a water-in-oil dispersion.

In an embodiment, the product is a personal care product. Said personal care product may be selected from the group consisting of skin care product, skin creams, shaving products (e.g. shaving creams, gels, foams), moisturizers, lotions, body washes, body oils, and hair care products (e.g. shampoos and/or hair conditioners). The personal care product may be a “leave-on-type” product (e.g. moisturizing lotions, serums, creams) or a “rinse-off-type” product (e.g. body washes, shampoos, hair conditioners, shower gels, skin cleansers, cleansing milks, shaving compositions). The personal care product may be in the form of an implement (e.g. a powdered or unpowdered cosmetic applicator), a reusable or disposable wipe, a tissue, a towel, a diaper, a razor or other shaving device, a personal cleansing implement (e.g. a mesh shower sponge), a conventional sponge, a wash cloth, a swab, and/or a pen.

In an embodiment, the product is a cosmetic product. The cosmetic product may be for controlling and/or reducing the formation of wrinkles and lines in the skin. The cosmetic product may be for providing glowing skin.

In an embodiment, the product is a wound care product. The wound care (or wound healing) product may be a wound care hydrogel composition, and therefor suitably comprises a hydrogel or hydrogel-forming component or compound—this may be a polysaccharide-based hydrogel or hydrogel-forming component or compound. Such a wound care product may be selected from the group consisting of a wound dressing, a sutures, a staple, a gauze, a bandage, a plaster, a burn dressing, an artificial skin, a liposome or micelle formulation, a microcapsule, an aqueous vehicle for soaking gauze dressings. In an embodiment, the wound care product may be for (or for use in) healing wounds, for example, healing cuts and scrapes, burns, ulcers, bed sores, fissures, haemorrhoids, and/or post-surgical wounds. The same properties that render compositions of the invention excellent for topical applications also apply to wound care products.

The product may be a textile—e.g. a textile (e.g. a clothing item) comprising the aforesaid composition.

Whilst the composition of the invention may be a foodstuff or a food additive, preferably the composition of the invention is not a foodstuff or a food additive. Likewise, preferably any product formed from the composition of the invention is not a foodstuff or a food additive. Preferably the product is not a food supplement, food additive, or a foodstuff.

### Uses of Composition and Products Formed Therefrom

Compositions and products of the invention may be used in a variety of ways, as defined herein.

By way of example, the present invention provides a use of a composition (as defined herein) or product (as defined herein) for topical application to bodily surfaces (e.g. skin, eye(s), hair, body cavity), for topical application to the mouth, for oral administration/ingestion. Sub-definitions of such uses will be readily apparent from recitations of these general categories throughout this specification—for instance, paragraphs B2-B43 elucidate such uses further.

The present invention also provides a use of a composition (as defined herein) or product (as defined herein) for pharmaceutical treatments, for nutraceutical treatments, for cosmetic treatments/applications, for dental treatments, for eye treatments, in foods, for personal care (e.g. skin care, hair care), for wound care. Again, sub-definitions of all such uses will be readily apparent from recitations of these general categories throughout this specification—for instance, paragraphs B2-B43 elucidate such uses further.

### Medical Uses

The present invention provides a composition (e.g. a pharmaceutical composition), suitably as defined herein, or a product (e.g. pharmaceutical product), suitably as defined herein, for use as a medicament (or for use in treating a disease, disorder, or medical condition, suitably a disease, disorder, or medical condition as defined herein).

The present invention provides a use of a composition (e.g. a pharmaceutical composition), suitably as defined herein, or a product (e.g. pharmaceutical product), suitably as defined herein, in the manufacture of a medicament (or in the manufacture of a medicament for treating a disease, disorder, or medical condition, suitably a disease, disorder, or medical condition as defined herein).

The present invention provides a method of treating a disease, disorder, or medical condition (suitably a disease, disorder, or medical condition as defined herein) in a subject (suitably in a subject identified as in need of treatment), the method comprising administering to the subject a therapeutically effective amount of a composition (e.g. a pharmaceutical composition), suitably as defined herein, or a product (e.g. pharmaceutical product), suitably as defined herein.

Suitably, the disease, disorder, or medical condition comprises neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukaemia, and colorectal cancer), anaphylaxis, arthritis, osteoarthritis, acne, and/or eczema.

Suitably, administering the composition or the product to the subject comprises topical administration or oral administration, most preferably topical administration.

Compositions of the invention, and/or products incorporating the same, maybe used as an immunity booster—i.e. to enhance the performance of the immune system (suitably of a human or animal subject).

### Specific Embodiments

The examples and data presented herein suggest that certain technical effects may arise from certain features or feature combinations, in light of which various specific embodiments A1-A131 are discussed below. A person skilled in the art will readily appreciate embodiments elucidated by the dependencies set forth below, including via direct and indirect dependencies. The embodiments disclosed in this section may be combined with any embodiments and features disclosed in any other section herein.

### A) Specific Embodiments

A1. A composition comprising a curcumin compound and one or more glycerides (which, together with any extra, are ingredients of the composition), wherein at least one (preferably each) of the glyceride(s) comprises at least one unsaturated moiety (preferably at least one alkene moiety, most preferably at least one cis-alkene moiety), suitably wherein the composition comprises at most 10 wt % water (preferably at most 5 wt % water, most preferably at most 2 wt % water).
A2. A composition comprising a curcumin compound and one or more monoglycerides, wherein at least one of the one or more monoglyceride(s) comprises at least one cis-alkene moiety. In this context, references herein to glyceride(s) are references to the monoglyceride(s).
A3. A composition comprising a curcumin compound and one or more monoglycerides, wherein at least one of the one or more monoglyceride(s) comprises at least one cis-alkene moiety; on the proviso that the composition is not a food supplement, food additive, or a foodstuff. In this context, references herein to glyceride(s) are references to the monoglyceride(s).
A4. A composition comprising a curcumin compound, one or more glycerides, and water (which, together with any extra, are ingredients of the composition), wherein at least one (preferably each) of the glyceride(s) comprises at least one unsaturated moiety (preferably at least one alkene moiety, most preferably at least one cis-alkene moiety).
A5. A composition comprising one or more curcumin compounds (preferably wherein one of the curcumin compounds constitutes a “main” curcumin compound, typically referred herein to “a curcumin compound” or “the curcumin compound”, most preferably the “main” curcumin compound is curcumin or a pharmaceutically acceptable salt thereof); and one or more glycerides; suitably wherein at least one (preferably each) of the glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.
A6. A composition comprising:
   a curcumin compound (or one or more curcumin compounds, suitably wherein one curcumin compound is a “main” curcumin compound); and
   one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides;
   wherein the curcumin compound is provided by a turmeric extract (and/or curcuminoid mixture) that is included within the composition;
   wherein optionally the composition further comprises water, optionally in combination with a surfactant where the composition is a water-in-oil or oil-in-water dispersion or emulsion.
A7. A composition comprising:
   a curcumin compound (or one or more curcumin compounds, suitably wherein one curcumin compound is a “main” curcumin compound);
   one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides; and
   one or more oils, preferably one or more oils selected from the group consisting of moringa oil, coconut oil, sunflower oil, avocado oil, soya oil (or soybean oil), corn oil, or any combination thereof;
   wherein optionally the composition further comprises water, optionally in combination with a surfactant where the composition is a water-in-oil or oil-in-water dispersion or emulsion.
A8. A composition comprising:
   a curcumin compound (or one or more curcumin compounds, suitably wherein one curcumin compound is a “main” curcumin compound);
   one or more lipids, which are preferably one or more glycerides, suitably wherein at least one (preferably each) of glyceride(s) comprises at least one unsaturated moiety (preferably at least one alkene moiety, most preferably at least one cis-alkene moiety), suitably wherein the one or more glyceride(s) are one or more mono- and/or di-glycerides, though most preferably are one or more mono-glycerides; and
   one or more oils, preferably one or more oils selected from the group consisting of moringa oil, coconut oil, sunflower oil, avocado oil, soya oil (or soybean oil), corn oil, or any combination thereof;
   wherein the curcumin compound is provided by a turmeric extract (and/or curcuminoid mixture) that is included within the composition;
   wherein optionally the composition further comprises water, optionally in combination with a surfactant where the composition is a water-in-oil or oil-in-water dispersion or emulsion.
A9. A method of preparing the composition of A1, the method comprising mixing the curcumin compound with the one or more glycerides (i.e. mixing the ingredients of the composition, which may include additional ingredients, such as those stipulated hereinafter in relation to the composition) to form a lipid-based mixture, suitably at a temperature at which the one or more glycerides are in a melted phase (e.g. 30-90° C., suitably 35-70° C., most preferably 40-45° C.), suitably for sufficient time to ensure homogenisation of the lipid-based mixture, and optionally cooling the lipid-based mixture (or allowing the lipid-based mixture to cool) and optionally thereafter storing (e.g. at room temperature, e.g. 15-25° C. or under refrigeration, e.g. 1-8° C.).
A10. A method of preparing the composition of A4, the method comprising:
   i) performing the method of A9 or otherwise providing the composition of A1;
   ii) adding further curcumin compound to the composition;
   iii) adding water (now a further ingredient of the composition) to the composition;
   iv) mixing to form a hydrated-lipid-based mixture, suitably at a temperature at which the one or more glycerides are in a melted phase (e.g. 30-90° C., suitably 35-70° C., most preferably 40-45° C.), suitably for sufficient time to ensure homogenisation of the hydrated-lipid-based mixture, and optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.);
   wherein suitably step ii) is performed before step iii).
A11. A method of preparing the composition of A4, the method comprising:
   i) performing the method of A9 or A10 or otherwise providing the composition of A1 or A4;
   ii) mixing the composition with the same weight or more of water (to provide a desired dilution), optionally in the presence of a surfactant (though suitably a surfactant as defined herein, suitably excluding certain surfactants), suitably at elevated temperatures (e.g. 30-90° C., suitably 35-70° C., most preferably 40-45° C.), optionally with ultra-sonication, to produce an emulsion or cubosome dispersion;
   iii) optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.).
A12. The method of any of A9-A11, wherein the method comprises adding (or mixing in) one or more additional ingredients.
A13. A product comprising the composition of any of A1-A8 or a composition obtained by (or directly obtained by) the method of A9-A11.
A14. A method of preparing the product of A13, the method comprising incorporating the composition of A1-A8 or a composition obtained by (or directly obtained by) the method of A9-A11 within a product, suitably by mixing the composition with one or more additional ingredients (optionally as defined elsewhere herein) or one or more product ingredients (i.e. other ingredients used to make the product), or by incorporating the composition within an appropriate container or delivery device.
A15. A composition (e.g. a pharmaceutical composition) of A1-A8 or a composition obtained by (or directly obtained by) the method of A9-A11, or a product (e.g. pharmaceutical product) of A13 or product obtained (or directly obtained by) the method of A14, for use as a medicament (or for use in treating a disease, disorder, or medical condition, suitably a disease, disorder, or medical condition as defined herein).
A16. Use of a composition (e.g. a pharmaceutical composition) of A1-A8 or a composition obtained by (or directly obtained by) the method of A9-A11, or a product (e.g. pharmaceutical product) of A13 or product obtained (or directly obtained by) the method of A14, in the manufacture of a medicament (or in the manufacture of a medicament for treating a disease, disorder, or medical condition, suitably a disease, disorder, or medical condition as defined herein).
A17. A method of treating a disease, disorder, or medical condition (suitably a disease, disorder, or medical condition as defined herein) in a subject (suitably in a subject identified as in need of treatment), the method comprising administering to the subject a therapeutically effective amount of the composition (e.g. a pharmaceutical composition) of A1-A8 or a composition obtained by (or directly obtained by) the method of A9-A11, or a product (e.g. pharmaceutical product) of A13 or product obtained (or directly obtained by) the method of A14.
A18. The composition, method, product, or use of any of A1-A17, wherein the one or more glycerides are one or more monoglycerides bearing a C₈-C₂₀ fatty acid moiety having at least one cis-alkene.
A19. The composition, method, product, or use of A18, wherein the composition (i.e. ingredients of the composition) comprises a curcumin compound, one or more monoglycerides bearing a C₈-C₂₀ fatty acid moiety having at least one cis-alkene, and bile salts (and/or bile acid(s)).
A20. The composition, method, product, or use of A18, wherein the composition (i.e. ingredients of the composition) comprises curcumin, glyceryl monooleate, glyceryl monolinoleate, and bile salts (and/or bile acid(s)).
A21. The composition, method, product, or use of any of A18-A19, wherein the composition (i.e. ingredients of the composition) comprises the curcumin compound, a first monoglyceride, and a second monoglyceride, wherein both the first monoglyceride and second monoglyceride are compounds comprising at least one alkene moiety (most preferably at least one cis-alkene moiety), wherein suitably the first monoglyceride and second monoglyceride are present in a weight ratio between 20:80 and 40:60.
A22. The composition, method, product, or use of A21, wherein the composition comprises a curcumin compound (preferably curcumin itself), glyceryl monooleate, and glyceryl monolinoleate, wherein suitably glyceryl monooleate and glyceryl monolinoleate are present in a weight ratio between 20:80 and 40:60.
A23. The composition, method, product, or use of A21, wherein the composition comprises a curcumin compound (preferably curcumin itself), glyceryl monooleate, and glyceryl monolinoleate, wherein suitably glyceryl monooleate and glyceryl monolinoleate are present in a weight ratio between 20:80 and 40:60.
A24. The composition, method, product, or use of A21, wherein the composition comprises a curcumin compound (preferably curcumin itself), glyceryl monooleate, glyceryl monolinoleate, and water, wherein water is at a concentration within the composition of 10-60 wt %, wherein suitably glyceryl monooleate and glyceryl monolinoleate are present in a weight ratio between 20:80 and 40:60.
A25. The composition, method, product, or use of A21, wherein the composition comprises a curcumin compound (preferably curcumin itself), glyceryl monooleate, glyceryl monolinoleate, water, and bile salts (and/or bile acid(s)), wherein water is at a concentration within the composition of 10-60 wt %, wherein suitably glyceryl monooleate and glyceryl monolinoleate are present in a weight ratio between 20:80 and 40:60.
A26. The composition, method, product, or use of A1-A25, wherein the composition comprises a curcumin compound (preferably curcumin itself), glyceryl monooleate, glyceryl monolinoleate, and bile salts (and/or bile acid(s)), wherein suitably glyceryl monooleate and glyceryl monolinoleate are present in a weight ratio between 20:80 and 40:60.
A27. The composition, method, product, or use of any of A1-A17, wherein the composition comprises 0.5-800 mg/g curcumin compound, and one or more monoglycerides selected from glyceryl monooleate, glyceryl monolinoleate, or any combination thereof. NB. The 0.5-800 mg/g refers to curcumin only.
A28. The composition, method, product, or use of any of A1-A17, wherein the composition comprises 0.1-50 wt % curcumin compound, and one or more monoglycerides bearing a C₈-C₂₀ fatty acid moiety having at least one cis-alkene, wherein the weight ratio of the curcumin compound to the one or more monoglyceride(s) is between 0.5:1000 and 800:1000 (i.e. 5:10000-8:10). NB. The 0.1-50 wt % applies to curcumin only.
A29. The composition, method, product, or use of any of A1-A17, wherein the composition comprises curcumin and glyceryl monooleate, wherein: the composition has at most 5 wt % water.
A30. The composition, method, product, or use of any of A1-A17, wherein the composition comprises 1-10 mg/g curcumin, and glyceryl monooleate, wherein: the composition has at most 5 wt % water; and at least 20 wt % of the composition is constituted by the curcumin, glyceryl monooleate, and water (to the extent that any water is present). NB. The 1-10 mg/g refers to the concentration of curcumin only within the composition, and does not include any subsequently-listed ingredients without quantity qualifications. Subsequently listed ingredients with unstated concentrations are not limited by a concentration save to the extent indicated by other features of the embodiment. This pattern of stipulating a concentration for curcumin only, and not for other subsequently-listed ingredients, applies throughout unless otherwise stated.
A31. The composition, method, product, or use of any of A1-A17, wherein the composition comprises curcumin and glyceryl monooleate, wherein: the weight ratio of curcumin to glyceryl monooleate is between 2:1000 and 8:1000; the composition has at most 5 wt % water; and at least 20 wt % of the composition is constituted by the curcumin, glyceryl monooleate, and water (to the extent that any water is present).
A32. The composition, method, product, or use of any of A1-A17, wherein the composition comprises 2-8 mg/g curcumin, and glyceryl monooleate, wherein: the composition has at most 2 wt % water; and at least 80 wt % of the composition is constituted by the curcumin, glyceryl monooleate, and water (to the extent that any water is present).
A33. The composition, method, product, or use of any of A1-A17, wherein the composition comprises curcumin and glyceryl monooleate, wherein: the weight ratio of curcumin to glyceryl monooleate is between 2:1000 and 8:1000; the composition has at most 2 wt % water; and at least 80 wt % of the composition is constituted by the curcumin, glyceryl monooleate, and water (to the extent that any water is present).
A34. The composition, method, product, or use of any of A1-A17, wherein the composition comprises 3-7 mg/g curcumin, and glyceryl monooleate, wherein: the composition has at most 2 wt % water; at least 80 wt % of the composition is constituted by the curcumin, glyceryl monooleate, and water (to the extent that any water is present); the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A35. The composition, method, product, or use of any of A1-A17, wherein the composition comprises curcumin and glyceryl monooleate, wherein: the weight ratio of curcumin to glyceryl monooleate is between 4:1000 and 6:1000; the composition has at most 2 wt % water; at least 80 wt % of the composition is constituted by the curcumin, glyceryl monooleate, and water (to the extent that any water is present); the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A36. The composition, method, product, or use of any of A1-A17, wherein the composition comprises curcumin and glyceryl monooleate, wherein: the composition comprises 10-60 wt % water.
A37. The composition, method, product, or use of any of A1-A17, wherein the composition comprises 5-30 mg/g curcumin, and glyceryl monooleate, wherein: the composition comprises 10-60 wt % water; and at least 20 wt % of the composition is constituted by the curcumin, glyceryl monooleate, and water.
A38. The composition, method, product, or use of any of A1-A17, wherein the composition comprises curcumin and glyceryl monooleate, wherein: the weight ratio of curcumin to glyceryl monooleate is between 5:1000 and 30:1000; the composition comprises 10-60 wt % water; and at least 20 wt % of the composition is constituted by the curcumin, glyceryl monooleate, and water.
A39. The composition, method, product, or use of any of A1-A17, wherein the composition comprises 10-20 mg/g curcumin, and glyceryl monooleate, wherein: the composition comprises 20-45 wt % water; and at least 80 wt % of the composition is constituted by the curcumin, glyceryl monooleate, and water.
A40. The composition, method, product, or use of any of A1-A17, wherein the composition comprises curcumin and glyceryl monooleate, wherein: the weight ratio of curcumin to glyceryl monooleate is between 11:1000 and 19:1000; the composition comprises 20-45 wt % water; and at least 80 wt % of the composition is constituted by the curcumin, glyceryl monooleate, and water.
A41. The composition, method, product, or use of any of A1-A17, wherein the composition comprises 10-20 mg/g curcumin, and glyceryl monooleate, wherein: the composition comprises 20-45 wt % water; at least 80 wt % of the composition is constituted by the curcumin, glyceryl monooleate, and water; the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A42. The composition, method, product, or use of any of A1-A17, wherein the composition comprises curcumin and glyceryl monooleate, wherein: the weight ratio of curcumin to glyceryl monooleate is between 11:1000 and 19:1000; the composition comprises 20-45 wt % water; at least 80 wt % of the composition is constituted by the curcumin, glyceryl monooleate, and water; the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A43. The composition, method, product, or use of any of A1-A17, wherein the composition comprises curcumin, glyceryl monooleate, and glyceryl monolinoleate, wherein: the composition has at most 5 wt % water.
A44. The composition, method, product, or use of any of A1-A17, wherein the composition comprises 2-13 mg/g curcumin, glyceryl monooleate, and glyceryl monolinoleate, wherein: the composition has at most 5 wt % water; and at least 20 wt % of the composition is constituted by the curcumin, glyceryl monooleate, glyceryl monolinoleate, and water (to the extent that any water is present).
A45. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 5:1000 and 10:1000; the composition has at most 5 wt % water; and at least 20 wt % of the composition is constituted by the curcumin, glyceryl monooleate, glyceryl monolinoleate, and water (to the extent that any water is present).
A46. The composition, method, product, or use of any of A1-A17, wherein the composition comprises 5-10 mg/g curcumin, glyceryl monooleate, and glyceryl monolinoleate, wherein: the composition has at most 2 wt % water; and at least 80 wt % of the composition is constituted by the curcumin, glyceryl monooleate, glyceryl monolinoleate, and water (to the extent that any water is present).
A47. The composition, method, product, or use of any of A1-A17, wherein the composition comprises curcumin, glyceryl monooleate, and glyceryl monolinoleate wherein: the weight ratio of curcumin to glyceryl monooleate and glyceryl monolinoleate combined is between 6.5:1000 and 8.5:1000; the composition has at most 2 wt % water; and at least 80 wt % of the composition is constituted by the curcumin, glyceryl monooleate, glyceryl monolinoleate, and water (to the extent that any water is present).
A48. The composition, method, product, or use of any of A1-A17, wherein the composition comprises 5-10 mg/g curcumin, glyceryl monooleate, and glyceryl monolinoleate, wherein: the composition has at most 2 wt % water; at least 80 wt % of the composition is constituted by the curcumin, glyceryl monooleate, glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A49. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 6.5:1000 and 8.5:1000; the composition has at most 2 wt % water; at least 80 wt % of the composition is constituted by the curcumin, glyceryl monooleate, glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A50. The composition, method, product, or use of any of A1-A17, wherein the composition comprises curcumin, glyceryl monooleate, and glyceryl monolinoleate, wherein: the composition comprises 10-60 wt % water.
A51. The composition, method, product, or use of any of A1-A17, wherein the composition comprises 10-40 mg/g curcumin, glyceryl monooleate, and glyceryl monolinoleate, wherein: the composition comprises 10-60 wt % water; and at least 20 wt % of the composition is constituted by the curcumin, glyceryl monooleate, glyceryl monolinoleate, and water.
A52. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 10:1000 and 40:1000; the composition comprises 10-60 wt % water; and at least 20 wt % of the composition is constituted by the curcumin, glyceryl monooleate, glyceryl monolinoleate, and water.
A53. The composition, method, product, or use of any of A1-A17, wherein the composition comprises 15-35 mg/g curcumin, glyceryl monooleate, and glyceryl monolinoleate wherein: the composition comprises 20-45 wt % water; and at least 80 wt % of the composition is constituted by the curcumin, glyceryl monooleate, glyceryl monolinoleate, and water.
A54. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 20:1000 and 30:1000; the composition comprises 20-45 wt % water; and at least 80 wt % of the composition is constituted by the curcumin, glyceryl monooleate, glyceryl monolinoleate, and water.
A55. The composition, method, product, or use of any of A1-A17, wherein the composition comprises 15-35 mg/g curcumin, glyceryl monooleate, and glyceryl monolinoleate, wherein: the composition comprises glyceryl monooleate and glyceryl monolinoleate in a weight ratio between 25:75 and 35:65; the composition comprises 20-45 wt % water; at least 80 wt % of the composition is constituted by the curcumin, glyceryl monooleate, glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A56. The composition, method, product, or use of any of A1-A17, wherein the composition comprises curcumin, glyceryl monooleate, and glyceryl monolinoleate wherein: the composition comprises glyceryl monooleate and glyceryl monolinoleate in a weight ratio between 25:75 and 35:65; the weight ratio of curcumin to glyceryl monooleate and glyceryl monolinoleate combined (i.e. relative to total weight of glyceryl monooleate and glyceryl monolinoleate combined) is between 20:1000 and 30:1000; the composition comprises 20-45 wt % water; at least 80 wt % of the composition is constituted by the curcumin, glyceryl monooleate, glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A57. The composition, method, product, or use of any of A1-A17, wherein the composition comprises 40-150 mg/g curcumin, glyceryl monooleate, and glyceryl monolinoleate, wherein: the composition has at most 5 wt % water; and at least 20 wt % of the composition is constituted by the curcumin, glyceryl monooleate, glyceryl monolinoleate, and water (to the extent that any water is present).
A58. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 40:1000 and 150:1000; the composition has at most 5 wt % water; and at least 20 wt % of the composition is constituted by the curcumin, glyceryl monooleate, glyceryl monolinoleate, and water (to the extent that any water is present).
A59. The composition, method, product, or use of any of A1-A17, wherein the composition comprises 40-150 mg/g curcumin, glyceryl monooleate, and glyceryl monolinoleate, wherein: the composition has at most 2 wt % water; and at least 80 wt % of the composition is constituted by the curcumin, glyceryl monooleate, glyceryl monolinoleate, and water (to the extent that any water is present).
A60. The composition, method, product, or use of any of A1-A17, wherein the composition comprises curcumin, glyceryl monooleate, and glyceryl monolinoleate wherein: the weight ratio of curcumin to glyceryl monooleate and glyceryl monolinoleate combined is between 60:1000 and 100:1000; the composition has at most 2 wt % water; and at least 80 wt % of the composition is constituted by the curcumin, glyceryl monooleate, glyceryl monolinoleate, and water (to the extent that any water is present).
A61. The composition, method, product, or use of any of A1-A17, wherein the composition comprises 60-100 mg/g curcumin, glyceryl monooleate, and glyceryl monolinoleate, wherein: the composition has at most 2 wt % water; at least 80 wt % of the composition is constituted by the curcumin, glyceryl monooleate, glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A62. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 65:1000 and 95:1000; the composition has at most 2 wt % water; at least 80 wt % of the composition is constituted by the curcumin, glyceryl monooleate, glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A63. The composition, method, product, or use of any of A1-A17, wherein the composition comprises 100-700 mg/g curcumin, glyceryl monooleate, and glyceryl monolinoleate, wherein: the composition comprises 10-60 wt % water; and at least 20 wt % of the composition is constituted by the curcumin, glyceryl monooleate, glyceryl monolinoleate, and water.
A64. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 100:1000 and 700:1000; the composition comprises 10-60 wt % water; and at least 20 wt % of the composition is constituted by the curcumin, glyceryl monooleate, glyceryl monolinoleate, and water.
A65. The composition, method, product, or use of any of A1-A17, wherein the composition comprises 200-600 mg/g curcumin, glyceryl monooleate, and glyceryl monolinoleate wherein: the composition comprises 20-45 wt % water; and at least 80 wt % of the composition is constituted by the curcumin, glyceryl monooleate, glyceryl monolinoleate, and water.
A66. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 300:1000 and 500:1000; the composition comprises 20-45 wt % water; and at least 80 wt % of the composition is constituted by the curcumin, glyceryl monooleate, glyceryl monolinoleate, and water.
A67. The composition, method, product, or use of any of A1-A17, wherein the composition comprises 300-500 mg/g curcumin, glyceryl monooleate, and glyceryl monolinoleate, wherein: the composition comprises glyceryl monooleate and glyceryl monolinoleate in a weight ratio between 25:75 and 35:65; the composition comprises 20-45 wt % water; at least 80 wt % of the composition is constituted by the curcumin, glyceryl monooleate, glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A68. The composition, method, product, or use of any of A1-A17, wherein the composition comprises curcumin, glyceryl monooleate, and glyceryl monolinoleate wherein: the composition comprises glyceryl monooleate and glyceryl monolinoleate in a weight ratio between 25:75 and 35:65; the weight ratio of curcumin to glyceryl monooleate and glyceryl monolinoleate combined (i.e. relative to total weight of glyceryl monooleate and glyceryl monolinoleate combined) is between 350:1000 and 450:1000; the composition comprises 20-45 wt % water; at least 80 wt % of the composition is constituted by the curcumin, glyceryl monooleate, glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A69. The composition, method, product, or use of any of A1-A17, wherein the composition comprises curcumin, glyceryl monooleate, and glyceryl monolinoleate, wherein: the composition comprises 40-85 wt % water; and at least 15 wt % of the composition is constituted by curcumin, glyceryl monooleate, and glyceryl monolinoleate.
A70. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 5:1000 and 30:1000; the composition comprises 40-85 wt % water; at least 15 wt % of the composition is constituted by curcumin, glyceryl monooleate, and glyceryl monolinoleate; the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A71. The composition, method, product, or use of any of A1-A17, wherein the composition comprises curcumin, glyceryl monooleate, and glyceryl monolinoleate wherein: the weight ratio of curcumin to glyceryl monooleate and glyceryl monolinoleate combined is between 5:1000 and 10:1000; the composition comprises 40-85 wt % water; at least 15 wt % of the composition is constituted by curcumin, glyceryl monooleate, and glyceryl monolinoleate; the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A72. The composition, method, product, or use of any of A1-A17, wherein the composition comprises curcumin, glyceryl monooleate, and glyceryl monolinoleate, wherein: the weight ratio of curcumin to glyceryl monooleate and glyceryl monolinoleate combined is between 6:1000 and 9:1000; the composition comprises 40-85 wt % water; at least 15 wt % of the composition is constituted by curcumin, glyceryl monooleate, and glyceryl monolinoleate; the composition is free of surfactants (or is free of free of poloxamer. polysorbate, and PEG-40 castor oil surfactants) or comprises at most 1 wt % surfactant(s) (or at most 1 wt % free of poloxamer. polysorbate, and PEG-40 castor oil surfactants); the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the 50 composition.
A73. The composition, method, product, or use of any of A1-A17, wherein the composition comprises curcumin, glyceryl monooleate, and glyceryl monolinoleate, wherein: the composition comprises at least 85 wt % water.
A74. The composition, method, product, or use of any of A1-A17, wherein the composition comprises curcumin, glyceryl monooleate, and glyceryl monolinoleate, wherein: the curcumin compound is present within the composition at a concentration greater than equal to 0.1 mg/g; the weight ratio of curcumin to glyceryl monooleate and glyceryl monolinoleate combined is between 40:1000 and 150:1000; the composition comprises at least 85 wt % water; and the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A75. The composition, method, product, or use of any of A1-A17, wherein the composition comprises curcumin, glyceryl monooleate, and glyceryl monolinoleate, wherein: the curcumin compound is present within the composition at a concentration greater than equal to 0.1 mg/g; the weight ratio of curcumin to glyceryl monooleate and glyceryl monolinoleate combined is between 60:1000 and 100:1000; the composition comprises at least 85 wt % water; and the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A76. The composition, method, product, or use of any of A1-A17, wherein the composition comprises curcumin, glyceryl monooleate, and glyceryl monolinoleate wherein: wherein the curcumin compound is present within the composition at a concentration greater than equal to 0.1 mg/g; the composition comprises glyceryl monooleate and glyceryl monolinoleate in a weight ratio between 25:75 and 35:65; the weight ratio of curcumin to glyceryl monooleate and glyceryl monolinoleate combined (i.e. relative to total weight of glyceryl monooleate and glyceryl monolinoleate combined) is between 65:1000 and 95:1000; the composition comprises at least 85 wt % water; the composition is free of surfactants (or is free of free of poloxamer. polysorbate, and PEG-40 castor oil surfactants) or comprises at most 1 wt % surfactant(s) (or at most 1 wt % free of poloxamer. polysorbate, and PEG-40 castor oil surfactants); the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. glyceryl monooleate, glyceryl monolinoleate, and any unstipulated glyceride(s)) constitute at least 95 wt % of the total amount of lipids present within the composition.
A77. The composition, method, product, or use of any of A1-A17, wherein the composition comprises 0.1-1 wt % curcumin compound and 95-99.9 wt % of the one or more glyceride(s).
A78. The composition, method, product, or use of any of A1-A17, wherein the composition consists of or consists essentially of 0.1-1 wt % curcumin compound and 95-99.9 wt % of the one or more glyceride(s).
A79. The composition, method, product, or use of any of A1-A17, wherein the composition comprises 0.1-1 wt % curcumin and 95-99.9 wt % of the one or more glyceride(s).
A80. The composition, method, product, or use of any of A1-A17, wherein the composition consists of or consists essentially of 0.1-1 wt % curcumin and 95-99.9 wt % of the one or more glyceride(s).
A81. The composition, method, product, or use of any of A1-A17, wherein the composition comprises 0.1-1 wt % curcumin and 95-99.9 wt % glyceryl monooleate.
A82. The composition, method, product, or use of any of A1-A17, wherein the composition consists of or consists essentially of 0.1-1 wt % curcumin and 99-99.9 wt % glyceryl monooleate.
A83. The composition, method, product, or use of any of A1-A17, wherein the composition comprises 0.5-1.5 wt % curcumin compound and 95-99.5 wt % of the one or more (preferably at least two, preferably two) glyceride(s).
A84. The composition, method, product, or use of any of A1-A17, wherein the composition consists of or consists essentially of 0.5-1.5 wt % curcumin compound and 95-99.5 wt % of the one or more (preferably at least two, preferably two) glyceride(s).
A85. The composition, method, product, or use of any of A1-A17, wherein the composition comprises 0.5-1.5 wt % curcumin and 95-99.5 wt % of the one or more (preferably at least two, preferably two) glyceride(s).
A86. The composition, method, product, or use of any of A1-A17, wherein the composition consists of or consists essentially of 0.5-1.5 wt % curcumin and 95-99.5 wt % of the one or more (preferably at least two, preferably two) glyceride(s).
A87. The composition, method, product, or use of any of A1-A17, wherein the composition comprises 0.5-1.5 wt % curcumin and 95-99.5 wt % mixture of glyceryl monooleate and glyceryl monolinoleate in a respective weight ratio between 25:75 and 35:65.
A88. The composition, method, product, or use of any of A1-A17, wherein the composition consists of or consists essentially of 0.5-1.5 wt % curcumin and 98.5-99.5 wt % mixture of glyceryl monooleate and glyceryl monolinoleate in a respective weight ratio between 25:75 and 35:65.
A89. The composition, method, product, or use of any of A1-A17, wherein the composition comprises 1-10 wt % (preferably 5-10 wt %) curcumin compound and 85-99 wt % (preferably 90-95 wt %) of the one or more (preferably at least two, preferably two) glyceride(s).
A90. The composition, method, product, or use of any of A1-A17, wherein the composition consists of or consists essentially of 1-10 wt % (preferably 5-10 wt %) curcumin compound and 85-99 wt % (preferably 90-95 wt %) of the one or more (preferably at least two, preferably two) glyceride(s).
A91. The composition, method, product, or use of any of A1-A17, wherein the composition comprises 1-10 wt % (preferably 5-10 wt %) curcumin and 85-99 wt % (preferably 90-95 wt %) of the one or more (preferably at least two, preferably two) glyceride(s).
A92. The composition, method, product, or use of any of A1-A17, wherein the composition consists of or consists essentially of 1-10 wt % (preferably 5-10 wt %) curcumin and 85-99 wt % (preferably 90-95 wt %) of the one or more (preferably at least two, preferably two) glyceride(s).
A93. The composition, method, product, or use of any of A1-A17, wherein the composition comprises 1-10 wt % (preferably 5-10 wt %) curcumin and 85-99 wt % (preferably 90-95 wt %) mixture of glyceryl monooleate and glyceryl monolinoleate in a respective weight ratio between 25:75 and 35:65.
A94. The composition, method, product, or use of any of A1-A17, wherein the composition consists of or consists essentially of 1-10 wt % (preferably 5-10 wt %) curcumin and 90-99 wt % (preferably 90-95 wt %) mixture of glyceryl monooleate and glyceryl monolinoleate in a respective weight ratio between 25:75 and 35:65.
A95. The composition, method, product, or use of any of A1-A17, wherein the composition comprises 0.1-3 wt % (preferably 0.5-1.5 wt %) curcumin compound, 35-85 wt % (preferably 55-65 wt %) of the one or more glyceride(s), and 15-65 wt % (preferably 35-45 wt %) water.
A96. The composition, method, product, or use of any of A1-A17, wherein the composition consists of or consists essentially of 0.1-3 wt % (preferably 0.5-1.5 wt %) curcumin compound, 35-85 wt % (preferably 55-65 wt %) of the one or more glyceride(s), and 15-65 wt % (preferably 35-45 wt %) water.
A97. The composition, method, product, or use of any of A1-A17, wherein the composition comprises 0.1-3 wt % (preferably 0.5-1.5 wt %) curcumin, 35-85 wt % (preferably 55-65 wt %) of the one or more glyceride(s), and 15-65 wt % (preferably 35-45 wt %) water.
A98. The composition, method, product, or use of any of A1-A17, wherein the composition consists of or consists essentially of 0.1-3 wt % (preferably 0.5-1.5 wt %) curcumin, 35-85 wt % (preferably 55-65 wt %) of the one or more glyceride(s), and 15-65 wt % (preferably 35-45 wt %) water.
A99. The composition, method, product, or use of any of A1-A17, wherein the composition comprises 0.1-3 wt % curcumin, 35-85 wt % glyceryl monooleate, and 15-65 wt % water.
A100. The composition, method, product, or use of any of A1-A17, wherein the composition comprises 0.5-1.5 wt % curcumin, 55-65 wt % glyceryl monooleate, and 35-45 wt % water.
A101. The composition, method, product, or use of any of A1-A17, wherein the composition consists of or consists essentially of 0.5-1.5 wt % curcumin, 55-65 wt % glyceryl monooleate, and 35-45 wt % water.
A102. The composition, method, product, or use of any of A1-A17, wherein the composition comprises 0.5-5 wt % (preferably 1-2 wt %) curcumin compound, 45-75 wt % (preferably 55-65 wt %) of the one or more (preferably at least two, preferably two) glyceride(s), and 25-55 wt % (preferably 35-45 wt %) water.
A103. The composition, method, product, or use of any of A1-A17, wherein the composition consists of or consists essentially of 0.5-5 wt % (preferably 1-2 wt %) curcumin compound, 45-75 wt % (preferably 55-65 wt %) of the one or more (preferably at least two, preferably two) glyceride(s), and 25-55 wt % (preferably 35-45 wt %) water.
A104. The composition, method, product, or use of any of A1-A17, wherein the composition comprises 0.5-5 wt % (preferably 1-2 wt %) curcumin, 45-75 wt % (preferably 55-65 wt %) of the one or more (preferably at least two, preferably two) glyceride(s), and 25-55 wt % (preferably 35-45 wt %) water.
A105. The composition, method, product, or use of any of A1-A17, wherein the composition consists of or consists essentially of 0.5-5 wt % (preferably 1-2 wt %) curcumin, 45-75 wt % (preferably 55-65 wt %) of the one or more (preferably at least two, preferably two) glyceride(s), and 25-55 wt % (preferably 35-45 wt %) water.
A106. The composition, method, product, or use of any of A1-A17, wherein the composition comprises 0.5-5 wt % (preferably 1-2 wt %) curcumin, 45-75 wt % (preferably 55-65 wt %) mixture of glyceryl monooleate and glyceryl monolinoleate in a respective weight ratio between 25:75 and 35:65, and 25-55 wt % (preferably 35-45 wt %) water.
A107. The composition, method, product, or use of any of A1-A17, wherein the composition consists of or consists essentially of 0.5-5 wt % (preferably 1-2 wt %) curcumin, 45-75 wt % (preferably 55-65 wt %) mixture of glyceryl monooleate and glyceryl monolinoleate in a respective weight ratio between 25:75 and 35:65, and 25-55 wt % (preferably 35-45 wt %) water.
A108. The composition, method, product, or use of any of A1-A17, wherein the composition comprises 10-30 wt % (preferably 17.5-22.5 wt %) curcumin compound, 30-70 wt % (preferably 45-55 wt %) of the one or more (preferably at least two, preferably two) glyceride(s), and 10-50 wt % (preferably 25-35 wt %) water.
A109. The composition, method, product, or use of any of A1-A17, wherein the composition consists of or consists essentially of 10-30 wt % (preferably 17.5-22.5 wt %) curcumin compound, 30-70 wt % (preferably 45-55 wt %) of the one or more (preferably at least two, preferably two) glyceride(s), and 10-50 wt % (preferably 25-35 wt %) water.
A110. The composition, method, product, or use of any of A1-A17, wherein the composition comprises 10-30 wt % (preferably 17.5-22.5 wt %) curcumin, 30-70 wt % (preferably 45-55 wt %) of the one or more (preferably at least two, preferably two) glyceride(s), and 10-50 wt % (preferably 25-35 wt %) water.
A111. The composition, method, product, or use of any of A1-A17, wherein the composition consists of or consists essentially of 10-30 wt % (preferably 17.5-22.5 wt %) curcumin, 30-70 wt % (preferably 45-55 wt %) of the one or more (preferably at least two, preferably two) glyceride(s), and 10-50 wt % (preferably 25-35 wt %) water.
A112. The composition, method, product, or use of any of A1-A17, wherein the composition comprises 10-30 wt % (preferably 17.5-22.5 wt %) curcumin, 30-70 wt % (preferably 45-55 wt %) mixture of glyceryl monooleate and glyceryl monolinoleate in a respective weight ratio between 25:75 and 35:65, and 10-50 wt % (preferably 25-35 wt %) water.
A113. The composition, method, product, or use of any of A1-A17, wherein the composition consists of or consists essentially of 10-30 wt % (preferably 17.5-22.5 wt %) curcumin, 30-70 wt % (preferably 45-55 wt %) mixture of glyceryl monooleate and glyceryl monolinoleate in a respective weight ratio between 25:75 and 35:65, and 10-50 wt % (preferably 25-35 wt %) water.
A114. The composition, method, product, or use of any of A1-A17, wherein the composition comprises 0.005-0.5 wt % (preferably 0.01-0.1 wt %) curcumin compound, 1-10 wt % (preferably 3-7 wt %) of the one or more (preferably at least two, preferably two) glyceride(s), and 90-99 wt % (preferably 93-97 wt %) water.
A115. The composition, method, product, or use of any of A1-A17, wherein the composition comprises 0.005-0.5 wt % (preferably 0.01-0.1 wt %) curcumin compound, 1-10 wt % (preferably 3-7 wt %) of the one or more (preferably at least two, preferably two) glyceride(s), 90-99 wt % (preferably 93-97 wt %) water, and 0.01-2 wt % (preferably 0.1-1 wt %) surfactant (preferably a surfactant as defined herein).
A116. The composition, method, product, or use of any of A1-A17, wherein the composition consists of or consists essentially of 0.005-0.5 wt % (preferably 0.01-0.1 wt %) curcumin compound, 1-10 wt % (preferably 3-7 wt %) of the one or more (preferably at least two, preferably two) glyceride(s), 90-99 wt % (preferably 93-97 wt %) water, and 0.01-2 wt % (preferably 0.1-1 wt %) surfactant (preferably a surfactant as defined herein).
A117. The composition, method, product, or use of any of A1-A17, wherein the composition comprises 0.005-0.5 wt % (preferably 0.01-0.1 wt %) curcumin, 1-10 wt % (preferably 3-7 wt %) of the one or more (preferably at least two, preferably two) glyceride(s), and 90-99 wt % (preferably 93-97 wt %) water.
A118. The composition, method, product, or use of any of A1-A17, wherein the composition comprises 0.005-0.5 wt % (preferably 0.01-0.1 wt %) curcumin, 1-10 wt % (preferably 3-7 wt %) of the one or more (preferably at least two, preferably two) glyceride(s), 90-99 wt % (preferably 93-97 wt %) water, and 0.01-2 wt % (preferably 0.1-1 wt %) surfactant (preferably a surfactant as defined herein).
A119. The composition, method, product, or use of any of A1-A17, wherein the composition consists of or consists essentially of 0.005-0.5 wt % (preferably 0.01-0.1 wt %) curcumin, 1-10 wt % (preferably 3-7 wt %) of the one or more (preferably at least two, preferably two) glyceride(s), 90-99 wt % (preferably 93-97 wt %) water, and 0.01-2 wt % (preferably 0.1-1 wt %) surfactant (preferably a surfactant as defined herein).
A120. The composition, method, product, or use of any of A1-A17, wherein the composition comprises 0.005-0.5 wt % (preferably 0.01-0.1 wt %) curcumin, 1-10 wt % (preferably 3-7 wt %) mixture of glyceryl monooleate and glyceryl monolinoleate in a respective weight ratio between 25:75 and 35:65, and 90-99 wt % (preferably 93-97 wt %) water.
A121. The composition, method, product, or use of any of A1-A17, wherein the composition comprises 0.005-0.5 wt % (preferably 0.01-0.1 wt %) curcumin, 1-10 wt % (preferably 3-7 wt %) mixture of glyceryl monooleate and glyceryl monolinoleate in a respective weight ratio between 25:75 and 35:65, 90-99 wt % (preferably 93-97 wt %) water, and 0.01-2 wt % (preferably 0.1-1 wt %) surfactant (preferably a surfactant as defined herein).
A122. The composition, method, product, or use of any of A1-A17, wherein the composition consists of or consists essentially of 0.005-0.5 wt % (preferably 0.01-0.1 wt %) curcumin, 1-10 wt % (preferably 3-7 wt %) mixture of glyceryl monooleate and glyceryl monolinoleate in a respective weight ratio between 25:75 and 35:65, 90-99 wt % (preferably 93-97 wt %) water, and 0.01-2 wt % (preferably 0.1-1 wt %) surfactant (preferably a surfactant as defined herein).
A123. The composition, method, product, or use of any of A1-A17, wherein the composition comprises 0.1-3 wt % (preferably 0.5-1.7 wt %) curcumin compound, 5-25 wt % (preferably 12-18 wt %) of the one or more (preferably at least two, preferably two) glyceride(s), and 75-90 wt % (preferably 80-88 wt %) water.
A124. The composition, method, product, or use of any of A1-A17, wherein the composition comprises 0.1-3 wt % (preferably 0.5-1.7 wt %) curcumin compound, 5-25 wt % (preferably 12-18 wt %) of the one or more (preferably at least two, preferably two) glyceride(s), 75-90 wt % (preferably 80-88 wt %) water, and 0.01-2 wt % (preferably 0.1-1 wt %) surfactant (preferably a surfactant as defined herein).
A125. The composition, method, product, or use of any of A1-A17, wherein the composition consists of or consists essentially of 0.1-3 wt % (preferably 0.5-1.7 wt %) curcumin compound, 5-25 wt % (preferably 12-18 wt %) of the one or more (preferably at least two, preferably two) glyceride(s), 75-90 wt % (preferably 80-88 wt %) water, and 0.01-2 wt % (preferably 0.1-1 wt %) surfactant (preferably a surfactant as defined herein).
A126. The composition, method, product, or use of any of A1-A17, wherein the composition comprises 0.1-3 wt % (preferably 0.5-1.7 wt %) curcumin, 5-25 wt % (preferably 12-18 wt %) of the one or more (preferably at least two, preferably two) glyceride(s), and 75-90 wt % (preferably 80-88 wt %) water.
A127. The composition, method, product, or use of any of A1-A17, wherein the composition comprises 0.1-3 wt % (preferably 0.5-1.7 wt %) curcumin, 5-25 wt % (preferably 12-18 wt %) of the one or more (preferably at least two, preferably two) glyceride(s), 75-90 wt % (preferably 80-88 wt %) water, and 0.01-2 wt % (preferably 0.1-1 wt %) surfactant (preferably a surfactant as defined herein).
A128. The composition, method, product, or use of any of A1-A17, wherein the composition consists of or consists essentially of 0.1-3 wt % (preferably 0.5-1.7 wt %) curcumin, 5-25 wt % (preferably 12-18 wt %) of the one or more (preferably at least two, preferably two) glyceride(s), 75-90 wt % (preferably 80-88 wt %) water, and 0.01-2 wt % (preferably 0.1-1 wt %) surfactant (preferably a surfactant as defined herein).
A129. The composition, method, product, or use of any of A1-A17, wherein the composition comprises 0.1-3 wt % (preferably 0.5-1.7 wt %) curcumin, 5-25 wt % (preferably 12-18 wt %) mixture of glyceryl monooleate and glyceryl monolinoleate in a respective weight ratio between 25:75 and 35:65, and 75-90 wt % (preferably 80-88 wt %) water.
A130. The composition, method, product, or use of any of A1-A17, wherein the composition comprises 0.1-3 wt % (preferably 0.5-1.7 wt %) curcumin, 5-25 wt % (preferably 12-18 wt %) mixture of glyceryl monooleate and glyceryl monolinoleate in a respective weight ratio between 25:75 and 35:65, 75-90 wt % (preferably 80-88 wt %) water, and 0.01-2 wt % (preferably 0.1-1 wt %) surfactant (preferably a surfactant as defined herein).
A131. The composition, method, product, or use of any of A1-A17, wherein the composition consists of or consists essentially of 0.1-3 wt % (preferably 0.5-1.7 wt %) curcumin, 5-25 wt % (preferably 12-18 wt %) mixture of glyceryl monooleate and glyceryl monolinoleate in a respective weight ratio between 25:75 and 35:65, 75-90 wt % (preferably 80-88 wt %) water, and 0.01-2 wt % (preferably 0.1-1 wt %) surfactant (preferably a surfactant as defined herein).

Specific embodiments A1-A131, and features thereof, may be further defined and/or sub-defined as set forth herein, in particular (and where compatible in the given context) as set forth in paragraphs B1-B450 below. In particular, any composition or product of any of specific embodiments A1-A131 may be a composition or product as defined in any of B2-B43. Any definition pertaining to a composition are equally applicable to methods, products, and uses of the invention, albeit, in preparative methods, ingredients of the compositions will generally be pertinent in the context of them being mixed together or otherwise combined or incorporated to provide relevant products of said methods (be them compositions or other products).

### Specific Embodiments Via Dependency

The following numbered paragraphs (B1-B450), with dependencies, describe further embodiments and features which may be combined with any embodiments and features disclosed in any other section herein, including the embodiments of A1-A131. A person skilled in the art will readily appreciate embodiments elucidated by the dependencies set forth below, including via direct and indirect dependencies.

### B) Numbered Paragraphs with Dependencies

B1. A composition, either in general or suitably as defined anywhere herein, including as defined in any of paragraphs A1-A131 above (in particular where compatible in the given context).
B2. The composition of B1, wherein the composition is a topical composition (e.g. for use on a bodily surface, for example skin, eye(s), body cavity, and/or hair).
B3. The composition of B2, wherein the topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.
B4. The composition of B3, wherein the topical composition is a cream, an ointment, a foam, or a perfume (e.g. perfume, aftershave, cologne, or eau de toilette).
B5. The composition of B4, wherein the topical composition is a cream.
B6. The composition of B4, wherein the topical composition is an ointment.
B7. The composition of B2, wherein the composition is an oral topical composition, for example, a mouthwash, a rinse, an oral spray, a suspension, and/or a dental gel, suitably which is intended to be taken by mouth but are not intended to be ingested.
B8. The composition of B1, wherein composition is an ingestible composition (e.g. orally-adminstrable composition).
B9. The composition of B8, wherein the ingestible composition is selected from a lozenge, a gel, a jelly, a pastille, a tablet, a capsule (e.g. a capsule containing the composition), a toffee, a nougat, a chewy candy, and/or a chewing gum.
B110. The composition of B9, wherein the ingestible composition is (or is otherwise incorporated within) a pastille, a gel, or a jelly.
B111. The composition of B8-B110, wherein the ingestible composition comprises a gel component, gelling component, or gellable component.
B112. The composition of B111, wherein the gel component, gelling component, or gellable component is animal- or plant-derived.
B113. The composition of B112, wherein the gel component, gelling component, or gellable component is plant-derived (suitably the composition or product into which said composition is incorporated is free of any animal-derived components/ingredients).
B14. The composition of B11-B13, wherein the gel component, gelling component, or gellable component comprises or consists of a polysaccharide or derivative thereof (e.g. x-carrageenan).
B15. The composition of B11-B14, wherein the gel component, gelling component, or gellable component comprises or consists of a gelatin, agar, and/or agarose.
B116. The composition of B8, wherein the ingestible composition is (or is otherwise incorporated within, for instance, by mixing the composition with a solvent such as water) a liquid, suitably a suspension, a dispersion, or a solution.
B17. The composition of B16, wherein the ingestible composition is a drink/beverage. The drink/beverage may be an alcoholic or non-alcoholic drink/beverage.
B18. The composition of B1-B17, wherein the composition is (or is otherwise incorporated within) a pharmaceutical composition.
B19. The composition of B18, wherein the pharmaceutical composition is for use as a medicament (or may otherwise be used in the manufacture of a medicament).
B20. The composition of B118, wherein the pharmaceutical composition is (or is otherwise incorporated within a product that is) an ayurvedic medicine.
B21. The composition of B18, wherein the pharmaceutical composition is for (or is for use in) treating: neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukaemia, and colorectal cancer), anaphylaxis, arthritis, osteoarthritis, acne, and/or eczema.
B22. The composition of B1-B17, wherein the composition is (or is otherwise incorporated within) a nutraceutical composition.
B23. The composition of B1-B17, wherein the composition is (or is otherwise incorporated within) a food supplement.
B24. The composition of B1-B17, wherein the composition is (or is otherwise incorporated within) a food additive.
B25. The composition of B1-B17, wherein the composition is (or is otherwise incorporated within) a foodstuff.
B26. The composition of B1-B17, wherein the composition is not a foodstuff or a food additive.
B27. The composition of B1-B17, wherein the composition is not a food supplement, food additive, or a foodstuff.
B28. The composition of B1-B17, wherein the composition is a personal care composition.
B29. The composition of B28, wherein the personal care composition is selected from the group consisting of skin care compositions, skin creams, shaving compositions (e.g. shaving creams, gels, foams), moisturizers, lotions, body washes, body oils, and hair care compositions (e.g. shampoos and/or hair conditioners).
B30. The composition of B28-B29, wherein the composition is a “leave-on-type” composition (e.g. moisturizing lotions, serums, creams) or a “rinse-off-type” composition (e.g. body washes, shampoos, hair conditioners, shower gels, skin cleansers, cleansing milks, shaving compositions).
B31. The composition of B11-B30 (especially B28-B30), wherein the composition is in the form of (or is otherwise incorporated within) an emulsion, a lotion, a milk, a liquid, a solid, a cream, a gel, a mousse, an ointment, a paste, a serum, a stick, a spray, a tonic, an aerosol, a foam, and/or a pencil.
B32. The composition of B1-B31 (especially B28-B31), wherein the composition is in the form of (or is otherwise incorporated within) an implement (e.g. a powdered or unpowdered cosmetic applicator), a reusable or disposable wipe, a tissue, a towel, a diaper, a razor or other shaving device, a personal cleansing implement (e.g. a mesh shower sponge), aconventional sponge, a wash cloth, a swab, and/or a pen.
B33. The composition of B1-B32, wherein the composition is a cosmetic composition (or is a cosmetic product or is incorporated within a cosmetic product—e.g. a cosmetic product comprising the composition).
B34. The composition of B33, wherein the cosmetic composition is for controlling and/or reducing the formation of wrinkles and lines in the skin.
B35. The composition of B33, wherein the cosmetic composition is for providing glowing skin.
B36. The composition of B11-B117, wherein the composition is a wound care composition (or is a wound care product or is incorporated within a wound care product—e.g. a wound care product comprising the composition).
B37. The composition of B36, wherein the wound care (or wound healing) composition is a wound care hydrogel composition, and therefor suitable comprises a hydrogel or hydrogel-forming component or compound.
B38. The composition of B36-B37, wherein the wound care composition is incorporated within a wound care product—e.g. a wound care product comprising the composition.
B39. The composition of B38, wherein wound care product is selected from the group consisting of a wound dressing, a sutures, a staple, a gauze, a bandage, a plaster, a burn dressing, an artificial skin, a liposome or micell formulation, a microcapsule, an aqueous vehicle for soaking gauze dressings.
B40. The composition of B36-B39, wherein the composition is for (or for use in) healing wounds, for example, healing cuts and scrapes, burns, ulcers, bed sores, fissures, haemorrhoids, and/or post-surgical wounds.
B41. The composition of B1-B17, wherein the composition is (or is otherwise incorporated within) a textile.
B42. The composition of B1-B41, wherein the composition is an oil-in-water dispersion.
B43. The composition of B1-B41, wherein the composition is an water-in-oil dispersion.

### Curcumin Compound

B44. The composition of B1-B43, comprising a curcumin compound.
B45. The composition of B1-B44, wherein the curcumin compound is curcumin (or a derivative or an analogue thereof) or a pharmaceutically acceptable salt thereof.
B46. The composition of B44-B45, wherein the curcumin compound is defined by Formula I:

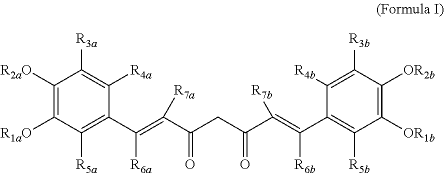

wherein:
   each R₁ₐ, R_{1b}, R₂ₐ, and R_{2b} group is independently selected from any R_{OX} group, and/or either or both pairs of R₁ₐ/R₂ₐ and/or R_{1b}/R_{2b} are linked together to form an optionally substituted heterocyclic or heteroaryl ring; and
   each R₃ₐ, R_{3b}, R₄ₐ, R_{4b}, R₅ₐ, R_{5b}, R₆ₐ, R_{6b}, R₇ₐ, and R_{7b} group is independently selected from any R_{CAR} group, and/or either or both pairs of R₃ₐ/R₄ₐ and/or R_{3b}/R_{4b} are linked together to form an optionally substituted cycloalkyl, cycloalkenyl, heterocyclic, or heteroaryl ring;
wherein:
   each R_{OX} group is selected from hydrogen, trifluoromethyl, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N,N-di-[(1-6C)alkyl]sulphamoyl, or from a group of the formula:

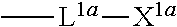

wherein:
L^{1a} is absent or is selected from SO, SO₂, CO, CH(OR_{A}), CON(R_{A}), N(R_{A})CO, SO₂N(R_{A}), wherein R_{A} is hydrogen or (1-8C)alkyl; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{OX} group is optionally further substituted with one or more R_{CAR} groups as defined below; each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

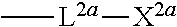

wherein:
L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above; wherein optionally either or both OR₁ₐ and/or OR_{1b} are replaced by hydrogen (e.g. dealkoxy analogues);
or any pharmaceutically acceptable salt thereof.
B47. The composition of B46, wherein each R_{OX} group is selected from hydrogen, (1-8C)alkyl, (2-6C)alkanoyl, wherein any R_{OX} group is optionally further substituted with one or more R_{CAR} groups.
B48. The composition of B46, wherein each R_{OX} group is selected from hydrogen and (1-3C)alkyl.
B49. The composition of B46, wherein each R_{OX} group is selected from hydrogen and methyl.
B50. The composition of B46-B49, wherein both R₁ₐ and R_{1b} are the same as each other, and both R₂ₐ and R_{2b} are the same as each other, though suitably R₁ₐ/R_{1b} is different to R_{2a/2b}.
B51. The composition of B46-B49, wherein both R₁ₐ and R_{1b} are methyl, and both R₂ₐ and R_{2b} are hydrogen.
B52. The composition of B46-B51, wherein either or both OR₁ₐ and/or OR_{1b} are replaced by hydrogen (e.g. dealkoxy analogues).
B53. The composition of B46-B52, wherein each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (1-8C)hydroxyalkyl, (1-6C)alkoxy, (1-6C)alkylamino, (1-6C)dialkylamino, (2-6C)alkanoyl, or from a group of the formula:

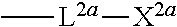

wherein:
   L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
   X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above.
B54. The composition of B53, wherein each R_{CAR} group is selected from hydrogen, halogeno, trifluoromethyl, cyano, hydroxy, amino, carboxy, (1-8C)alkyl, (1-8C)hydroxyalkyl, (1-6C)alkoxy, (2-6C)alkanoyl.
B55. The composition of B54, wherein each R_{CAR} group is selected from hydrogen, fluoro, or methyl.
B56. The composition of B55, wherein each R_{CAR} group is hydrogen.
B57. The composition of B46-B56, wherein both R₃ₐ and R_{3b} are the same as each other, both R₄ₐ and R_{4b} are the same as each other, both R₅ₐ and R_{5b} are the same as each other, both R₆ₐ and R_{6b} are the same as each other, and both R₇ₐ and R_{7b} are the same as each other.
B58. The composition of B46-B57, wherein each R₃ₐ, R_{3b}, R₄ₐ, R_{4b}, R₅ₐ, R_{5b}, R₆ₐ, R_{6b}, R₇ₐ, and R_{7b} group is hydrogen.
B59. The composition of B46-B58, wherein the curcumin compound is defined by Formula II:

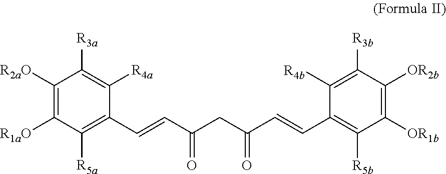

wherein R₁ₐ, R_{1b}, R₂ₐ, R_{2b}, R₃ₐ, R_{3b}, R₄ₐ, R_{4b}, R₅ₐ, and R_{5b}, are as defined in any of B46-B58;
wherein optionally either or both OR₁ₐ and/or OR_{1b} are replaced by hydrogen (e.g. dealkoxy analogues);
or any pharmaceutically acceptable salt thereof.
B60. The composition of B46-B59, wherein the curcumin compound is defined by Formula III:

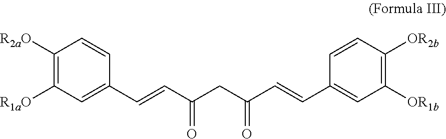

wherein R₁ₐ, R_{1b}, R₂ₐ, and R_{2b}, are as defined in any of B46-B59;
wherein optionally either or both OR₁ₐ and/or OR_{1b} are replaced by hydrogen (e.g. dealkoxy analogues);
or any pharmaceutically acceptable salt thereof.
B61. The composition of B46-B60, wherein the curcumin compound is defined by Formula III:

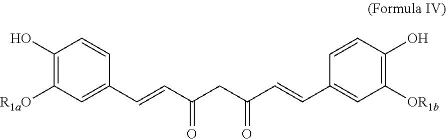

wherein R₁ₐ, and R_{1b}, are as defined in any of B46-B60;
wherein optionally either or both OR₁ₐ and/or OR_{1b} are replaced by hydrogen (e.g. dealkoxy analogues);
or any pharmaceutically acceptable salt thereof.
B62. The composition of B46-B60, wherein the curcumin compound is defined by Formula IV:

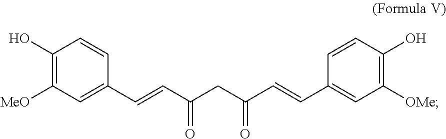 [SMILES: COc1cc(/C=C/C(=O)CC(=O)/C=C/c2ccc(O)c(OC)c2)ccc1O]

wherein optionally either or both OMe groups are replaced by hydrogen (e.g. dimethoxy or bisdemethoxy analogues);
or any pharmaceutically acceptable salt thereof.
B63. The composition of B44, wherein the curcumin compound is curcumin or a pharmaceutically acceptable salt thereof.
B64. The composition of B44, wherein the curcumin compound is curcumin (suitably unsalted curcumin).
B65. The composition of B44, wherein the curcumin compound is a metabolite of curcumin, for example a curcumin glucuronide.
B66. The composition of B44, wherein the curcumin compound is demethoxycurcumin or a salt thereof (suitably unsalted demethoxycurcumin).
B67. The composition of B44, wherein the curcumin compound is bisdemethoxycurcumin or a salt thereof (suitably unsalted bisdemethoxycurcumin).
B68. The composition of B44-B67, wherein the curcumin compound is substantially pure curcumin compound (e.g. greater than or equal to 95 wt % pure, suitably greater than or equal to 98 wt % pure, preferably greater than or equal to 99 wt % pure).
B69. The composition of B68, wherein the composition is substantially free of turmeric or comprises turmeric compounds (i.e. compounds within turmeric other than the curcumin compound) at a concentration of at most 5 wt %, suitably at most 2 wt %, preferably at most 1 wt %.
B70. The composition of any of B1-B67, wherein the curcumin compound is provided to the composition by (or as) a curcumin compound source which comprises the (main) curcumin compound in admixture with one or more other source compounds (e.g. one or more other curcumin compounds, and possibly other ingredients/components). These “other source compounds” may include one or more other curcumin compounds (e.g. curcuminoids), one or more turmerones, and such like.
B71. The composition of B70, wherein the curcumin compound source is turmeric, a turmeric extract, and/or a curcuminoids mixture.
B72. The composition of B70, wherein the curcumin compound source is turmeric.
B73. The composition of B70, wherein the curcumin compound source is a turmeric extract.
B74. The composition of B70, wherein the curcumin compound source is a curcuminoids mixture.
B75. The composition of B70-B74, wherein the (main) curcumin compound is, notwithstanding the presence of “other source compounds” (e.g. other turmeric compounds), present within the composition in an amount, concentration (e.g. as defined in B285-B349), or ratio (e.g. as defined in B350-B376), as defined herein.
B76. The composition of any of B1-B75, wherein the composition comprises one or more curcumin compounds, wherein each of the one or more curcumin compounds is independently a curcumin compound as defined in any of B44-B75. However, references within this specification to “a curcumin compound” or “the curcumin compound” preferably relates to a single (primary, i.e. “main”) curcumin compound (most preferably curcumin itself), even though other curcumin compounds may also be present.
B77. The composition of any of B1-B76, wherein the composition comprises one or more curcumin compounds, one of which is a “main” curcumin compound (preferably as defined in any of B44-B75, most suitably curcumin itself) and the other(s) of which is/are (one or more) other curcumin compound(s) (each of which may also be as defined in any of B44-B75, so long as each is different from the main curcumin compound). Preferably, the main curcumin compound is, notwithstanding the presence of (one or more) other curcumin compound(s), present within the composition in an amount, concentration (e.g. as defined in B285-B349), or ratio (e.g. as defined in B350-B376), as defined herein.
B78. The composition of B76-B77, wherein at least one of the one or more curcumin compounds is curcumin (or a pharmaceutically acceptable salt thereof).
B79. The composition of B76-B78, wherein curcumin is the main curcumin compound, preferably in that the curcumin concentration (e.g. as a wt % within the composition or as a ratio with any other component), is higher than any other curcumin compound.
B80. The composition of B78 or B79, wherein the curcumin (or pharmaceutically acceptable salt thereof) constitutes between 30 wt % and 99.999 wt % of all curcumin compound(s) present within the composition.
B81. The composition of B78-B80, wherein the curcumin (or pharmaceutically acceptable salt thereof) constitutes greater than or equal to 40 wt % of all curcumin compound(s) present within the composition.
B82. The composition of B78-B80, wherein the curcumin (or pharmaceutically acceptable salt thereof) constitutes greater than or equal to 50 wt % of all curcumin compound(s) present within the composition.
B83. The composition of B78-B80, wherein the curcumin (or pharmaceutically acceptable salt thereof) constitutes greater than or equal to 60 wt % of all curcumin compound(s) present within the composition.
B84. The composition of B78-B80, wherein the curcumin (or pharmaceutically acceptable salt thereof) constitutes greater than or equal to 70 wt % of all curcumin compound(s) present within the composition.
B85. The composition of B78-B80, wherein the curcumin (or pharmaceutically acceptable salt thereof) constitutes greater than or equal to 80 wt % of all curcumin compound(s) present within the composition.
B86. The composition of B78-B85, wherein the curcumin (or pharmaceutically acceptable salt thereof) constitutes between 30 wt % and 90 wt % of all curcumin compound(s) present within the composition.

### Glycerides

B87. The composition of B1-B86, wherein the composition comprises one or more glycerides.
B88. The composition of B87, wherein the composition comprises a glyceride.
B89. The composition of B87, wherein the composition comprises a mono- and/or di-glyceride.
B90. The composition of B87, wherein the composition comprises a monoglyceride.
B91. The composition of B87, wherein the composition comprises two or more glycerides.
B92. The composition of B87, wherein the composition comprises at most two glycerides.
B93. The composition of B87, wherein the composition comprises a first glyceride and a second glyceride.
B94. The composition of B87, wherein the composition comprises a first glyceride and a second glyceride in a weight ratio between 1:99 and 99:1.
B95. The composition of B87, wherein the composition comprises a first glyceride and a second glyceride in a weight ratio between 1:99 and 90:10.
B96. The composition of B87, wherein the composition comprises a first glyceride and a second glyceride in a weight ratio between 10:90 and 90:10.
B97. The composition of B87, wherein the composition comprises a first glyceride and a second glyceride in a weight ratio between 10:90 and 50:50.
B98. The composition of B87, wherein the composition comprises a first glyceride and a second glyceride in a weight ratio between 20:80 and 40:60.
B99. The composition of B87, wherein the composition comprises a first glyceride and a second glyceride in a weight ratio between 25:75 and 35:65.
B100. The composition of B87, wherein the composition comprises a first glyceride and a second glyceride in a weight ratio between 27:73 and 30:70.
B101. The composition of B87, wherein the composition comprises one and at most one glycerides.
B102. The composition of B87, wherein the composition comprises two and at most two glycerides.
B103. The composition of B87-B102, wherein the glyceride(s) is/are mono- and/or di-glyceride(s).
B104. The composition of B87-B102, wherein the glyceride(s) is/are mono-glyceride(s).
B105. The composition of B87-B104, wherein at least one of the glyceride(s) has a melting point of 25-55° C.
B106. The composition of B87-B104, wherein at least two (preferably the two which are mentioned/defined) of the glyceride(s) have a melting point of 25-55° C.
B107. The composition of B87-B104, wherein each defined glyceride(s) each has a melting point of 25-55° C.
B108. The composition of B87-B104, wherein at least one of the glyceride(s) has a melting point of 30-45° C.
B109. The composition of B87-B104, wherein at least two (preferably the two which are mentioned/defined) of the glyceride(s) have a melting point of 30-45° C.
B110. The composition of B87-B104, wherein each defined glyceride(s) each has a melting point of 30-45° C.
B111. The composition of B87-B104, wherein at least one of the glyceride(s) has a melting point of 34-41° C.
B112. The composition of B87-B104, wherein at least two (preferably the two which are mentioned/defined) of the glyceride(s) have a melting point of 34-41° C.
B113. The composition of B87-B104, wherein each defined glyceride(s) each has a melting point of 34-41° C.

### Moieties on Glycerides

B114. The composition of B87-B113, wherein the, each, or at least one of the, glyceride(s) comprises one or more unsaturated moieties.
B115. The composition of B114, wherein the, each, or at least one of the, glyceride(s) comprises between one and two unsaturated moieties.
B116. The composition of B114-B115, wherein the, each, or at least one of the, glyceride(s) comprises one (i.e. a single, one and only one) unsaturated moiety.
B117. The composition of B114-B116, wherein the, each, or at least one of the, glyceride(s) comprises two (i.e. two and only two) unsaturated moieties.
B118. The composition of B114-B117, wherein the composition comprises two glycerides, a first glyceride with one (i.e. one and only one) unsaturated moiety and a second glyceride with two (i.e. two and only two) unsaturated moieties. Suitably the respective first and second glyceride may be present in one of the aforementioned weight ratios.
B119. The composition of B114-B118, wherein the, each, or at least one of the, unsaturated moiety(ies) is/are an alkene moiety(ies).
B120. The composition of B119, wherein the, each, or at least one of the, alkene moiety(ies) is/are cis-alkene moiety(ies).
B121. The composition of B87-B120, wherein the, each, or at least one of the, glyceride(s) comprises one, two, or three fatty acid(s) (suitably respectively comprises one or two fatty acids in the case of mono- and/or diglyceride, and suitably respectively comprises only one fatty acid in the case of mono-glycerides) condensed with glycerol.
B122. The composition of B121, wherein the, each, or at least one of the, fatty acid(s) is/are aliphatic fatty acid(s).
B123. The composition of B121, wherein the, each, or at least one of the, fatty acid(s) is/are linear (i.e. unbranched) fatty acid(s).
B124. The composition of B121-B123, wherein the, each, or at least one of the, fatty acid(s) is/are C₆-C₃₀ fatty acid(s) (i.e. having a carbon chain that is 6-30 carbons in length, including the carboxylate carbon).
B125. The composition of B121-B124, wherein the, each, or at least one of the, fatty acid(s) is/are C₈-C₂₀ fatty acid(s).
B126. The composition of B121-B125, wherein the, each, or at least one of the, fatty acid(s) is/are C₁₈ fatty acid(s).
B127. The composition of B121-B126, wherein the, each, or at least one of the, fatty acid(s) comprises one or more unsaturated moieties.
B128. The composition of B127, wherein the, each, or at least one of the, fatty acid(s) comprises between one and two unsaturated moieties.
B129. The composition of B127-B128, wherein the, each, or at least one of the, fatty acid(s) comprises one (i.e. a single, one and only one) unsaturated moiety.
B130. The composition of B127-B129, wherein the, each, or at least one of the, fatty acid(s) comprises two (i.e. two and only two) unsaturated moieties.
B131. The composition of B127-B130, wherein the composition comprises a first glyceride comprising a fatty acid (suitably only a single fatty acid, as per a mono-glyceride) with one or more unsaturated moiety(ies), and a second glyceride comprising a fatty acid (suitably only a single fatty acid, as per a mono-glyceride) with one or more unsaturated moiety(ies).
B132. The composition of B127-B131, wherein the composition comprises a first glyceride comprising a fatty acid (suitably only a single fatty acid, as per a mono-glyceride) with one (i.e. one and only one) unsaturated moiety, and a second glyceride comprising a fatty acid (suitably only a single fatty acid, as per a mono-glyceride) with two (i.e. two and only two) unsaturated moieties.
B133. The composition of B127-B132, wherein the, each, or at least one of the, unsaturated moiety(ies) is/are an alkene moiety(ies).
B134. The composition of B133, wherein the, each, or at least one of the, alkene moiety(ies) is/are cis-alkene moiety(ies).
B135. The composition of B127-B134, wherein the, each, or at least one of the, fatty acid(s) is oleic acid.
B136. The composition of B127-B135, wherein the, each, or at least one of the, fatty acid(s) is linoleic acid.
B137. The composition of B1-B136, wherein the composition comprises glyceryl monooleate (monoolein).
B138. The composition of B1-B137, wherein the composition comprises glyceryl monolinoleate (monolinolein).
B139. The composition of B1-B138, wherein the composition comprises glyceryl monooleate (monoolein) and glyceryl monolinoleate (monolinolein).
B140. The composition of B139, wherein the composition comprises glyceryl monooleate and glyceryl monolinoleate in a weight ratio between 1:99 and 90:10.
B141. The composition of B139, wherein the composition comprises glyceryl monooleate and glyceryl monolinoleate in a weight ratio between 10:90 and 50:50.
B142. The composition of B139, wherein the composition comprises glyceryl monooleate and glyceryl monolinoleate in a weight ratio between 20:80 and 40:60.
B143. The composition of B139, wherein the composition comprises glyceryl monooleate and glyceryl monolinoleate in a weight ratio between 25:75 and 35:65.
B144. The composition of B139, wherein the composition comprises glyceryl monooleate and glyceryl monolinoleate in a weight ratio between 27:73 and 30:70.

### Solvent/Water

B145. The composition of B1-B144, wherein the composition comprises a solvent, preferably water.
B146. The composition of B145, wherein the solvent is a polar solvent.
B147. The composition of B146, wherein the polar solvent is a protic solvent.
B148. The composition of B147, wherein the protic solvent is water.
   Additional Ingredients (or Ingredients which are Limited in Terms of their Presence)
B149. The composition of B1-B148, wherein the composition comprises one or more additional ingredients (or the composition is otherwise characterised by a maximum amount/concentration/ratio of said one or more additional ingredients, in which case the composition may be free of said additional ingredient(s) or comprise a stipulated maximum quantity thereof).
B150. The composition of B149, wherein the one or more additional ingredients comprise one or more ingredients selected from the group consisting of additional active(s), additional lipid(s) (e.g. fatty acid(s)), natural oil(s), bile salt(s) (and/or bile acid(s)), hydrophilic molecule(s), hydrophobic molecule(s), amphiphilic molecule(s), surfactant(s), or any combination thereof.
B151. The composition of B1-B148, wherein, save for active(s) or the curcumin compound, the composition consists of only GRAS grade components.

### Additional Lipids (or Glycerides)

B152. The composition of B149-B151, wherein the one or more additional ingredients comprise one or more additional lipids.
B153. The composition of B152, wherein the one or more additional lipids comprises one or more glyceride(s) other than those stipulated (e.g. those stipulated in B87-B144), suitably where said one or more glyceride(s) other than those stipulated may be referred to as “unstipulated” glyceride(s).
B154. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 50:50 (i.e. the weight ratio is less than or equal to 50:50).
B155. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 30:70 (i.e. the weight ratio is less than or equal to 30:70).
B156. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 20:80 (i.e. the weight ratio is less than or equal to 20:80).
B157. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 15:85 (i.e. the weight ratio is less than or equal to 15:85).
B158. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 10:90 (i.e. the weight ratio is less than or equal to 10:90).
B159. The composition of B153, wherein the weight ratio of unstipulated glyceride(s) (e.g. those other than the glyceride(s) stipulated in B87-B144) to stipulated glyceride(s) (e.g. those stipulated in B87-B144) is between 0:100 to 5:95 (i.e. the weight ratio is less than or equal to 5:95).
B160. The composition of B152, wherein the one or more additional lipids comprises one or more lipid(s) other than stipulated glyceride(s) (e.g. those stipulated in B87-B144), suitably where said one or more lipid(s) other than those stipulated may be referred to as “unstipulated” lipid(s).
B161. The composition of B160, wherein the weight ratio of unstipulated lipid(s) (e.g. lipid(s) other than the glyceride(s) stipulated in B87-B144) to stipulated glyceride(s) (e.g. those stipulated in B87-B144) is between 0:100 to 50:50 (i.e. the weight ratio is less than or equal to 50:50).
B162. The composition of B160, wherein the weight ratio of unstipulated lipid(s) (e.g. lipid(s) other than the glyceride(s) stipulated in B87-B144) to stipulated glyceride(s) (e.g. those stipulated in B87-B144) is between 0:100 to 30:70 (i.e. the weight ratio is less than or equal to 30:70).
B163. The composition of B160, wherein the weight ratio of unstipulated lipid(s) (e.g. lipid(s) those other than the glyceride(s) stipulated in B87-B144) to stipulated glyceride(s) (e.g. those stipulated in B87-B144) is between 0:100 to 20:80 (i.e. the weight ratio is less than or equal to 20:80).
B164. The composition of B160, wherein the weight ratio of unstipulated lipid(s) (e.g. lipid(s) those other than the glyceride(s) stipulated in B87-B144) to stipulated glyceride(s) (e.g. those stipulated in B87-B144) is between 0:100 to 15:85 (i.e. the weight ratio is less than or equal to 15:85).
B165. The composition of B160, wherein the weight ratio of unstipulated lipid(s) (e.g. lipid(s) other than the glyceride(s) stipulated in B87-B144) to stipulated glyceride(s) (e.g. those stipulated in B87-B144) is between 0:100 to 10:90 (i.e. the weight ratio is less than or equal to 10:90).
B166. The composition of B160, wherein the weight ratio of unstipulated lipid(s) (e.g. lipid(s) other than the glyceride(s) stipulated in B87-B144) to stipulated glyceride(s) (e.g. those stipulated in B87-B144) is between 0:100 to 5:95 (i.e. the weight ratio is less than or equal to 5:95).

### Additional Active

B167. The composition of B149-B151, wherein the one or more additional ingredients comprise an additional active ingredient (e.g. in addition to curcumin compound).
B168. The composition of B167, wherein the additional active ingredient is other than a (different) curcumin compound.
B169. The composition of B167-B168, wherein the additional active ingredient is derived from or is a component part of turmeric.
B170. The composition of B167-B169, wherein the additional active ingredient is or comprises a turmerone.
B171. The composition of B149-B170, wherein the composition comprises one or more turmerones.
B172. The composition of B149-B168, wherein the composition is free of turmerones or comprises no more than 10 wt % turmerones.
B173. The composition of B149-B168, wherein the composition is free of turmerones or comprises no more than 1 wt % turmerones.
B174. The composition of B149-B168, wherein the composition is free of turmerones or comprises no more than 0.1 wt % turmerones.
B175. The composition of B149-B168, wherein the composition is free of turmerones or comprises no more than 0.01 wt % turmerones.

### Additional Surfactant

B176. The composition of B149-B175, wherein the one or more additional ingredients comprise a surfactant. Suitably, in the context of the term “surfactant”, this excludes glyceride(s) or at least excludes any glyceride(s) defined/stipulated.
B177. The composition of B176, wherein the surfactant is a non-ionic surfactant.
B178. The composition of B176, wherein the surfactant is a non-ionic surfactant selected from the group consisting of: a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof.
B179. The composition of B176, wherein the surfactant is a non-ionic surfactant selected from the group consisting of: a fatty alcohol, a fatty alcohol ether, a fatty acid ester, a fatty acid amide, alpha-tocopherol, polyglycerol esters, sucrose esters, saponins (e.g. saponin), and any combination thereof.
B180. The composition of B176, wherein the surfactant selected from the group consisting of: polyglycerol esters, sucrose esters, saponins (e.g. saponin), and any combination thereof.
B181. The composition of B176, wherein the surfactant is a saponin (e.g. saponin).
B182. The composition of B176-B181, on the proviso that the surfactant is not a PEG-based surfactant (e.g. the surfactant does not comprise polyethylene glycol polymers or copolymeric blocks of polyethylene glycol).
B183. The composition of B176-B181, on the proviso that the surfactant is not a poloxamer.
B184. The composition of B176-B181, on the proviso that the surfactant is not poloxamer 407.
B185. The composition of B176-B181, on the proviso that the surfactant is not polysorbate 20.
B186. The composition of B176-B181, on the proviso that the surfactant is not PEG-40 castor oil (e.g. not Killiphor RH 40).
B187. The composition of B176-B186, wherein the surfactant is present (suitably where the composition comprises greater than or equal to 45 wt % water) at a concentration of 0.001-10 wt % of the overall composition.
B188. The composition of B176-B186, wherein the surfactant is present (suitably where the composition comprises greater than or equal to 45 wt % water) at a concentration of 0.01-2 wt % of the overall composition.
B189. The composition of B176-B186, wherein the surfactant is present (suitably where the composition comprises greater than or equal to 45 wt % water) at a concentration of 0.1-1.5 wt % of the overall composition.
B190. The composition of B176-B186, wherein the surfactant is present (suitably where the composition comprises greater than or equal to 45 wt % water) at a concentration of 0.4-1 wt % of the overall composition.
B191. The composition of B1-B186, wherein the composition comprises at most 1 wt % surfactant(s), suitably at most 1 wt % of any surfactants defined in any of B176-B186.
B192. The composition of B1-B186, wherein the composition comprises at most 0.1 wt % surfactant(s), suitably at most 0.1 wt % of any surfactants defined in any of B176-B186.
B193. The composition of B1-B186, wherein the composition comprises at most 0.01 wt % surfactant(s), suitably at most 0.01 wt % of any surfactants defined in any of B176-B186.
B194. The composition of B1-B186, wherein the composition comprises at most 0.001 wt % surfactant(s), suitably at most 0.001 wt % of any surfactants defined in any of B176-B186.
B195. The composition of B1-B173, wherein the composition is free of surfactants, suitably free of any surfactants defined in any of B176-B186.

### Additional Oils

B196. The composition of B149-B195, wherein the one or more additional ingredients comprise one or more oils.
B197. The composition of B196, wherein the oils comprise or consist of a natural oil.
B198. The composition of B196-B197, wherein the oils comprise or consist of an organic oil (suitably animal or vegetable oil).
B199. The composition of B198, wherein the organic oil is a vegetable oil (usually rich in mono, di, and/or triglycerides) selected from the group consisting of (or any combination thereof):
Common vegetable oils: Coconut oil, Corn oil, Cottonseed oil, Olive oil, Palm oil, Peanut oil (Ground nut oil), Rapeseed oil (including Canola oil), Safflower oil, Sesame oil, Soybean oil (or soya oil), Sunflower oil;
Nut oils: Almond oil, Beech nut oil, Brazil nut oil, Cashew oil, Hazelnut oil, Macadamia oil, Mongongo nut oil, Pecan oil, Pine nut oil, Pistachio oil, Walnut oil;
Citrus oils: Grapefruit seed oil, Lemon oil, Orange oil,
Cucurbitaceae oils: Bitter gourd oil, Bottle gourd oil, Buffalo gourd oil, Butternut squash seed oil, Egusi seed oil, Pumpkin seed oil, Watermelon seed oil;
Food Supplement oils: Agai oil, Black seed oil, Blackcurrant seed oil, Borage seed oil, Evening primrose oil, Flaxseed oil;
Other Edible oils: Amaranth oil, Apricot oil, Apple seed oil, Argan oil, Avocado oil, Babassu oil, Ben oil, Borneo tallow nut oil, Cape chestnut oil, Carob pod oil (Algaroba oil)Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil (pressed), Niger seed oil, Poppyseed oil, Nutmeg butter, Okra seed oil, Papaya seed oil, Perilla seed oil, Persimmon seed oil, Pequi oil, Pili nut oil, Pomegranate seed oil, Poppyseed oil, Pracaxi oil, Virgin pracaxi oil, Prune kernel oil, Quinoa oil, Ramtil oil, Rice bran oil, Royle oil, Shea nuts, Sacha inchi oil, Sapote oil, Seje oil, Shea butter, Taramira oil, Tea seed oil (Camellia oil), Thistle oil, Tigernut oil (or nut-sedge oil), Tobacco seed oil, Tomato seed oil, Wheat germ oil;
Multi-purpose oils: Castor oil, Coconut oil (copra oil), Colza oil, Corn oil, Cottonseed oil, False flax oil, Hemp oil, Mustard oil, Palm oil, Peanut oil, Radish oil, Rapeseed oil, Ramtil oil, Rice bran oil, Safflower oil, Salicornia oil, Soybean oil (or soya oil), Sunflower oil, Tigernut oil, Tung oil;
Inedible oils: Copaiba, Jatropha oil, Jojoba oil, Milk bush, Nahor oil, Paradise oil, Petroleum nut oil, Pongamia oil (also known as Honge oil);
Drying oils: Dammar oil, Linseed oil, Poppyseed oil, Stillingia oil, Tung oil, Vernonia oil; and
Miscellaneous vegetable oils: Amur cork tree fruit oil, Artichoke oil, Astrocaryum murumuru butter, Balanos oil, Bladderpod oil, Brucea javanica oil, Burdock oil (Bur oil), Buriti oil, Candlenut oil (Kukui nut oil), Carrot seed oil (pressed), Castor oil, Chaulmoogra oil, Crambe oil, Croton oil (tiglium oil), Cuphea oil, Cupuagu butter, Honesty oil, Illipe butter, Jojoba oil, Mango oil, Mowrah butter, Neem oil, Ojon oil, Passion fruit oil, Rose hip seed oil, Rubber seed oil, Sea buckthorn oil, Sea rocket seed oil, Snowball seed oil (Viburnum oil), Tall oil, Tamanu or foraha oil, Tonka bean oil (Cumaru oil), Tucuma butter, Ucuhuba seed oil, moringa oil.
B200. The composition of B199, wherein the organic oil is a moringa oil, coconut oil, sunflower oil, avocado oil, or any combination thereof.
B201. The composition of B199, wherein the organic oil is a moringa oil, coconut oil, sunflower oil, avocado oil, soya oil (or soybean oil), corn oil, or any combination thereof.
B202. The composition of B199, wherein the organic oil is moringa oil.
B203. The composition of B199, wherein the organic oil is coconut oil.
B204. The composition of B199, wherein the organic oil is sunflower oil.
B205. The composition of B199, wherein the organic oil is avocado oil.
B206. The composition of B199, wherein the organic oil is soya oil (or soybean oil).
B207. The composition of B199, wherein the organic oil is corn oil.
B208. The composition of B196-B207, wherein the one or more oils is a single oil.
B209. The composition of B196-B208, wherein the composition comprises 0.1-70 wt % oil(s).
B210. The composition of B196-B208, wherein the composition comprises 1-60 wt % oil(s).
B211. The composition of B196-B208, wherein the composition comprises 2-50 wt % oil(s).
B212. The composition of B196-B208, wherein the composition comprises 1-70 wt % oil(s).
B213. The composition of B196-B208, wherein the composition comprises 2-60 wt % oil(s).
B214. The composition of B196-B208, wherein the composition comprises 3-50 wt % oil(s).
B215. The composition of B196-B208, wherein the composition comprises 1-30 wt % oil(s).
B216. The composition of B196-B208, wherein the composition comprises 2-20 wt % oil(s).
B217. The composition of B196-B208, wherein the composition comprises 3-10 wt % oil(s).
B218. The composition of B196-B208, wherein the composition comprises 1-50 wt % oil(s).
B219. The composition of B196-B208, wherein the composition comprises 2-40 wt % oil(s).
B220. The composition of B196-B208, wherein the composition comprises 3-30 wt % oil(s).
B221. The composition of B196-B220, when dependent upon B78 or any paragraph dependent thereon, wherein the weight ratio of stipulated glyceride(s) to oil(s) is 99:1 to 1:99.
B222. The composition of B196-B221, when dependent upon B87 or any paragraph dependent thereon, wherein the weight ratio of stipulated glyceride(s) to oil(s) is 99:1 to 30:70.
B223. The composition of B222, wherein the weight ratio of stipulated glyceride(s) to oil(s) is 95:5 and 30:70.
B224. The composition of B222, wherein the weight ratio of stipulated glyceride(s) to oil(s) is 95:5 to 50:40.
B225. The composition of B222, wherein the weight ratio of stipulated glyceride(s) to oil(s) is 90:10 to 50:50.
B226. The composition of B222, wherein the weight ratio of stipulated glyceride(s) to oil(s) is 90:10 to 60:40.

### Additional Bile Acids or Bile Salts

B227. The composition of B149-B226, wherein the one or more additional ingredients comprise individual or mixtures of bile acids and/or bile salts.

### Additional Aloe Vera

B228. The composition of B149-B227, wherein the one or more additional ingredients comprise aloe vera, preferably in the form of aloe vera gel. In such embodiments, the composition preferably comprises 0.001-50 wt % curcumin compound, though more preferably 0.01-10 wt % curcumin compound, and most preferably 0.5-5 wt % curcumin compound.
B229. The composition of B228, wherein the composition additionally comprises a gum, preferably xanthan gum.
B230. The composition of B228-B229, wherein the composition additionally comprises glycerol, preferably vegetable glycerol.
B231. The composition of B228-B230, when dependent upon B78 or any paragraph dependent thereon, wherein the weight ratio of stipulated glyceride(s) to aloe vera is 99:1 to 1:99.
B232. The composition of B228-B230, when dependent upon B78 or any paragraph dependent thereon, wherein the weight ratio of stipulated glyceride(s) to aloe vera is 99:1 to 30:70.
B233. The composition of B228-B230, when dependent upon B78 or any paragraph dependent thereon, wherein the weight ratio of stipulated glyceride(s) to aloe vera is 95:5 to 30:70.
B234. The composition of B228-B230, when dependent upon B78 or any paragraph dependent thereon, wherein the weight ratio of stipulated glyceride(s) to aloe vera is 90:10 to 30:70.
B235. The composition of B228-B230, when dependent upon B78 or any paragraph dependent thereon, wherein the weight ratio of stipulated glyceride(s) to aloe vera is 60:40 to 40:60.
B236. The composition of B230 or any preceding paragraph dependent thereon, wherein the gum (preferably xanthan gum) and glycerol are present within the composition in a weight ratio between 10:1 to 1:10.
B237. The composition of B230 or any preceding paragraph dependent thereon, wherein the gum (preferably xanthan gum) and glycerol are present within the composition in a weight ratio between 5:1 and 1:1.
B238. The composition of B230 or any preceding paragraph dependent thereon, wherein the gum (preferably xanthan gum) and glycerol are present within the composition in a weight ratio of about 3:1.
B239. The composition of B230 or any preceding paragraph dependent thereon, wherein the weight ratio of aloe vera to the combined weight of the gum (preferably xanthan gum) and glycerol is between 1:1 and 30:1.
B240. The composition of B230 or any preceding paragraph dependent thereon, wherein the weight ratio of aloe vera to the combined weight of the gum (preferably xanthan gum) and glycerol is between 5:1 and 20:1.
B241. The composition of B228-B240, wherein the composition comprises 0.1-70 wt % aloe vera.
B242. The composition of B228-B240, wherein the composition comprises 1-60 wt % aloe vera.
B243. The composition of B228-B240, wherein the composition comprises 2-50 wt % aloe vera.
B244. The composition of B228-B240, wherein the composition comprises 20-80 wt % aloe vera.
B245. The composition of B228-B240, wherein the composition comprises 30-70 wt % aloe vera.
B246. The composition of B228-B240, wherein the composition comprises 40-60 wt % aloe vera.

### Absent/Low-Level Ingredients

B247. The composition of B1-B227, wherein the composition is (substantially, preferably entirely) free of polymers.
B248. The composition of B1-B227, wherein the composition is (substantially, preferably entirely) free of polyvinyl alcohol (PVA).
B249. The composition of B1-B227, wherein the composition is (substantially, preferably entirely) free of polyalkylene glycol(s) (e.g. polyethylene glycol and/or polypropylene glycol).
B250. The composition of B1-B227, wherein the composition is (substantially, preferably entirely) free of polyalkylene glycol ester(s) (e.g. polyethylene glycol and/or polypropylene glycol ester(s)).
B251. The composition of B11-B227, wherein the composition is (substantially, preferably entirely) free of any compounds that comprise polyalkylene glycol ester moieties (e.g. polyethylene glycol and/or polypropylene glycol ester moieties).
B252. The composition of B11-B227, wherein the composition is (substantially, preferably entirely) free of polyethylene glycol.
B253. The composition of B11-B227, wherein the composition is (substantially, preferably entirely) free of polyethylene glycol ester(s).
B254. The composition of B1-B227, wherein the composition is (substantially, preferably entirely) free of any compounds that comprise polyethylene glycol ester moieties.
B255. The composition of B1-B254, wherein the composition is (substantially, preferably entirely) free of surfactants.
B256. The composition of B1-B254, wherein the composition is (substantially, preferably entirely) free of poloxamer surfactants.
B257. The composition of B11-B254, wherein the composition is (substantially, preferably entirely) free of poloxamer 407 (e.g. Pluronic F127).
B258. The composition of B1-B254, wherein the composition is (substantially, preferably entirely) free of polysorbate surfactants.
B259. The composition of B1-B254, wherein the composition is (substantially, preferably entirely) free of polysorbate 20.
B260. The composition of B1-B254, wherein the composition is (substantially, preferably entirely) free of PEG-40 castor oil (e.g. Killiphor RH 40, Cremophor RH40) surfactants.
B261. The composition of B1-B254, wherein the composition is (substantially, preferably entirely) free of poloxamer and polysorbate surfactants.
B262. The composition of B1-B254, wherein the composition is (substantially, preferably entirely) free of poloxamer. polysorbate, and PEG-40 castor oil surfactants.
B263. The composition of B1-B254, wherein the composition comprises at most 3 wt % surfactant(s), suitably at most 3 wt % of any surfactants defined in any of B255-B262.
B264. The composition of B1-B254, wherein the composition comprises at most 1 wt % surfactant(s), suitably at most 1 wt % of any surfactants defined in any of B255-B262.
B265. The composition of B1-B254, wherein the composition comprises at most 0.1 wt % surfactant(s), suitably at most 0.1 wt % of any surfactants defined in any of B255-B262.
B266. The composition of B1-B254, wherein the composition comprises at most 0.01 wt % surfactant(s), suitably at most 0.01 wt % of any surfactants defined in any of B255-B262.
B267. The composition of B1-B254, wherein the composition comprises at most 0.001 wt % surfactant(s), suitably at most 0.001 wt % of any surfactants defined in any of B255-B262.
B268. The composition of B11-B267, wherein the composition is (substantially, preferably entirely) free of lipids other than those stipulated.
B269. The composition of B1-B268, wherein the composition is (substantially, preferably entirely) free of solvents (i.e. compounds that are liquids at SATP) other than water.

### Structure, State, Properties of Curcumin/Glycerides

B270. The composition of B87-B144 or any preceding paragraph dependent thereon, insofar as it depends on B1-B69 (i.e. such that the composition comprises both a curcumin compound and glyceride(s)), wherein the curcumin compound (or relevant sub-definition(s) thereof) is solubilised and/or encapsulated within the glyceride(s) (or relevant sub-definition(s) thereof).
B271. The composition of B270, wherein the curcumin compound is dissolved within the glyceride(s).
B272. The composition of B270-B271, wherein the curcumin compound is encapsulated within the glyceride(s).
B273. The composition of B270-B272, wherein the curcumin compound is both dissolved within and encapsulated within the glyceride(s).
B274. The composition of B270-B273, wherein the composition comprises lamella structures comprising both the curcumin compound and the glyceride(s). Lamella structures are especially predominant in dehydrated (or low water content) systems.
B275. The composition of B274, wherein the composition is non-transparent (i.e. is opaque) at SATP. This is generally due to a prevailing non-isotropic structure (e.g. lamella structure).
B276. The composition of B274-B275, wherein the composition is non-transparent (i.e. is opaque) at temperatures within +/−5° C. (preferably +/−1° C.) of the melting point of the either the composition or of the predominant lipid or glyceride(s).
B277. The composition of B274-B276, wherein the composition becomes transparent upon melting (e.g. when heated at least 5° C. above the melting point of either the composition or of the predominant lipid or glyceride(s)).
B278. The composition of B274-B277, wherein the lamella structures are characterised by a liquid crystalline fluid lamellar phase comprising both the curcumin compound and the glyceride(s).
B279. The composition of B274-B278, wherein one or more entire molecules of the curcumin compound are contained within a single lamella that also contains the glyceride(s).
B280. The composition of B274-B279, wherein one or more molecules of the curcumin compound bridge between two separate lamellae which each contains the glyceride(s) (i.e. such molecules of the curcumin compound are only partially contained within a single lamella).
B281. The composition of B270-B272, wherein the composition comprises both the curcumin compound and the glyceride(s) in a cubic phase, preferably a bicontinuous cubic Pn3m phase. Such cubic phases are especially predominant in hydrated (water-containing, or high water content, especially >20 wt %) systems.
B282. The composition of B281, wherein the composition comprises some lamella structures comprising both the curcumin compound and the glyceride(s).
B283. The composition of B281-B282, wherein the composition is non-transparent at SATP. This is generally due to a prevailing non-isotropic structure of the lamella structures.
B284. The composition of B281-B283, wherein the composition is or becomes transparent at 40° C. or higher, suitably at 30° C. or higher. This is suitably due to lamella structures being converted back into a bicontinuous cubic Pn3m phase, which is substantially isotropic. This transparent behaviour generally holds until very high curcumin contents are reached (e.g. at or above 200 mg/g curcumin compound, or at or above 200:1000 weight ratio of curcumin compound to glyceride(s)), at which point the compositions become more opaque.

### Concentration Curcumin Compound

B285. The composition of B1-B69 or any preceding paragraph dependent thereon, wherein the composition comprises 0.001-50 wt % curcumin compound (preferably curcumin).
B286. The composition of B1-B69 or any preceding paragraph dependent thereon, wherein the composition comprises 0.01-40 wt % curcumin compound (preferably curcumin).
B287. The composition of B1-B69 or any preceding paragraph dependent thereon, wherein the composition comprises 0.1-30 wt % curcumin compound (preferably curcumin).
B288. The composition of B1-B69 or any preceding paragraph dependent thereon, wherein the composition comprises 0.01-10 wt % curcumin compound (preferably curcumin).
B289. The composition of B1-B69 or any preceding paragraph dependent thereon, wherein the composition comprises 0.001-3 wt % curcumin compound (preferably curcumin).
B290. The composition of B1-B69 or any preceding paragraph dependent thereon, wherein the composition comprises 0.01-1 wt % curcumin compound (preferably curcumin).
B291. The composition of B1-B69 or any preceding paragraph dependent thereon, wherein the composition comprises 0.1-4 wt % curcumin compound (preferably curcumin).
B292. The composition of B1-B69 or any preceding paragraph dependent thereon, wherein the composition comprises 0.1-5 wt % curcumin compound (preferably curcumin).
B293. The composition of B1-B69 or any preceding paragraph dependent thereon, wherein the composition comprises 0.001-2 wt % curcumin compound (preferably curcumin).
B294. The composition of B1-B69 or any preceding paragraph dependent thereon, wherein the composition comprises 0.005-1 wt % curcumin compound (preferably curcumin).
B295. The composition of B1-B69 or any preceding paragraph dependent thereon, wherein the composition comprises 0.01-0.1 wt % curcumin compound (preferably curcumin).
B296. The composition of B1-B69 or any preceding paragraph dependent thereon, wherein the composition comprises 15-40 wt % curcumin compound (preferably curcumin).
B297. The composition of B1-B69 or any preceding paragraph dependent thereon, wherein the composition comprises 0.1-1 wt % curcumin compound (preferably curcumin).
B298. The composition of B1-B69 or any preceding paragraph dependent thereon, wherein the composition comprises 0.5-1.5 wt % curcumin compound (preferably curcumin).
B299. The composition of B1-B69 or any preceding paragraph dependent thereon, wherein the composition comprises 1-10 wt % curcumin compound (preferably curcumin).
B300. The composition of B1-B69 or any preceding paragraph dependent thereon, wherein the composition comprises 5-10 wt % curcumin compound (preferably curcumin).
B301. The composition of B1-B69 or any preceding paragraph dependent thereon, wherein the composition comprises 0.1-3 wt % curcumin compound (preferably curcumin).
B302. The composition of B1-B69 or any preceding paragraph dependent thereon, wherein the composition comprises 0.5-1.5 wt % curcumin compound (preferably curcumin).
B303. The composition of B1-B69 or any preceding paragraph dependent thereon, wherein the composition comprises 0.5-5 wt % curcumin compound (preferably curcumin).
B304. The composition of B1-B69 or any preceding paragraph dependent thereon, wherein the composition comprises 1-2 wt % curcumin compound (preferably curcumin).
B305. The composition of B1-B69 or any preceding paragraph dependent thereon, wherein the composition comprises 10-30 wt % curcumin compound (preferably curcumin).
B306. The composition of B1-B69 or any preceding paragraph dependent thereon, wherein the composition comprises 17.5-22.5 wt % curcumin compound (preferably curcumin).
B307. The composition of B1-B69 or any preceding paragraph dependent thereon, wherein the composition comprises 0.005-0.5 wt % curcumin compound (preferably curcumin).
B308. The composition of B1-B69 or any preceding paragraph dependent thereon, wherein the composition comprises 0.01-0.1 wt % curcumin compound (preferably curcumin).
B309. The composition of B1-B69 or any preceding paragraph dependent thereon, wherein the composition comprises 0.1-3 wt % curcumin compound (preferably curcumin).
B310. The composition of B1-B69 or any preceding paragraph dependent thereon, wherein the composition comprises 0.5-1.7 wt % curcumin compound (preferably curcumin).
B311. The composition of B1-B69 or any preceding paragraph dependent thereon, wherein the curcumin compound (preferably curcumin) is present within the composition at a concentration between 0.5 and 800 mg/g (i.e. 0.05-80 wt % of the total weight of the composition).
B312. The composition of B1-B69 or any preceding paragraph dependent thereon, wherein the curcumin compound (preferably curcumin) is present within the composition at a concentration between 10 and 500 mg/g.
B313. The composition of B1-B69 or any preceding paragraph dependent thereon, wherein the curcumin compound (preferably curcumin) is present within the composition at a concentration greater than equal to 3.5 mg/g (i.e. 20.35 wt % of the total weight of the composition).
B314. The composition of B1-B69 or any preceding paragraph dependent thereon, wherein the curcumin compound (preferably curcumin) is present within the composition at a concentration greater than equal to 0.1 mg/g.
B315. The composition of B1-B69 or any preceding paragraph dependent thereon, wherein the curcumin compound (preferably curcumin) is present within the composition at a concentration greater than equal to 1 mg/g.
B316. The composition of B1-B69 or any preceding paragraph dependent thereon, wherein the curcumin compound (preferably curcumin) is present within the composition at a concentration greater than equal to 5 mg/g.
B317. The composition of B1-B69 or any preceding paragraph dependent thereon, wherein the curcumin compound (preferably curcumin) is present within the composition at a concentration greater than equal to 10 mg/g.
B318. The composition of B1-B69 or any preceding paragraph dependent thereon, wherein the curcumin compound (preferably curcumin) is present within the composition at a concentration greater than equal to 15 mg/g.
B319. The composition of B1-B69 or any preceding paragraph dependent thereon, wherein the curcumin compound (preferably curcumin) is present within the composition at a concentration greater than equal to 30 mg/g.
B320. The composition of B1-B69 or any preceding paragraph dependent thereon, wherein the curcumin compound (preferably curcumin) is present within the composition at a concentration greater than equal to 60 mg/g.
B321. The composition of B1-B69 or any preceding paragraph dependent thereon, wherein the curcumin compound (preferably curcumin) is present within the composition at a concentration greater than equal to 70 mg/g.
B322. The composition of B1-B69 or any preceding paragraph dependent thereon, wherein the curcumin compound (preferably curcumin) is present within the composition at a concentration greater than equal to 100 mg/g.
B323. The composition of B1-B69 or any preceding paragraph dependent thereon, wherein the curcumin compound (preferably curcumin) is present within the composition at a concentration greater than equal to 300 mg/g.
B324. The composition of B1-B69 or any preceding paragraph dependent thereon, wherein the curcumin compound (preferably curcumin) is present within the composition at a concentration less than equal to 600 mg/g.
B325. The composition of B1-B69 or any preceding paragraph dependent thereon, wherein the curcumin compound (preferably curcumin) is present within the composition at a concentration less than equal to 500 mg/g.
B326. The composition of B1-B69 or any preceding paragraph dependent thereon, wherein the curcumin compound (preferably curcumin) is present within the composition at a concentration less than equal to 200 mg/g.
B327. The composition of B1-B69 or any preceding paragraph dependent thereon, wherein the curcumin compound (preferably curcumin) is present within the composition at a concentration less than equal to 100 mg/g.
B328. The composition of B1-B69 or any preceding paragraph dependent thereon, wherein the curcumin compound (preferably curcumin) is present within the composition at a concentration between 2-8 mg/g.
B329. The composition of B1-B69 or any preceding paragraph dependent thereon, wherein the curcumin compound (preferably curcumin) is present within the composition at a concentration between 3-7 mg/g.
B330. The composition of B1-B69 or any preceding paragraph dependent thereon, wherein the curcumin compound (preferably curcumin) is present within the composition at a concentration between 4-6 mg/g.
B331. The composition of B1-B69 or any preceding paragraph dependent thereon, wherein the curcumin compound (preferably curcumin) is present within the composition at a concentration between 5-10 mg/g.
B332. The composition of B1-B69 or any preceding paragraph dependent thereon, wherein the curcumin compound (preferably curcumin) is present within the composition at a concentration between 6.5-8.5 mg/g.
B333. The composition of B1-B69 or any preceding paragraph dependent thereon, wherein the curcumin compound (preferably curcumin) is present within the composition at a concentration between 5-30 mg/g.
B334. The composition of B1-B69 or any preceding paragraph dependent thereon, wherein the curcumin compound (preferably curcumin) is present within the composition at a concentration between 10-20 mg/g.
B335. The composition of B1-B69 or any preceding paragraph dependent thereon, wherein the curcumin compound (preferably curcumin) is present within the composition at a concentration between 10-500 mg/g.
B336. The composition of B1-B69 or any preceding paragraph dependent thereon, wherein the curcumin compound (preferably curcumin) is present within the composition at a concentration between 11-19 mg/g.
B337. The composition of B1-B69 or any preceding paragraph dependent thereon, wherein the curcumin compound (preferably curcumin) is present within the composition at a concentration between 10-40 mg/g.
B338. The composition of B1-B69 or any preceding paragraph dependent thereon, wherein the curcumin compound (preferably curcumin) is present within the composition at a concentration between 15-35 mg/g.
B339. The composition of B1-B69 or any preceding paragraph dependent thereon, wherein the curcumin compound (preferably curcumin) is present within the composition at a concentration between 20-30 mg/g.
B340. The composition of B1-B69 or any preceding paragraph dependent thereon, wherein the curcumin compound (preferably curcumin) is present within the composition at a concentration between 30-500 mg/g.
B341. The composition of B1-B69 or any preceding paragraph dependent thereon, wherein the curcumin compound (preferably curcumin) is present within the composition at a concentration between 70-700 mg/g.
B342. The composition of B1-B69 or any preceding paragraph dependent thereon, wherein the curcumin compound (preferably curcumin) is present within the composition at a concentration between 60-500 mg/g.
B343. The composition of B1-B69 or any preceding paragraph dependent thereon, wherein the curcumin compound (preferably curcumin) is present within the composition at a concentration between 40-150 mg/g (i.e. 6-10 wt %).
B344. The composition of B1-B69 or any preceding paragraph dependent thereon, wherein the curcumin compound (preferably curcumin) is present within the composition at a concentration between 60-100 mg/g (i.e. 6-10 wt %).
B345. The composition of B1-B69 or any preceding paragraph dependent thereon, wherein the curcumin compound (preferably curcumin) is present within the composition at a concentration between 65-95 mg/g.
B346. The composition of B1-B69 or any preceding paragraph dependent thereon, wherein the curcumin compound (preferably curcumin) is present within the composition at a concentration between 100-700 mg/g.
B347. The composition of B1-B69 or any preceding paragraph dependent thereon, wherein the curcumin compound (preferably curcumin) is present within the composition at a concentration between 200-600 mg/g.
B348. The composition of B1-B69 or any preceding paragraph dependent thereon, wherein the curcumin compound (preferably curcumin) is present within the composition at a concentration between 300-500 mg/g.
B349. The composition of B1-B69 or any preceding paragraph dependent thereon, wherein the curcumin compound (preferably curcumin) is present within the composition at a concentration between 350-450 mg/g.

### Ratio Curcumin/Glycerides

B350. The composition of B87-B144 or any preceding paragraph dependent thereon, insofar as it depends on B1-B69 (i.e. such that the composition comprises both a curcumin compound and glyceride(s)—i.e. stipulated glycerides), wherein the curcumin compound (or relevant sub-definition(s) thereof) and the glyceride(s) (or relevant sub-definition(s) thereof) are present within the composition at a defined relative weight ratio.
B351. The composition of B350, wherein the weight ratio of the curcumin compound to the glyceride(s) is between 0.5:1000 and 800:1000 (i.e. 5:10000-8:10).
B352. The composition of B350, wherein the weight ratio of the curcumin compound to the glyceride(s) is between 10:1000 and 500:1000.
B353. The composition of B350, wherein the weight ratio of the curcumin compound to the glyceride(s) is between 30:1000 and 500:1000.
B354. The composition of B350, wherein the weight ratio of the curcumin compound to the glyceride(s) is between 70:1000 and 700:1000.
B355. The composition of B350, wherein the weight ratio of the curcumin compound to the glyceride(s) is between 60:1000 and 500:1000.
B356. The composition of B350, wherein the weight ratio of the curcumin compound to the glyceride(s) is between 0.5:1000 and 300:1000.
B357. The composition of B350, wherein the weight ratio of the curcumin compound to the glyceride(s) is between 0.5:1000 and 30:1000.
B358. The composition of B350, wherein the weight ratio of the curcumin compound to the glyceride(s) is between 0.5:1000 and 20:1000.
B359. The composition of B350, wherein the weight ratio of the curcumin compound to the glyceride(s) is between 5:1000 and 100:1000.
B360. The composition of B350, wherein the weight ratio of the curcumin compound to the glyceride(s) is between 2:1000 and 8:1000.
B361. The composition of B350, wherein the weight ratio of the curcumin compound to the glyceride(s) is between 4:1000 and 6:1000.
B362. The composition of B350, wherein the weight ratio of the curcumin compound to the glyceride(s) is between 5:1000 and 10:1000.
B363. The composition of B350, wherein the weight ratio of the curcumin compound to the glyceride(s) is between 6.5:1000 and 8.5:1000.
B364. The composition of B350, wherein the weight ratio of the curcumin compound to the glyceride(s) is between 5:1000 and 30:1000.
B365. The composition of B350, wherein the weight ratio of the curcumin compound to the glyceride(s) is between 10:1000 and 20:1000.
B366. The composition of B350, wherein the weight ratio of the curcumin compound to the glyceride(s) is between 11:1000 and 19:1000.
B367. The composition of B350, wherein the weight ratio of the curcumin compound to the glyceride(s) is between 10:1000 and 40:1000.
B368. The composition of B350, wherein the weight ratio of the curcumin compound to the glyceride(s) is between 15:1000 and 35:1000.
B369. The composition of B350, wherein the weight ratio of the curcumin compound to the glyceride(s) is between 20:1000 and 30:1000.
B370. The composition of B350, wherein the weight ratio of the curcumin compound to the glyceride(s) is between 40:1000 and 150:1000.
B371. The composition of B350, wherein the weight ratio of the curcumin compound to the glyceride(s) is between 60:1000 and 100:1000.
B372. The composition of B350, wherein the weight ratio of the curcumin compound to the glyceride(s) is between 65:1000 and 95:1000.
B373. The composition of B350, wherein the weight ratio of the curcumin compound to the glyceride(s) is between 100:1000 and 700:1000.
B374. The composition of B350, wherein the weight ratio of the curcumin compound to the glyceride(s) is between 200:1000 and 600:1000.
B375. The composition of B350, wherein the weight ratio of the curcumin compound to the glyceride(s) is between 300:1000 and 500:1000.
B376. The composition of B350, wherein the weight ratio of the curcumin compound to the glyceride(s) is between 350:1000 and 450:1000.
   Concentration of other glyceride(s)/Lipids
B377. The composition of B87-B144 or any preceding paragraph dependent thereon, insofar as it depends on B1-B69 (i.e. such that the composition comprises both a curcumin compound and glyceride(s)), wherein the stipulated (i.e. the defined) glyceride(s) (or relevant sub-definition(s) thereof) are the predominant glyceride(s), and suitably the predominant lipid(s) (e.g. fatty-acid-containing compounds), within the composition. Suitably, all glyceride(s) (including predominant glyceride(s)) and lipids within the composition are collectively termed lipid compounds.
B378. The composition of B87-B144 or any preceding paragraph dependent thereon, insofar as it depends on B1-B69 (i.e. such that the composition comprises both a curcumin compound and glyceride(s)), wherein the stipulated (i.e. the defined) glyceride(s) (or relevant sub-definition(s) thereof) constitute at least 50 wt % of the total amount of glyceride(s) present within the composition (i.e. the “50 wt %” is relative to total glyceride(s) rather than the composition as a whole), and preferably constitute at least 50 wt % of the total amount of lipid(s) present within the composition (i.e. the “50 wt %” is relative to total lipid(s) rather than the composition as a whole). All glyceride(s) (including predominant glyceride(s)) and other lipids within the composition are collectively termed lipid compounds or just “lipids”.
B379. The composition of B378, wherein the stipulated (or defined) glyceride(s) constitute at least 70 wt % of the total amount of glyceride(s) present within the composition.
B380. The composition of B378, wherein the stipulated (or defined) glyceride(s) constitute at least 85 wt % of the total amount of glyceride(s) present within the composition.
B381. The composition of B378, wherein the stipulated (or defined) glyceride(s) constitute at least 89 wt % of the total amount of glyceride(s) present within the composition.
B382. The composition of B378, wherein the stipulated (or defined) glyceride(s) constitute at least 95 wt % of the total amount of glyceride(s) present within the composition.
B383. The composition of B378, wherein the stipulated (or defined) glyceride(s) constitute at least 70 wt % of the total amount of lipids present within the composition.
B384. The composition of B378, wherein the stipulated (or defined) glyceride(s) constitute at least 85 wt % of the total amount of lipids present within the composition.
B385. The composition of B378, wherein the stipulated (or defined) glyceride(s) constitute at least 89 wt % of the total amount of lipids present within the composition.
B386. The composition of B378, wherein the stipulated (or defined) glyceride(s) constitute at least 95 wt % of the total amount of lipids present within the composition.
B387. The composition of B1-B386, wherein glyceride(s) (stipulated and/or unstipulated) constitute at least 70 wt % of the total amount of lipids present within the composition.
B388. The composition of B387, wherein glyceride(s) (stipulated and/or unstipulated) constitute at least 85 wt % of the total amount of lipids present within the composition.
B389. The composition of B388, wherein glyceride(s) (stipulated and/or unstipulated) constitute at least 95 wt % of the total amount of lipids present within the composition.
B390. The composition of B389, wherein glyceride(s) (stipulated and/or unstipulated) constitute at least 99 wt % of the total amount of lipids present within the composition.

### Concentration of Water

B391. The composition of B145 or any preceding paragraph dependent thereon (particularly those that contain both curcumin compound and glyceride(s)), wherein the composition comprises 0-99 wt % water.
B392. The composition of B391, wherein the composition comprises 0.01-95 wt % water.
B393. The composition of B391, wherein the composition comprises 1-97 wt % water.
B394. The composition of B391, wherein the composition comprises less than or equal to 80 wt % water (i.e. the composition has at most 80 wt % water, and may contain 0 wt % water).
B395. The composition of B391, wherein the composition comprises less than or equal to 60 wt % water (i.e. the composition has at most 60 wt % water, and may contain 0 wt % water).
B396. The composition of B391, wherein the composition comprises less than or equal to 40 wt % water (i.e. the composition has at most 40 wt % water, and may contain 0 wt % water).
B397. The composition of B391, wherein the composition comprises less than or equal to 5 wt % water (i.e. the composition has at most 5 wt % water, and may contain 0 wt % water).
B398. The composition of B391, wherein the composition comprises less than or equal to 2 wt % water.
B399. The composition of B391, wherein the composition comprises less than or equal to 1 wt % water.
B400. The composition of B391, wherein the composition comprises 10-60 wt % water.
B401. The composition of B391, wherein the composition comprises 15-60 wt % water.
B402. The composition of B391, wherein the composition comprises 20-45 wt % water.
B403. The composition of B391, wherein the composition comprises 25-43 wt % water.
B404. The composition of B391, wherein the composition comprises 32-41 wt % water.
B405. The composition of B400-B404, wherein the composition is formed by adding a required amount of water to a composition of B394-B399, and suitably thereafter mixing, suitably with sufficient heating to melt the composition of B394-B399 or at least the glyceride(s) thereof. Wherever curcumin compound is to be added (e.g. to form a hydrated composition containing significant quantities of curcumin), suitably curcumin addition is performed by mixing additional curcumin with the composition of B394-B399 (preferably heated so as to be in a melted state) before the addition of water. Products are suitably stored at or below 20° C.
B406. The composition of B391, wherein the composition comprises 40-85 wt % water. Such compositions may suitable include a surfactant.
B407. The composition of B391, wherein the composition comprises 75-85 wt % water. Such compositions may suitable include a surfactant.
B408. The composition of B391, wherein the composition comprises 85-99.9 wt % water. Such compositions may suitable include a surfactant.
B409. The composition of B391, wherein the composition comprises 45-99 wt % water. Such compositions may suitable include a surfactant.
B410. The composition of B391, wherein the composition comprises 70-98 wt % water. Such compositions may suitable include a surfactant.
B411. The composition of B391, wherein the composition comprises 90-97 wt % water. Such compositions may suitable include a surfactant.
B412. The composition of B406-B411, wherein the composition is formed by adding a required amount of water to a composition of B394-B405, and suitably thereafter mixing, suitably with sufficient heating to melt the composition of B394-B405 or at least the glyceride(s) thereof.

### Additional Composition-Specific Facets

B413. The composition of B1-B412, wherein at least 1 wt % of the composition is constituted by the stipulated ingredients.
B414. The composition of B1-B412, wherein at least 10 wt % of the composition is constituted by the stipulated ingredients.
B415. The composition of B1-B412, wherein at least 15 wt % of the composition is constituted by the stipulated ingredients.
B416. The composition of B1-B412, wherein at least 20 wt % of the composition is constituted by the stipulated ingredients.
B417. The composition of B1-B412, wherein at least 50 wt % of the composition is constituted by the stipulated ingredients.
B418. The composition of B1-B412, wherein at least 70 wt % of the composition is constituted by the stipulated ingredients.
B419. The composition of B1-B412, wherein at least 80 wt % of the composition is constituted by the stipulated ingredients.
B420. The composition of B1-B412, wherein at least 85 wt % of the composition is constituted by the stipulated ingredients.
B421. The composition of B1-B412, wherein at least 90 wt % of the composition is constituted by the stipulated ingredients.
B422. The composition of B1-B412, wherein at least 95 wt % of the composition is constituted by the stipulated ingredients.
B423. The composition of B1-B412, wherein at least 99 wt % of the composition is constituted by the stipulated ingredients.
B424. The composition of B1-B412, wherein the composition consists of the stipulated ingredients.
B425. The composition of B1-B412, wherein at least 1 wt % of the composition is constituted by the stipulated curcumin compound and stipulated glyceride(s) (e.g. as per the glyceride(s) stipulated in B87-B144).
B426. The composition of B1-B412, wherein at least 10 wt % of the composition is constituted by the stipulated curcumin compound and stipulated glyceride(s) (e.g. as per the glyceride(s) stipulated in B87-B144).
B427. The composition of B1-B412, wherein at least 15 wt % of the composition is constituted by the stipulated curcumin compound and stipulated glyceride(s) (e.g. as per the glyceride(s) stipulated in B87-B144).
B428. The composition of B1-B412, wherein at least 20 wt % of the composition is constituted by the stipulated curcumin compound and stipulated glyceride(s) (e.g. as per the glyceride(s) stipulated in B87-B144).
B429. The composition of B1-B412, wherein at least 50 wt % of the composition is constituted by the stipulated curcumin compound and stipulated glyceride(s) (e.g. as per the glyceride(s) stipulated in B87-B144).
B430. The composition of B1-B412, wherein at least 70 wt % of the composition is constituted by the stipulated curcumin compound and stipulated glyceride(s) (e.g. as per the glyceride(s) stipulated in B87-B144).
B431. The composition of B1-B412, wherein at least 80 wt % of the composition is constituted by the stipulated curcumin compound and stipulated glyceride(s) (e.g. as per the glyceride(s) stipulated in B87-B144).
B432. The composition of B1-B412, wherein at least 85 wt % of the composition is constituted by the stipulated curcumin compound and stipulated glyceride(s) (e.g. as per the glyceride(s) stipulated in B87-B144).
B433. The composition of B1-B412, wherein at least 90 wt % of the composition is constituted by the stipulated curcumin compound and stipulated glyceride(s) (e.g. as per the glyceride(s) stipulated in B87-B144).
B434. The composition of B1-B412, wherein at least 95 wt % of the composition is constituted by the stipulated curcumin compound and stipulated glyceride(s) (e.g. as per the glyceride(s) stipulated in B87-B144).
B435. The composition of B1-B412, wherein at least 99 wt % of the composition is constituted by the stipulated curcumin compound and stipulated glyceride(s) (e.g. as per the glyceride(s) stipulated in B87-B144).
B436. The composition of B1-B435, wherein centrifugation (e.g. 3000 rpm for 5 min at RT) of the composition yields less than 10 wt % sedimentation (i.e. less than 10 wt % of the composition as a whole).
B437. The composition of B1-B435, wherein centrifugation (e.g. 3000 rpm for 5 min at RT) of the composition yields less than 5 wt % sedimentation (i.e. less than 5 wt % of the composition as a whole).
B438. The composition of B1-B435, wherein centrifugation (e.g. 3000 rpm for 5 min at RT) of the composition yields less than 2 wt % sedimentation (i.e. less than 2 wt % of the composition as a whole).
B439. The composition of B1-B435, wherein centrifugation (e.g. 3000 rpm for 5 min at RT) of the composition yields less than 1 wt % sedimentation (i.e. less than 1 wt % of the composition as a whole).
B440. The composition of B1-B435, wherein centrifugation (e.g. 3000 rpm for 5 min at RT) of the composition yields less than 0.1 wt % sedimentation (i.e. less than 0.1 wt % of the composition as a whole).
B441. The composition of B1-B435, wherein centrifugation (e.g. 3000 rpm for 5 min at RT) of the composition yields less than 0.01 wt % sedimentation (i.e. less than 0.01 wt % of the composition as a whole).
B442. The composition of B1-B435, wherein centrifugation (e.g. 3000 rpm for 5 min at RT) of the composition yields (substantially) no sedimentation.
B443. The composition of B1-B435, wherein centrifugation (e.g. 3000 rpm for 5 min at RT) of the composition yields either no sedimentation or sedimentation containing less than 50 wt % of the curcumin compound present within the composition (i.e. this refers to 50 wt % of the total amount of curcumin compound, not 50 wt % of the composition as a whole).
B444. The composition of B443, wherein centrifugation (e.g. 3000 rpm for 5 min at RT) of the composition yields either no sedimentation or sedimentation containing less than 20 wt % of the curcumin compound present within the composition.
B445. The composition of B443, wherein centrifugation (e.g. 3000 rpm for 5 min at RT) of the composition yields either no sedimentation or sedimentation containing less than 10 wt % of the curcumin compound present within the composition.
B446. The composition of B443, wherein centrifugation (e.g. 3000 rpm for 5 min at RT) of the composition yields either no sedimentation or sedimentation containing less than 5 wt % of the curcumin compound present within the composition.
B447. The composition of B443, wherein centrifugation (e.g. 3000 rpm for 5 min at RT) of the composition yields either no sedimentation or sedimentation containing less than 1 wt % of the curcumin compound present within the composition.
B448. The composition of B443, wherein centrifugation (e.g. 3000 rpm for 5 min at RT) of the composition yields either no sedimentation or sedimentation containing less than 0.1 wt % of the curcumin compound present within the composition.
B449. The composition of B443, wherein centrifugation (e.g. 3000 rpm for 5 min at RT) of the composition yields either no sedimentation of any curcumin compound present within the composition.
B450. The composition of B1-B449, wherein the composition is a solid, fluid, viscoelastic or semi-solid.

The definitions set forth in any of paragraphs B1-B450 may be applied (especially where compatible within the given context) to any of the specific embodiments set forth in A1-A131 or indeed any other definitions, embodiments, or aspects set forth herein in relation to the composition.

### EXAMPLES

### Materials and Equipment

The following materials and equipment were used:
Curcumin (Cambridge Biosciences, Cambridge, UK), monoolein (MO) (Croda Europe Ltd.), monolinolein (ML) (Sigma-Aldrich, UK), Dimodan-U (DU) (mixture of MO and ML) (Danisco (UK) Limited), butter oil (BO) (prepared from “unsalted English Butter” by Sainsbury's, UK after following the protocol described in Kulkarni, C.V. Ultrasonic processing of butter oil (ghee) into oil-in-water emulsions J. Food Process Preserv. 2017; 41:e13170).

### Analytical Protocols

The following analytical protocols were used: Nuclear Magnetic Resonance (NMR) spectra were obtained using Bruker 300 MHz and 400 MHz NMR spectrometers. The ultraviolet visible (UV) spectra were obtained using UV-1600PC, VWR, UK with quartz cells. The SAXSpace instrument (Anton Paar, Graz, Austria) was utilized for Small angle X-ray scattering (SAXS) studies. DSC thermograms were obtains using Q2000 Differential Scanning Calorimeter (TA Instruments, USA). Sample bottle photographs were captured using Samsung A5 mobile phone camera. Sonics & Materials Vibra-Cell VCX750, Jencons, UK was utilized for ultrasonic preparation of emulsion samples.

### General Summary of Formulations Tested

TABLE 1
Formulations Tested

| | | | Maximum | | |
|---|---|---|---|---|---|
| | | | curcumin | | |
| | | | content | | |
| | Name of | Formulation | (mg/g of | | |
| No. | Formulation | composition | lipid) | Proportion (wt %) | Comment |
| 1 | F00-BO | BO (mixture | <0.5 | a = 0.05%, b = | very low solubility |
| | | of TGs) | | 99.95% | |
| 2 | F00-BO | BO + Water | <0.5 | a = 0.05%, b = | very low solubility |
| | | | | 98.95%, c < 1.0 | |
| 3 | F01-MO | MO | 5.0 ± 1.0 | a = 0.5%, b = 99.5% | good solubility |
| 4 | F02-MO | MO + Water | 15.0 ± 4.0 | a = 0.89%, b = | very good solubility |
| | | | | 59.5%, c = 39.6% | |
| 5 | F01-DU | DU | 7.5 ± 1.0 | a = 0.75%, b = | good solubility |
| | | | | 99.25% | |
| 6 | F01-DU-E | DU | 80.0 ± 15 | a = 8.0%, b = 92.0% | encapsulation (stays |
| | | | | | homogeneous in lipid |
| | | | | | system) |
| 7 | F02-DU | DU + Water | 25.0 ± 5 | a = 1.5%, b = 59.1%, | very good solubility |
| | | | | c = 39.4% | |
| 8 | F02-DU-E | DU + Water | 400.0 ± 40 | a = 19.4%, b = | encapsulation (stays |
| | | | | 48.4%, c = 32.2% | homogeneous in lipid |
| | | | | | system) |
| 9 | F03-DU-L | DU dispersions (<15% | 7.5 ± 1.5 | a = 0.04%, b = 4.9%, | curcumin is soluble and |
| | (low lipid | lipid phase) + | | c = 94.5%, d = 0.47% | particulate emulsion is |
| | phase conc.) | stabilizer in water | | | stable |
| 10 | F03-DU-H | DU dispersions (>15% | 80.0 ± 15 | a = 1.2, b ≥ 14.8, | encapsulation (stays |
| | (high lipid | lipid phase) + | | c ≤ 83.6, | homogeneous in lipid |
| | phase conc.) | water or stabilizer | | d ≥ 0.42 | system) |
| | | in water | | | |

a = curcumin,
b = lipid,
c = water,
d = surfactant stabilizer

The compositions of Table 1 were prepared as set forth hereinafter so as to afford the indicated wt % proportions of each respective ingredient, and to produce compositions containing the indicated weight ratio of mg of curcumin per g of lipid(s).

Formulations of the invention involve lipid-based formulations that are capable of solubilizing and/or encapsulating high concentrations of curcumin (or derivatives thereof). The formulations contain lipids, preferably one or more monoglycerides containing at least one unsaturated moiety (preferably C₈-C₂₀, preferably C₁₈, preferably oleate and/or linoleate). The formulations can have a water content in the range of 0 to 95% (w/w), sometimes higher water content for aqueous dispersions. The formulation include curcumin (or derivatives thereof) in solubilized and/or encapsulated form, generally in a concentration range of 0.5-800 mg/g of lipid. In general, such formulations do not require surfactant stabilization, though in some embodiments they may. The formulations may include an optional oil (e.g. food-based) or other component to fine-tune physical form/s of the formulation(s). The formulations generally contain only ingredients that are generally recognized as safe (GRAS). The degree of curcumin loading within formulations can be modulated by altering the formulation composition, particularly, by adding various concentrations of additives including one or more natural oils, other lipids, fatty acids, bile salts (and/or bile acid(s)), hydrophilic molecules, hydrophobic molecules, and amphiphilic molecules. The physical state of the formulations can be modified into solid, fluid, and viscoelastic or semi-solid. The formulations can be tuned into various applicable forms including gel, cream, paste, dry film, powder, tablets, capsules, jellies, pastilles, gums, lotions, oil-in-water emulsion, water-in-oil emulsion and drops. The formulation can be suitable for all modes of administration into the body, preferably topical and oral mode of delivery.

Three different sets of formulations based on monoglycerides and mixtures in non-aqueous (F01), liquid crystalline bulk (F02) and nanostructured dispersion forms (F03) were prepared to obtain highly enhanced solubility, encapsulation and bioavailability of curcumin for various medicinal applications. Entries in the “Name of Formulation” column of Table 1 are prefixed with the relevant Formulation Code. (F01, F02, F03) to indicate from which base formulation they are derived/varied.

To form the F00-BO formulations: Curcumin powder was mixed with molten BO and stirred; slight change is colour (pale yellow to a little dark yellow) elucidates very low (<0.05%) solubility of curcumin in BO. BO being >99% lipid mixture does not mix with water; thus, curcumin solubility did not change after addition of water.

### Example 1—Curcumin Formulation (F01): Non-Aqueous Lipid Systems

Monoolein (MO) or Dimodan-U (DU) were employed to prepare non-aqueous formulations (F01) to solubilize or encapsulate curcumin (Table 1). DU is a commercial monoglyceride (MG) mixture containing about 96% distilled MGs of which 62% is ML and 25% is MO; remaining 4% constitute free fatty acids and diglycerides.

### F01 Preparation

F01 formulations were prepared as follows:
Curcumin, in the powder form, was mixed with either MO (for F01-MO) or DU (for F01-DU) in a 20 ml scintillation vial. A lipid phase can be formed of only MO or DU or it may can contain an optional other component utilized improve curcumin loading and/or fine-tune a physical form of the final formulation.
Mixture was heated using block heater or similar equipment to melt the lipid (around 40-45° C.).
Spatula or similar tools were used to blend the constituents until homogeneous.
Mixtures were heated again, if required, followed by homogenization by spatula.
Final formulation was stored at room temperature (around 20° C.) or in fridge (around 4° C.).

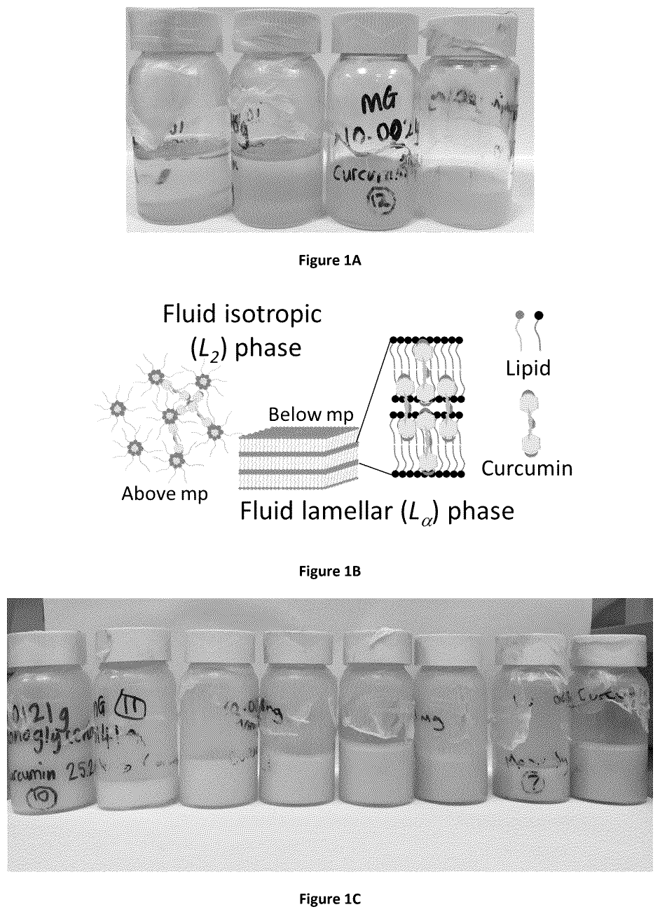

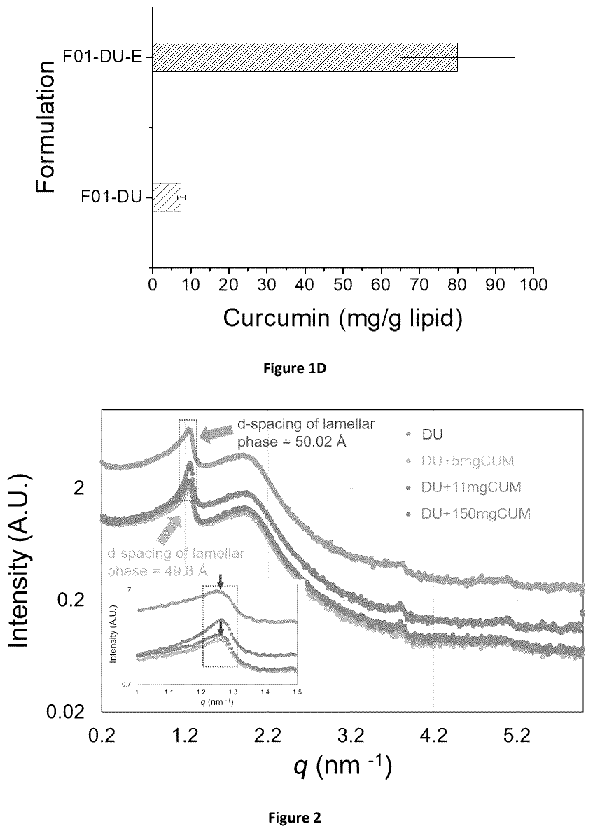

FIGS. 1A-D shows: A) various concentrations (1.2, 1.3, 1.5 and 2.0 mg/g, from left to right) of curcumin solubilized in molten DU; B) a schematic diagram depicting the likely solubilization/encapsulation of curcumin in DU, which forms liquid crystalline fluid lamellar (L_{α}) phase in dehydrated state, that melts into fluid isotropic (L₂) phase above 37° C.; C) Samples exhibiting encapsulation of curcumin (concentrations shown from L to R: 25, 30, 35, 40, 50, 60, 70 and 80 mg/g); and D) a histogram showing the curcumin concentrations in formulation F01 with error bars.

### Curcumin Solubility in MO and DU

The maximum solubility achieved in F01 for MO was 5.0±1.0 mg/g (Table 1), whereas it was 7.5±1.0 mg/g for DU (FIG. 1D). Solubilized curcumin remains in this form for several weeks at room temperature. Molten lipid is usually in inverse micellar (L₂) phase, which is isotropic and hence looks transparent (FIG. 1A). If cooled below 20±3° C., lipid crystallizes into crystalline lamellar phase (L_{c}), which has opaque solid appearance. The formulation regains its transparency (FIG. 1A) upon gentle heating above melting point (MP) of lipid. A very small amount of curcumin was found to sediment at the bottom of the vial when F01-DU was subjected to centrifugation at 3000 rpm for 5 min at room temperature. However, there was no any sedimentation after centrifugation for concentrations below 3.5±1.5 mg/g (Table 1).

### Curcumin Encapsulation

Further addition of curcumin above the maximum solubility point (7.5±1.0 mg/g for F01-DU), facilitates its encapsulation within the lipid system, which becomes opaque and remains like this even above the mp of DU. As high as 80±15 mg/g (FIGS. 1C and 1D) curcumin gets encapsulated in F01-DU without sedimentation at room temperature or at 40° C. (well above MP of DU).

### Proposed Mechanism of Solubilization and Encapsulation

Without wishing to be bound by theory, the inventor's insights, established during the course of their inventive endeavours, led to the following mechanism of solubilisation and encapsulation being proposed.

Monoglycerides (MG), namely monoolein (MO) and monolinolein (ML) are main constituents used in our formulations. These MGs, respectively, exhibit one and two ‘cis’ unsaturations, each producing ‘kink’ at the double bond position, leading to less tight molecular packing as compared to the TGs containing saturated chains. These packing constraints affect melting points, phase behaviour and fluidity attained by lipid systems—see Kulkarni, C. V.; Tang, T. Y.; Seddon, A. M.; Seddon, J. M.; Ces, O.; Templer, R. H., Engineering Bicontinuous Cubic Structures at the Nanoscale—the Role of Chain Splay. Soft Matter 2010, 6, 3191-3194. The optimum fluidity is advantageous for the diffusion and thus the solubility of curcumin in such systems. We have therefore, utilized MG systems to develop our first formulation (F01) as the formulations prepared from mixture of TGs (in the form of butter oil) showed very low (<0.5 mg/g) curcumin solubility (F00-BO in Table 1).

Not only physical spatial organization, but also amphiphilic nature of the host system aids towards the favourable interactions between curcumin and lipid, which might be supported by intermolecular hydrogen bonding (FIG. 1B). Inverse micelles (above mp) and multiple lamellar sheets (below mp) formed from pure lipids (FIG. 1B), in contrast to the single bilayer in liposome systems, practically allow high amount of curcumin to be loaded both within the bilayers and between them. The apparent viscosity of the lipid system also encourages the curcumin particles to remain homogeneously dispersed (and thus encapsulated) in the formulation well above the solubility limit.

### Further Characterisation

### Small Angle X-Ray Scattering (SAXS): Nanostructural Analysis

FIG. 2 shows identification of lamellar phase with and without curcumin using SAXS technique.

SAXS patterns for pure Dimodan U/J (DU) and DU with various concentrations of Curcumin (CUM) (FIG. 2). Pure DU forms lamellar phase¹ whose d-spacing (lattice parameter) was found to be 50.02 A—for more background see Mezzenga, R.; Meyer, C.; Servais, C.; Romoscanu, A. I.; Sagalowicz, L.; Hayward, R. C., Shear Rheology of Lyotropic Liquid Crystals: A Case Study. Langmuir 2005, 21 (8), 3322. Upon addition of curcumin the d-spacing of lamellar phase shows slight decrease. The bilayer thinning effect of curcumin was also observed earlier—for more background see Ingolfsson, H. I.; Koeppe, R. E.; Andersen, O. S., Curcumin is a Modulator of Bilayer Material Properties. Biochemistry 2007, 46 (36), 10384-10391.

### Differential Scanning Calorimetry (DSC): Thermal Analysis

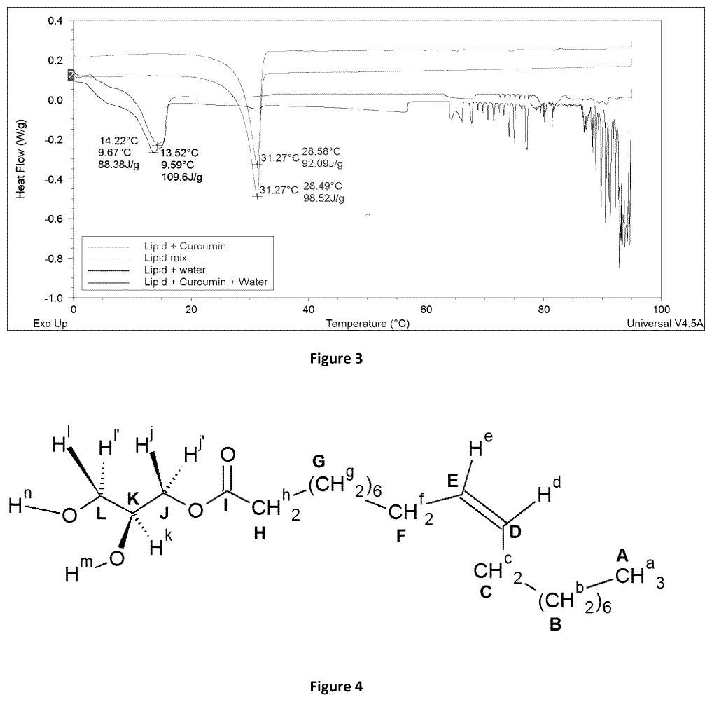

FIG. 3 shows DSC curves in heating-direction—showing curcumin effect in dry and wet states.

DSC thermogram (FIG. 3) depicts various curves in heating direction. The melting transition of lipid, both in dry (around 31° C.) and hydrated (around 13° C.) states, are shifted to higher temperatures. The incorporation of curcumin in lipid systems is confirmed from these transitions.

### Nuclear Magnetic Resonance (NMR) Spectroscopy: Retention of Curcumin Structure in Lipid Systems

NMR studies were performed on pure monoolein, a major component of DU with and without curcumin. The following observations confirm that the curcumin is retained in its native form even after solubilization in lipid systems.

In order to confirm that curcumin dissolves in molten monoolein and is present in the chemically unchanged form, NMR studies were carried out. In order to exclude the potential influence of any other compounds on solubility of curcumin in monoolein, spectra for neat monoolein were recorded in melt at 50° C., which is the optimum minimum temperature at which monoolein fluidises enough to give clear signals in NMR. Flame-sealed capillary with d₆-dmso was added for lock. The following spectra were recorded: ¹H, ¹³C{¹H}, ¹³C DEPT-135, ¹H-¹H COSY, ¹H-¹H NOESY, ¹H-¹H ROESY, ¹H-¹H TOCSY, ¹H-¹³C HSQC and ¹H-¹³C HMBC. These were used to assign resonances in ¹H and ¹³C NMR spectra, for the numbering scheme please refer to FIG. 4. Results are presented in Table 2 in tabulated form and graphically in FIG. 5 and FIG. 6.

TABLE 2
Tabulated ¹H and ¹³C NMR data for curcumin (refer to FIG. 4 for numbering scheme)

| ¹H NMR (neat melt at 50° C., flame sealed d₆-dmso capillary | ¹³C{¹H} NMR (neat melt at 50° C., flame sealed d₆-dmso |
|---|---|
| used for lock, 400 MHz): | capillary used for lock, 100 MHz): |
| 5.05-4.90 (m, 2H, H^{d} + H^{e}), 4.19 (br s, 1H, H^{m/n}), 3.99 (br s, 1H, | 172.42 (C^{I}, C = O), 128.57 (C^{E/D}), 128.50 (C^{D/E}), 69.12 |
| H^{n/m}), 3.77-3.71 (m, 2H, H^{j} + H^{j′}), 3.56-3.48 (m, 1H, H^{k}), 3.28 | (C^{K}), 64.02 (C^{J}), 62.31 (C^{L}), 32.85 (C^{H}), 30.88 + 28.77 + |
| (dd, 1H, H^{l}′, ²J_{l-l′} = 11.45 Hz, ³J_{l′-k} = 3.96 Hz), 3.19 (dd, 1H, H^{l}, | 28.74 + 28.73 + 28.51 + 28.30 + 28.28 + 28.26 + 28.20 + |
| ²J_{l-l′} = 11.45 Hz, ³Jₗ₋ₖ = 6.25 Hz), 1.96 (t, 2H, H^{h}, 7.61 Hz), 1.75- | 28.18 (C^{B+G}), 26.16 (C^{C/F}), 26.14 (C^{F/C}), 23.82 + 21.58 |
| 1.59 (m, 4H, H^{c} + H^{f}), 1.31-1.20 (m, 2H, H^{b} + H^{g}), 1.06-0.86 (m, | (C^{B+G}), 12.83 (C^{A}). |
| 22H, H^{b} + H^{g}), 0.54 (t, 3H, H^{a}, 6.99 Hz). | |

Following the determination of the solubility limit of curcumin in monoolein at 50° C., ¹H NMR spectrum of the solution of 5 mg curcumin in 1 g of monoolein was recorded. FIG. 7 shows the molecular structure of curcumin with the numbering scheme and the overlay of the ¹H NMR spectrum of neat monoolein (green-blue top trace) and the ¹H NMR spectrum of the saturated solution of curcumin in monoolein. The signals for curcumin were assigned based on typical chemical shifts and coupling constants, and are shown in Table 3 (OH and OCH₃ protons could not be detected due to signal overlaps/signal broadening); this assignment generally agrees with the literature data, however these are not directly comparable due to different solvents used (A. J. Yoon, H. Wu, R. D. Pan, B. Teter, J. Cipolla, E. Chang, L. Z. Avila, S. K. Basak, E. S. Srivatsan, M. B. Wang, G. M. Cole, S. A. Frautschy, P. D. Hampton, K. F. Faull, Analytical Biochemistry, 2020, 596, 113636). Details of the NMR spectral assignment could also be found in the SDBS database AIST:Spectral abase for Organic Compounds, SDBS).

TABLE 3
Tabulated ¹H and ¹³C NMR data for curcumin (refer to FIG. 7 for numbering scheme)

¹H NMR (neat melt at 50° C., flame sealed d₆-dmso capillary used for lock, 400 MHz):
7.20 (d, 2H, H^{a/b}, ³J_{a-b(trans)} = 15.32 Hz), 6.75 (app s, 2H, H^{g}), 6.72 (app d, 2H, H^{e/d}, ³J_{e-d(ortho)} = 8.42 Hz*), 6.49 (app d, 2H,
H^{d/e}, ³J_{e-d(ortho)} = 8.16 Hz*), 6.13 (d, 2H, H^{b/a}, ³J_{a-b(trans)} = 15.32 Hz), 5.44 (s, 1H, H^{z})
*³J values observed for each multiplet are shown, the averaged ³J_{e-d} = 8.29 Hz.

Observation of the signals assigned to curcumin in its monoolein solution confirms that the true solution forms in melt at 50° C.

### Simulations—Confirming Interactions and Spatial Arrangements

We have recently performed molecular dynamics simulations on pure lipids system in presence of water. These studies were also extended for curcumin added systems, whose results work in support of our experimental observations.

### Further Enhancement** of Curcumin Solubility—Use of Bile Salts

Bile salts are known to interact with lipids at molecular level and facilitate phase transition in them to ease their digestion—see Sadeghpour, A.; Rappolt, M.; Misra, S.; Kulkarni, C. V., Bile Salts Caught in the Act: From Emulsification to Nanostructural Reorganization of Lipid Self-Assemblies. Langmuir 2018, 34 (45), 13626-13637. To further enhance the solubility of curcumin in lipid and lipid-water systems, we added bile salt mixture in various proportions. These studies are promising.

### Example 2—Curcumin Formulation (F02): Hydrated Lipid Systems

Formulations described in Table 1 above (F01-MO and F01-DU) can work in their current form, primarily for applications with topical or external modes of delivery. However, for internal use, an aqueous system is inevitable, which also means the curcumin loading must be studied under these conditions. We have employed F01-MO and F01-DU to prepare corresponding aqueous forms F02-MO and F02-DU as described below.

### Preparation

Formulation F01 containing curcumin, prepared above, was topped with additional amount of curcumin.
35-40% water (by weight) was added to these mixtures. Spatula or similar tools were used to blend the constituents until homogeneous.
Mixtures were heated (to 40-60° C.) using block heater. Spatula or similar tools were used to blend the constituents until homogeneous.
Mixtures were heated again, if required, followed by homogenization by spatula.
Final formulation (F02) was stored at room temperature (around 20° C.).

Note that, the sequence of preparation of F02 is important: first add required amount of curcumin to dry molten lipid, mix it with spatula and then add appropriate amount of water. If curcumin is added to preformed highly viscous cubic phase, it entails extra efforts (several free-thaw cycles) to obtain homogeneous formulation.

### Curcumin Solubility for F02-MO and F02-DU.

The maximum solubility achieved in F02 for MO was 15±4.0 mg/g (Table 1), whereas it was 25±5 mg/g for DU (FIG. 41A-D). Solubilized curcumin remains in this form for several weeks at room temperature. In presence of excess water (>35 wt/wt %) both MO and DU form bicontinuous cubic Pn3m phase, which due to its isotropic nature imparts inherent transparency to the formulations. If cooled below 20±3° C., small amount of lipid crystallizes into crystalline lamellar phase (L_{c}), which has opaque solid appearance. The formulation regains its transparency (FIG. 41A) upon gentle heating above 30° C. A very small amount of curcumin was found to sediment at the bottom of the vial when F02-DU was subjected to centrifugation at 3000 rpm for 5 min at room temperature. However, there was no any sedimentation after centrifugation for concentrations below 7.5±1.5 mg/g (Table 1).

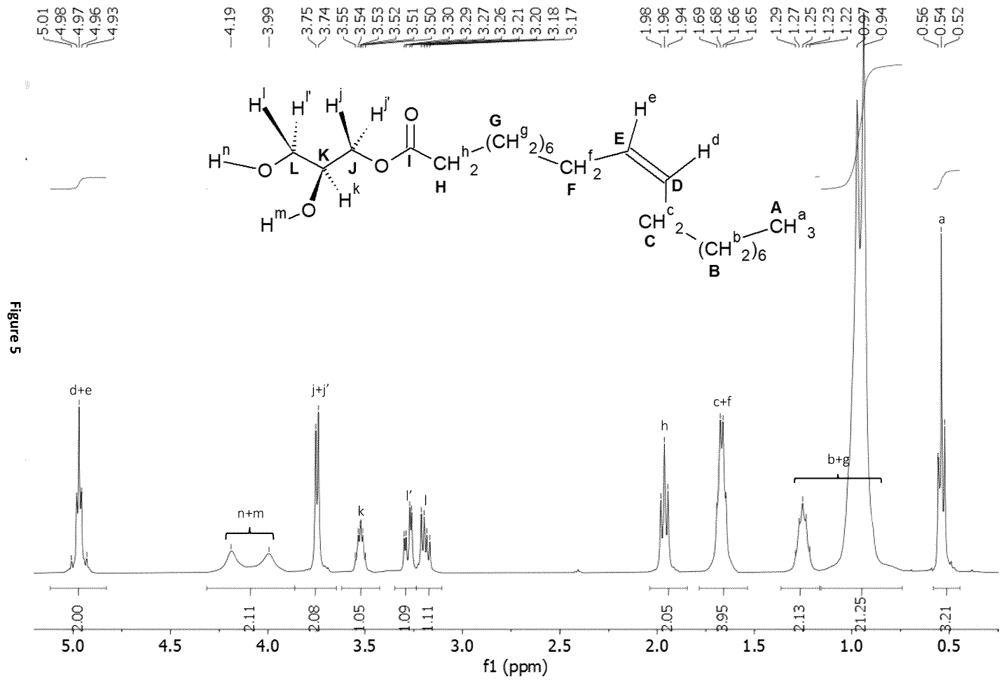

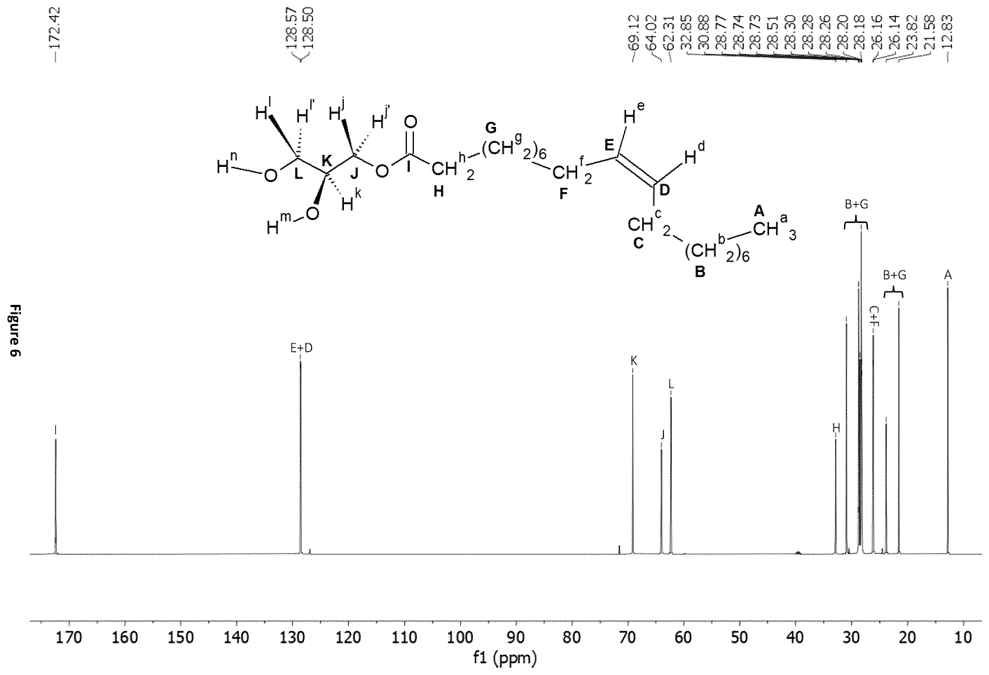

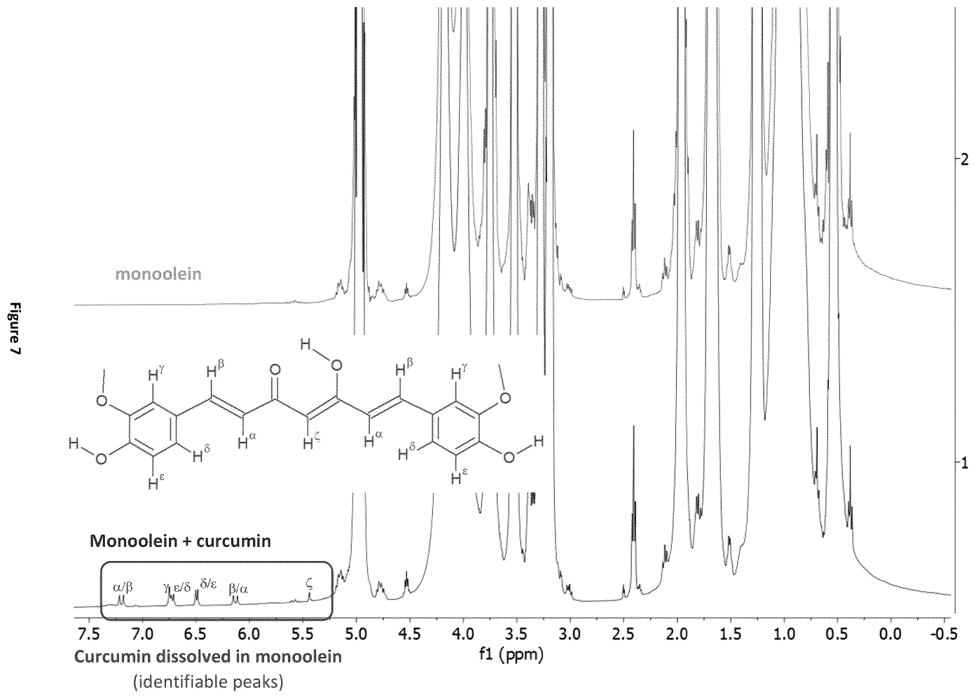

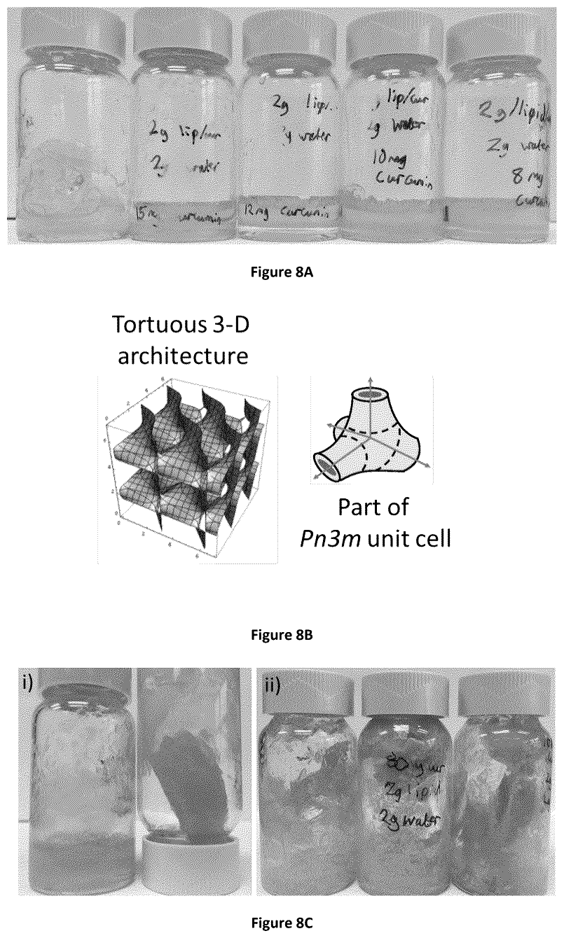

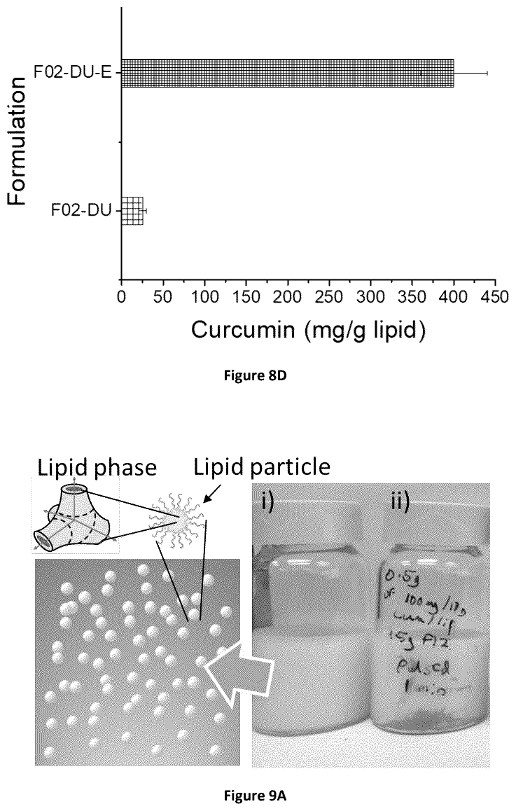

FIG. 8 relates to Formulation F02 (curcumin in DU and water) and shows A) Various concentrations (from L to R 10.0, 7.5, 6, 5 and 4.0 mg/g) of curcumin solubilized in DU-water cubic phase; B) Schematic diagram depicting the 3-dimensional tortuous architecture (L) formed of lipid bilayers in bicontinuous cubic Pn3m phase (R); C) Encapsulation of curcumin (concentrations shown from L to R: i) 100, 200 mg/g and ii) 200, 400 and 500 mg/g); D) Histogram showing the curcumin concentrations in formulation F02 with error bars.

### Curcumin Encapsulation

Further addition of curcumin above the maximum solubility point (25.0±5.0 mg/g for F02-DU), facilitates its encapsulation within the lipid system. The phase stays transparent until about 200 mg/g above which it starts becoming opaque and remains like this even above the MP of DU. As high as 400±40 mg/g (FIGS. 8C and 8D) curcumin gets encapsulated in F02-DU without sedimentation at room temperature or at 40° C. (well above MP of DU).

### Proposed Mechanism of Solubilization and Encapsulation

Intermolecular interactions between curcumin and lipid are perhaps similar to the F01 but water may also aid in hydrogen bonding, and therefore more curcumin is solubilized in aqueous cubic phases. Three-dimensional tortuous architecture (FIG. 8B) and characteristic high viscosity of lipid cubic phase as compared to lamellar or fluid isotropic phases further helps in encapsulating very high (400 mg/g) of curcumin in F02.

### Example 3—Curcumin Formulation (F03-DU): Nanostructured Dispersion (F03-DU-L and F03-DU-H)

Formulation F03 is in aqueous form, so it is suitable for external as well as internal modes of curcumin delivery. This formulation, however, benefits from its dispersed form as it retains the solubility properties of F02, but in addition has low viscosity making it more applicable, for instance, for an oral delivery.

These dispersions are essentially oil-in-water (O/W) or water-in-oil (W/O) emulsions, where an oil phase contains curcumin loaded lipid or lipid+additive and water phase contains water or a stabilizer dissolved in water. The dispersions are called ‘nanostructured’ because their morphology underlays liquid crystalline lipid phase (nanostructure).

### Preparation Method for F03-DU-L Nanostructured Dispersions

500 mg of Formulation F02 containing curcumin, was taken in a 20 ml vial.
It was topped with 9.5 g of pluronic F127 stabilizer (polyethylene glycol—i.e. PEG-based surfactant) solution (0.5% or 1 wt % in water).
   i. Instead of Pluronic F127, other non PEG-based surfactant (e.g. saponin) can be used to prepare this formulation;
   ii. Concentration of F127 can be varied from 0.5% up to 10% by weight in water.
Mixture was ultra-sonicated (Sonics & Materials Vibra-Cell VCX750, Jencons, UK) for 2 to 5 minutes with 10 sec pulse and 10 sec delay using 35% of the maximum power.
Sample was warm (about 40° C.), so allowed to cool to room temperature; resulting nanostructured emulsion was homogeneous and stable without any lipid lumps.
It was stored at room temperature (around 20° C.).

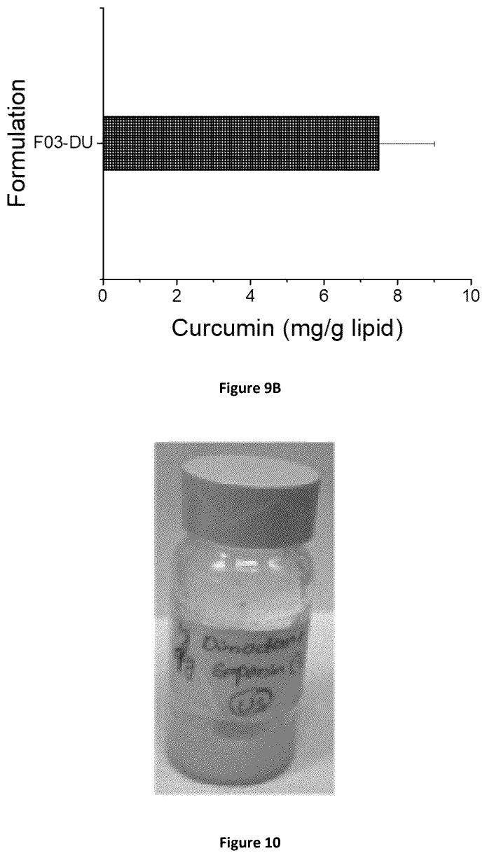

FIG. 9 relates to Formulation F03 (curcumin in nanostructured dispersion) and shows: A) Formulation 02 was ultrasonicated in presence of F127 solution, resulting into nanostructured dispersion (an emulsion in this case) After centrifugation, formulations containing >7.5 mg/g curcumin displayed a little sedimentation (as in ii)) but below this concentration formulation F03 were stable and homogeneous (as in i); B) Histogram showing the curcumin concentrations in formulation F03 with error bars.

### Preparation Method for F03-DU-H Nanostructured Dispersions

The ratio of curcumin containing lipid phase (or Formulation 02) to F127 solution can be varied in a broad range 2-60%:98-40% while preparing F03-DU.

A majority of O/W and W/O emulsions (made using FD02) can be prepared using lab-based vortex machine instead of ultra-sonication technique, particularly when the lipid phase content is above 15 Wt %. The latter were named as F03-DU-H (due to high concentration of lipid phase).
i. Concentrations above 15% of lipid phase usually exhibit cream/paste-like consistency (FIG. 10).
ii. Instead of Pluronic F127, other non-PEG-based surfactant (e.g. saponin) can be used to prepare this formulation.
iii. Concentration of F127 can be varied from 0.5% up to 10% by weight in water.

FIG. 10 shows an emulsion stabilized by using non-PEG based surfactant-saponin.

### Curcumin Solubility for F03-DU.

The maximum solubility achieved in F03-DU-L was 7.5±1.5 mg/g (FIG. 9A/B), which was same as found in F02 after centrifugation test (see above). Solubilized curcumin remains in this form for several weeks at room temperature.

For low lipid phase containing F03-DU-L dispersions no further curcumin could be solubilized or encapsulated as the excess curcumin got sedimented at the bottom of the vials (FIG. 9A(i)). But more curcumin (up to about 80.0±15 mg/g of lipid phase) could be loaded in high lipid phase containing F03-DU-H emulsions (usually above 15%) (FIG. 10), preferably the ones containing natural oil as additives.

Mechanism of solubilization was same as F02-DU (see above), as it was used as a precursor to prepare F03. Block co-polymer pluronic F127 was used as an interfacial stabilizer for stabilizing fragmented cubic phase particles within the dispersion.

### Example 4—Gel for Topical Application Based on Curcumin Solubilization in Dry Lipid System

A gel for topical application can be prepared based on Example 1 (see above), in particular based on the product shown in FIGS. 1A and 1B.

### Example 5: Gel for Topical Application Based on Curcumin Encapsulation in Dry Lipid System

A gel for topical application can be prepared based on Example 1 (see above), in particular based on the product shown in FIG. 1C.

### Example 6: Gel for Topical Application Based on Curcumin Solubilization in Hydrated Lipid System

A gel for topical application can be prepared based on Example 2 (see above), in particular based on the product shown in FIGS. 8A and 8B.

### Example 7: Gel for Topical Application Based on Curcumin Encapsulation in Hydrated Lipid System

A gel for topical application can be prepared based on Example 2 (see above), in particular based on the product shown in FIG. 8C.

### Example 8: Fluid for Oral Dose Based on Curcumin in Oil-In-Water Emulsion

A gel for topical application can be prepared based on Example 3 (see above), in particular based on the product shown in FIG. 9A.

O/W emulsions were also prepared using non-PEG based surfactant, saponin, as per FIG. 10.

### Example 9: Cream for Topical Application Based on Curcumin in Water-In-Oil Emulsion

Water-in-oil emulsions using surfactant stabilizers or without any stabilizers can be prepared and loaded with curcumin (FIG. 11)—such water-in-oil emulsions may be readily prepared (before adding curcumin) as per the literature procedures of Kulkarni, C. V.; Mezzenga, R.; Glatter, O., Water-in-oil nanostructured emulsions: towards the structural hierarchy of liquid crystalline materials. Soft Matter 2010, 6 (21), 5615-5624. Such water-in-oil emulsions are prepared from Example 2.

FIG. 11 shows water-in-oil emulsions prepared using various lipid combinations. These can be loaded with curcumin to obtain creams/pastes of various viscosities as shown on the right.

### Example 10: Fluid for Either Topical Application or an Oral Dose, Based on Curcumin Solubilization in Lipid-Bile Salt System (without and with Water)

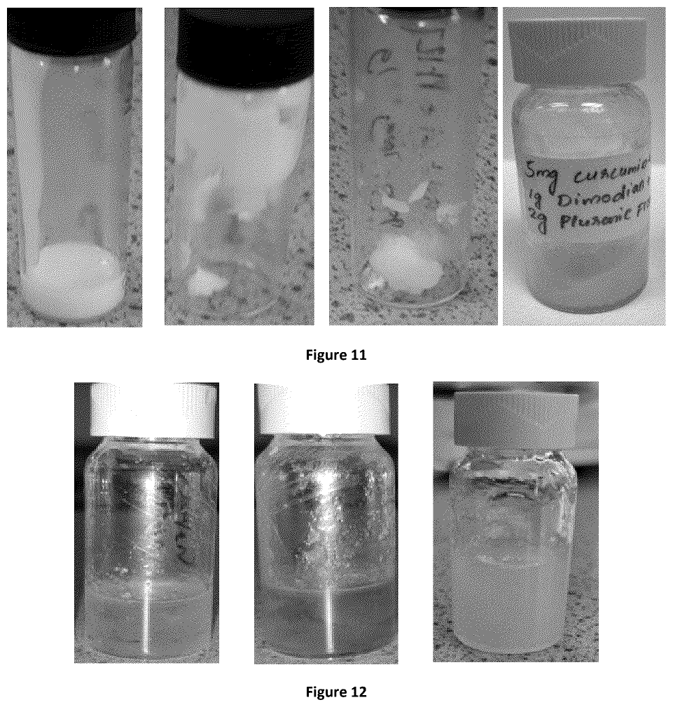

Previously we have shown that bile salt can act as a stabilizer for oil-in-water emulsions, however it sometimes changes the nanostructural phase of the lipid-water system—see Sadeghpour, A.; Rappolt, M.; Misra, S.; Kulkarni, C. V., Bile Salts Caught in the Act: From Emulsification to Nanostructural Reorganization of Lipid Self-Assemblies. Langmuir 2018, 34 (45), 13626-13637. Here we prepared a series of lipid-bile salt mixtures without and with water and loaded curcumin in them (FIG. 12). Due to high bile salt concentration curcumin seems to change its colour to orange/dark yellow (which is known to happen at high pH, changes native curcumin). These formulations can be further modified and optimized for solubilization and retaining the stability of curcumin in native form. This is prepared in a similar manner to Example 3.

FIG. 12 shows curcumin loaded bile salt-lipid mixtures (Left and middle) and bile salt-lipid-water mixtures (right).

### Example 11: Cream for Topical Application Based on Curcumin Solubilization in Lipid-Natural Oil (Morinpa Oil) System

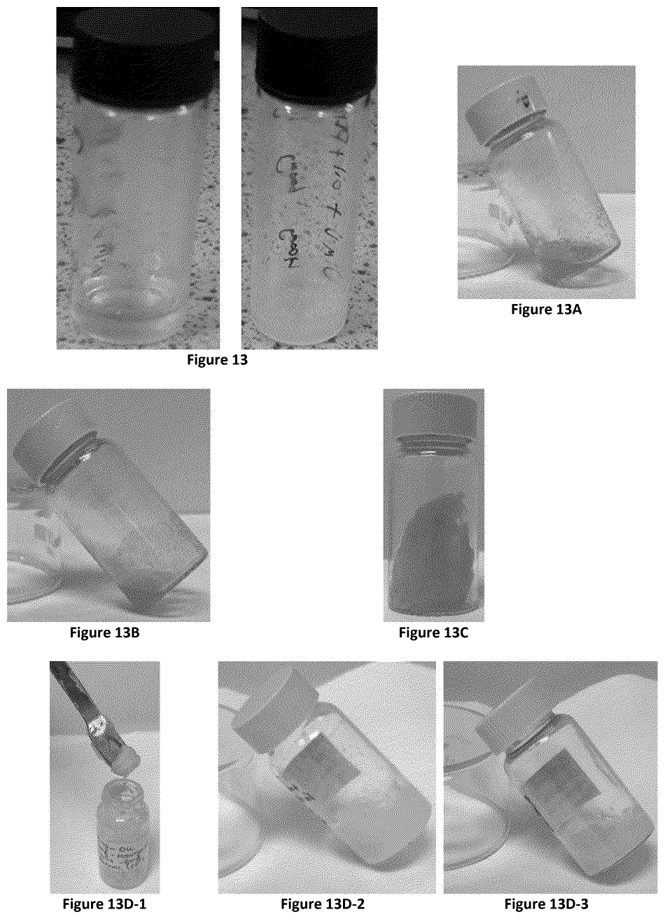

Moringa oil has extensive range of medicinal applications itself—see Gopalakrishnan, L.; Doriya, K.; Kumar, D. S., Moringa oleifera: A review on nutritive importance and its medicinal application. Food Science and Human Wellness 2016, 5 (2), 49-56. We can combine curcumin with the moringa oil and prepare various oral as well as topical formulations, similar to the ones in FIG. 13. Such water-in-oil emulsions are prepared from Example 2.

FIG. 13 shows Lipid formulations containing Moringa oil.

### Example 11a: Fluid or Gel for Topical Application Based on Curcumin Solubilization in Dry Lipid-Moringa Oil System

A gel for topical application can be prepared based on Example 1 (see above) but with an additional oil component, in this case moringa oil. FIG. 13A shows a lipid formulation containing 0.7936 wt % curcumin, 49.96 wt % DU, and 49.96 wt % moringa oil (i.e. 50:50 DU/oil). FIG. 13B shows a lipid formulation containing 0.7936 wt % curcumin, 89.28 wt % DU, and 9.92% moringa oil (i.e. 90:10 DU/oil). Moringa oil has its own nutritional and health benefits (Gopalakrishnan, L., K. Doriya, and D. S. Kumar, Moringa oleifera: A review on nutritive importance and its medicinal application. Food Science and Human Wellness, 2016. 5(2): p. 49-56). Addition of moringa oil further helps in controlling the physical form of curcumin formulations. The DU-moringa oil ratio can be varied widely whilst still yielding the benefits of the invention.

### Example 11b: Fluid or Gel for Topical Application Based on Curcumin Solubilization in Hydrated Lipid-Moringa Oil System

A gel for topical application can be prepared based on Example 2 (see above) but with an additional oil component, in this case moringa oil. FIG. 13C shows a lipid formulation containing 2.5341 wt % curcumin, 58.48 wt % DU, 6.498 wt % moringa oil, and 32.489 wt % water (i.e. 90:10 DU/oil). The DU-moringa oil ratio can be varied widely whilst still yielding the benefits of the invention.

### Example 11c: Fluid for Oral Application or Cream for Topical Application Based on Curcumin Solubilization in Lipid-Moringa Oil System

The physical form of O/W and W/O emulsions can be controlled using addition of various concentrations of moringa oil. DU to moringa oil ratio can be varied widely in the form of, 90:10, 80:20, 60:40, etc. FIG. 13D-1 shows a lipid formulation containing 0.397 wt % curcumin, 44.597 wt % DU, 4.9554 wt % moringa oil, 49.554 wt % water, and 0.49554 wt % Pluronic F127 surfactant (i.e. 90:10 DU:oil). FIG. 13D-2 shows a lipid formulation containing 0.397 wt % curcumin, 39.64 wt % DU, 9.94 wt % moringa oil, 49.554 wt % water, and 0.4955 wt % F127 (i.e. 80:20 DU:oil). FIG. 13D-3 shows a lipid formulation containing 0.397 wt % curcumin, 29.73% DU, 19.82% moringa oil, 49.55 wt % water, and 0.4955 wt % F127 (i.e. 60:40 DU:oil). The DU-moringa oil ratio can be varied widely whilst still yielding the benefits of the invention.

### Example 12: Fluid for Oral Application or Cream for Topical Application Based on Curcumin Solubilization in Lipid-Coconut Oil System

The physical form of O/W and W/O emulsions can be controlled using addition of various concentrations of coconut oil. DU to coconut oil ratio can be varied widely in the form of, 90:10, 80:20, etc. Such water-in-oil emulsions are prepared from Example 2. (Composition of above 90:10 formulation was as follows: a=0.24%, b1=44.8%, b2=4.97%, c=49.50%, d=0.49%, where, a=curcumin, b1=DU, b2=coconut oil, c=water and d=F127 stabilizer).

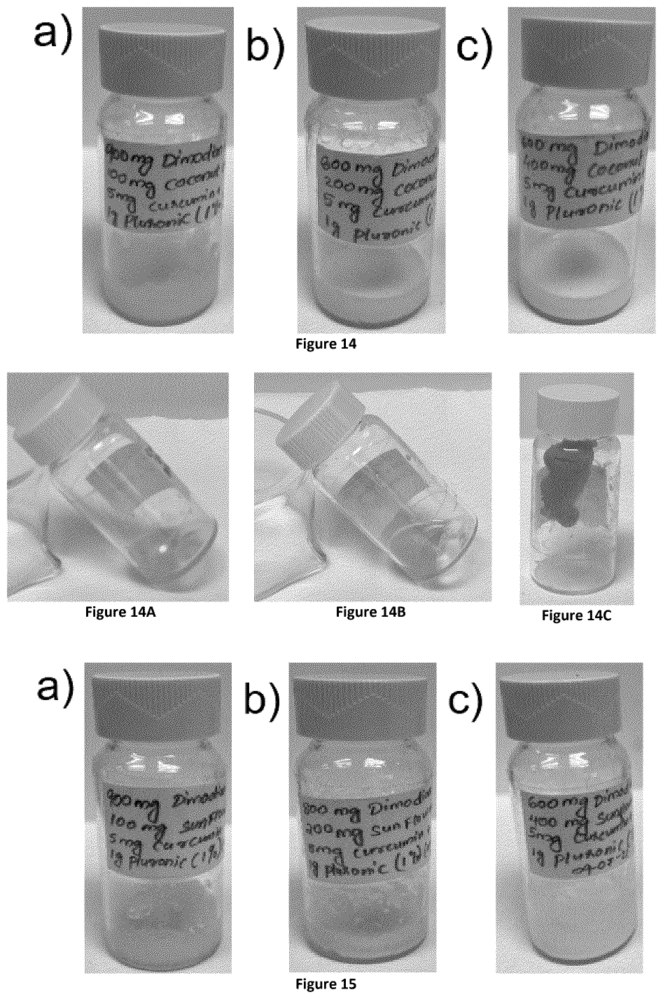

FIG. 14 shows lipid formulations containing DU:Coconut oil in the following proportions a) 90:10, b) 80:20 and c) 60:40.

### Example 12a: Fluid or Gel for Topical Application Based on Curcumin Solubilization in Dry Lipid-Coconut Oil System

A gel fortopical application can be prepared based on Example 1 (see above) but with an additional oil component, in this case coconut oil. FIG. 14A shows a lipid formulation containing 0.398 wt % curcumin, 49.8 wt % DU, and 49.8 wt % coconut oil (i.e. 50:50 DU/oil). FIG. 14B shows a lipid formulation containing 0.596 wt % curcumin, 89.46 wt % DU, and 9.94% coconut oil (i.e. 90:10 DU/oil). Coconut oil has its own nutritional and health benefits (Wallace, T. C., Health Effects of Coconut Oil-A Narrative Review of Current Evidence. J Am Coll Nutr, 2019. 38(2): p. 97-107). Addition of coconut oil further helps in controlling the physical form of curcumin formulations. The DU-coconut oil ratio can be varied widely whilst still yielding the benefits of the invention.

### Example 12b: Fluid or Gel for Topical Application Based on Curcumin Solubilization in Hydrated Lipid-Coconut Oil System

A gel for topical application can be prepared based on Example 2 (see above) but with an additional oil component, in this case coconut oil. FIG. 14C shows a lipid formulation containing 1.96 wt % curcumin, 58.82 wt % DU, 6.535 wt % coconut oil, and 32.679 wt % water (i.e. 90:10 DU/oil). The DU-coconut oil ratio can be varied widely whilst still yielding the benefits of the invention.

### Example 13: Fluid for Oral Application or Cream for Topical Application Based on Curcumin Solubilization in Lipid-Sunflower Oil System

The physical form of O/W and W/O emulsions can be controlled using addition of various concentrations of sunflower oil. DU to sunflower oil ratio can be varied widely in the form of, 90:10, 80:20, etc. (Composition of above 90:10 formulation was as follows: a=3.47%, b1=43.33%, b2=4.82%, c=47.90%, d=0.48%, where, a=curcumin, b1=DU, b2=sunflower oil, c=water and d=F127 stabilizer).

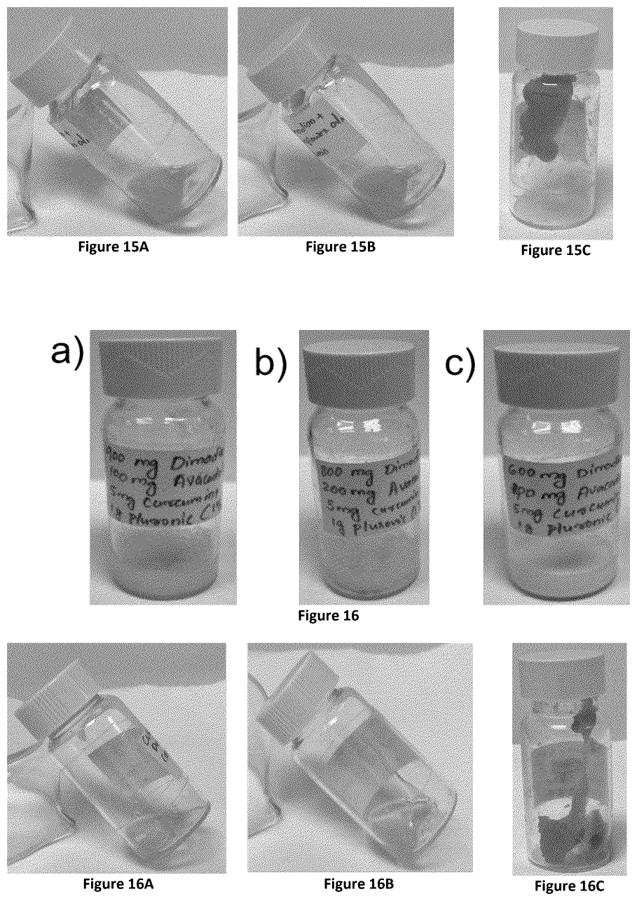

FIG. 15 shows lipid formulations containing DU:Sunflower oil in the following proportions a) 90:10, b) 80:20 and c) 60:40.

### Example 13a: Fluid or Gel for Topical Application Based on Curcumin Solubilization in Dry Lipid-Sunflower Oil System

A gel fortopical application can be prepared based on Example 1 (see above) but with an additional oil component, in this case sunflower oil. FIG. 15A shows a lipid formulation containing 0.398 wt % curcumin, 49.8 wt % DU, and 49.8 wt % sunflower oil (i.e. 50:50 DU/oil). FIG. 15B shows a lipid formulation containing 0.596 wt % curcumin, 89.46 wt % DU, and 9.94% sunflower oil (i.e. 90:10 DU/oil). Sunflower oil has its own nutritional and health benefits (Petraru, A., F. Ursachi, and S. Amariei, Nutritional Characteristics Assessment of Sunflower Seeds, Oil and Cake. Perspective of Using Sunflower Oilcakes as a Functional Ingredient. Plants (Basel), 2021. 10(11)). Addition of sunflower oil further helps in controlling the physical form of curcumin formulations. The DU-sunflower oil ratio can be varied widely whilst still yielding the benefits of the invention.

### Example 13b: Fluid or Gel for Topical Application Based on Curcumin Solubilization in Hydrated Lipid-Sunflower Oil System

A gel for topical application can be prepared based on Example 2 (see above) but with an additional oil component, in this case sunflower oil. FIG. 15C shows a lipid formulation containing 1.96 wt % curcumin, 58.82 wt % DU, 6.535 wt % sunflower oil, and 32.679 wt % water (i.e. 90:10 DU/oil). The DU-sunflower oil ratio can be varied widely whilst still yielding the benefits of the invention.

### Example 14: Fluid for Oral Application or Cream for Topical Application Based on Curcumin Solubilization in Lipid-Avocado Oil System

The physical form of O/W and W/O emulsions can be controlled using addition of various concentrations of Avocado oil. DU to Avocado oil ratio can be varied widely in the form of, 90:10, 80:20, etc. (Composition of above 90:10 formulation was as follows: a=3.47%, b1=43.33%, b2=4.82%, c=47.90%, d=0.48%, where, a=curcumin, b1=DU, b2=avocado oil, c=water and d=F127 stabilizer).

FIG. 16 shows lipid formulations containing DU:Avocado oil in the following proportions a) 90:10, b) 80:20 and c) 60:40.

### Example 14a: Fluid or Gel for Topical Application Based on Curcumin Solubilization in Dry Lipid-Avocado Oil System

A gel for topical application can be prepared based on Example 1 (see above) but with an additional oil component, in this case avocado oil. FIG. 16A shows a lipid formulation containing 0.398 wt % curcumin, 49.8 wt % DU, and 49.8 wt % avocado oil (i.e. 50:50 DU/oil). FIG. 16B shows a lipid formulation containing 0.596 wt % curcumin, 89.46 wt % DU, and 9.94% avocado oil (i.e. 90:10 DU/oil). Avocado oil has its own nutritional and health benefits (Flores, M., et al., Avocado Oil: Characteristics, Properties, and Applications. Molecules, 2019. 24(11)). Addition of avocado oil further helps in controlling the physical form of curcumin formulations. The DU-avocado oil ratio can be varied widely whilst still yielding the benefits of the invention.

### Example 14b: Fluid or Gel for Topical Application Based on Curcumin Solubilization in Hydrated Lipid-Avocado Oil System

A gel for topical application can be prepared based on Example 2 (see above) but with an additional oil component, in this case avocado oil. FIG. 16C shows a lipid formulation containing 1.96 wt % curcumin, 58.82 wt % DU, 6.535 wt % avocado oil, and 32.679 wt % water (i.e. 90:10 DU/oil). The DU-avocado oil ratio can be varied widely whilst still yielding the benefits of the invention.

### Example 15: Wound Healing Bandages, Oral Application, and Topic Applications, Based on Lipid-Hydrogel Hydrogels, Films, Pastilles or Gellies

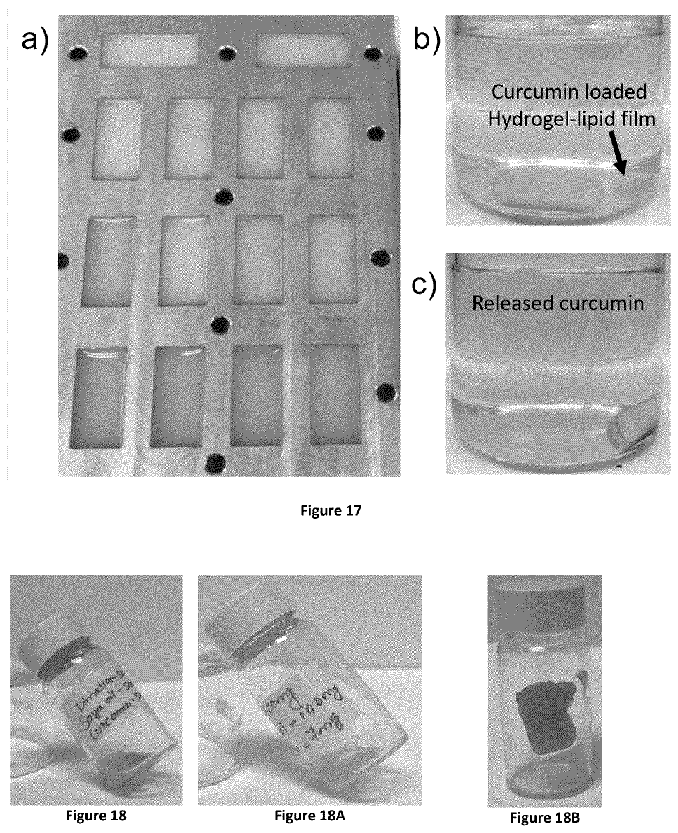

We are able to formulate (with addition/optimization of additives) curcumin-lipid systems into edible pastilles or jellies (FIG. 17). These can be prepared from hydrogels and films using the protocol reported in Kulkarni, C. V.; Moinuddin, Z.; Patil-Sen, Y.; Littlefield, R.; Hood, M., Lipid-hydrogel films for sustained drug release. International Journal of Pharmaceutics 2015, 479, 416-421. Also, incorporating the curcumin loaded lipid particles into hydrogel matrix offers sustained release of curcumin over long period.

FIG. 17 shows: a) Film mould to prepare curcumin loaded hydrogel-lipid films b) Drug release set-up containing curcumin loaded film c) released curcumin from hydrogel-lipid film.

Various films were prepared (as shown in FIG. 17a) by mixing 5 gm of 4 wt % hydrogel (K-carrageenan) in water with the same weight of curcumin loaded cubosomes (prepared from either of 2.5%, 5%, 10% lipid in 0.5% F127 solution), pouring in the moulds in FIG. 17a and drying at room temperature. Release of curcumin was monitored in aqueous and physiological buffer solutions using UV-Vis spectroscopy.

### Example 16: Fluid or Gel for Topical Application Based on Curcumin Solubilization in Dry Lipid-Soya Oil System

A gel for topical application can be prepared based on Example 1 (see above) but with an additional oil component, in this case soya oil. FIG. 18 shows a lipid formulation containing 0.497 wt % curcumin, 49.75 wt % DU, and 49.75% soya oil. FIG. 18A shows a lipid formulation containing 0.695 wt % curcumin, 89.37 wt % DU, and 9.93 wt % soya oil. Soya oil has its own nutritional and health benefits (Messina, M., G. Shearer, and K. Petersen, Soybean oil lowers circulating cholesterol levels and coronary heart disease risk, and has no effect on markers of inflammation and oxidation. Nutrition, 2021. 89: p. 111343), and curcumin itself is soluble in soya oil (Takenaka, M., et al., Effective Extraction of Curcuminoids by Grinding Turmeric (Curcuma longa) with Medium-chain Triacylglycerols. Food Science and Technology Research, 2013. 19: p. 655-659). Addition of soya oil further helps in controlling the physical form of curcumin formulations. The DU-soya oil ratio can be varied widely whilst still yielding the benefits of the invention.

### Example 16a: Fluid or Gel for Topical Application Based on Curcumin Solubilization in Hydrated Lipid-Soya Oil System

A gel for topical application can be prepared based on Example 2 (see above) but with an additional oil component, in this case soya oil. FIG. 18B shows a lipid formulation containing 2.344 wt % curcumin, 58.59 wt % DU, 6.51% soya oil, 32.55 wt % water (i.e. 90:10 DU/oil). The DU-soya oil ratio can be varied widely whilst still yielding the benefits of the invention.

### Example 16b: Fluid for Oral Application or Cream for Topical Application Based on Curcumin Solubilization in Lipid-Soya Oil System

The physical form of O/W and W/O emulsions can be controlled using addition of various concentrations of soya oil. DU to soya oil ratio can be varied widely in the form of, 90:10, 80:20, 60:40, etc. FIG. 18C-1 shows a lipid formulation containing 0.347 wt % curcumin, 44.62 wt % DU, 4.958 wt % soya oil, 49.58 wt % water, 0.496 wt % F127. FIG. 18C-2 shows a lipid formulation containing 0.347 wt % curcumin, 39.667 wt % DU, 9.916 wt % soya oil, 49.58 wt % water, 0.496 wt % F127. FIG. 18C-3 shows a lipid formulation containing 0.347 wt % curcumin, 29.75 wt % DU, 19.83 wt % soya oil, 49.58 wt % water, 0.496 wt % F127.

### Example 17: Fluid or Gel for Topical Application Based on Curcumin Solubilization in Dry Lipid-Corn Oil System

A gel fortopical application can be prepared based on Example 1 (see above) but with an additional oil component, in this case corn oil. FIG. 19 shows a lipid formulation containing 0.497 wt % curcumin, 49.75 wt % DU, and 49.75% corn oil. FIG. 19A shows a lipid formulation containing 0.695 wt % curcumin, 89.37 wt % DU, and 9.93 wt % corn oil. Corn oil has its own nutritional and health benefits (Maki, K. C., et al., Corn Oil Lowers Plasma Cholesterol Compared with Coconut Oil in Adults with Above-Desirable Levels of Cholesterol in a Randomized Crossover Trial. J Nutr, 2018. 148(10): p. 1556-1563), and curcumin itself is soluble in corn oil (Takenaka, M., et al., Effective Extraction of Curcuminoids by Grinding Turmeric (Curcuma longa) with Medium-chain Triacylglycerols. Food Science and Technology Research, 2013. 19: p. 655-659). Addition of corn oil further helps in controlling the physical form of curcumin formulations. The DU-corn oil ratio can be varied widely whilst still yielding the benefits of the invention.

### Example 17a: Fluid or Gel for Topical Application Based on Curcumin Solubilization in Hydrated Lipid-Corn Oil System

A gel for topical application can be prepared based on Example 2 (see above) but with an additional oil component, in this case corn oil. FIG. 19B shows a lipid formulation containing 2.344 wt % curcumin, 58.59 wt % DU, 6.51% corn oil, 32.55 wt % water. The DU-corn oil ratio can be varied widely whilst still yielding the benefits of the invention.

### Example 17b: Fluid for Oral Application or Cream for Topical Application Based on Curcumin Solubilization in Lipid-Corn Oil System

The physical form of O/W and W/O emulsions can be controlled using addition of various concentrations of corn oil. DU to corn oil ratio can be varied widely in the form of, 90:10, 80:20, 60:40, etc. FIG. 19C-1 shows a lipid formulation containing 0.347 wt % curcumin, 44.62 wt % DU, 4.958 wt % corn oil, 49.58 wt % water, 0.496 wt % F127. FIG. 19C-2 shows a lipid formulation containing 0.347 wt % curcumin, 39.667 wt % DU, 9.916 wt % corn oil, 49.58 wt % water, 0.496 wt % F127. FIG. 19C-3 shows a lipid formulation containing 0.347 wt % curcumin, 29.75 wt % DU, 19.83 wt % corn oil, 49.58 wt % water, 0.496 wt % F127.

### Example 18: Gel for Topical Application Based on Curcumin (in the Form of Turmeric) Solubilization in Dry Lipid System

A gel for topical application can be prepared based on Example 1 (see above), by using raw turmeric (as the curcumin source). As such, the final product contains additional ingredients contained within the turmeric. Turmeric (Curcuma longa) is a prime source of curcumin but it also consists of proteins, carbohydrates, oils, lipids, and other substances (El-Saadony, M. T., et al., Impacts of turmeric and its principal bioactive curcumin on human health: Pharmaceutical, medicinal, and food applications: A comprehensive review. Frontiers in Nutrition, 2023. 9). The turmeric (without extracting curcumin) was completely soluble in DU (up to 7 mg/g of DU). By way of example. FIG. 20 shows a lipid formulation containing 0.027 wt % curcumin, 99.3 wt % DU, 0.667 wt % other substances present in the turmeric.

### Example 19: Gel for Topical Application Based on Curcumin (in the Form of Turmeric) Solubilization in Hydrated Lipid System

A gel for topical application can be prepared based on Example 2 (see above), by using raw turmeric (as the curcumin source). As such, the final product contains additional ingredients contained within the turmeric. The turmeric (without extracting curcumin) was completely soluble in the hydrated lipid system (up to 23 mg/g of lipid cubic phase formed of 60:40 ratio of DU:water). By way of example, FIG. 21 shows a lipid formulation containing 0.0895 wt % curcumin, 58.65 wt %, DU, 39.104 wt % water, 2.15 wt % other substances present in turmeric.

### Example 20: Fluid or Gel for Topical Application Based on Curcumin Solubilization in Dry Lipid-Aloe vera Gel System

Preparation of Aloe vera gel: fresh Aloe vera (Aloe barbadensis miller) leaves were used to prepare the gel. The leaves were washed with distilled water to remove the dirt and water-soluble yellow stain (the resin containing the latex). The thick part (green coloured outer part) was peeled off to collect the transparent gel. This gel was then soaked in a cold water for about 30 minutes to remove remaining yellow stain (this can cause skin irritation). Washed gel was blended until it becomes fluid and frothy.

Separately, 3 g of xanthan gum (thickening agent) was dispersed in 1 g of vegetable glycerol (dispersion of xanthan gum in vegetable glycerol aids its dispersion in water; otherwise, it forms lumps with water due to its fast-hydrating nature). This mixture was then poured slowly and with stirring to the 100 g of blended aloe vera gel; stirring continued until it becomes homogeneous (it takes about 10-15 minutes; stirring with spatula gives better result than with the magnetic stirrer). The final mixture was allowed to stand for about 30 minutes to obtain a thickened translucent gel.

A gel for topical application can be prepared by mixing DU and aloe vera gel in 1:1 ratio and loading it with curcumin. Aloe vera gel is known to facilitate healthy skin, not least due to its richness in vitamin A, C, E and B12. It is also known for its anti-inflammatory anti-ageing, and anti-bacterial properties (Sajjad, A. and S. Subhani Sajjad, <i>Aloe vera</i>: An Ancient Herb for Modern Dentistry—A Literature Review. Journal of Dental Surgery, 2014. 2014: p. 210463). By way of example, FIG. 22 shows a lipid formulation containing 1.88 wt % curcumin, 47.16 wt % DU, 47.16 wt % aloe vera gel, 3.77 wt % xanthan gum and vegetable glycerol.

### Example 21: Gel for Topical Application Based on Curcumin (in the Form of Curcuminoids) Solubilization in Dry Lipid Sem

### 1. Extracted Curcuminoids Formulations

Curcuminoids were extracted in the laboratory using solvent extraction method (Heffernan, C., et al., Extraction and Purification of Curcuminoids from Crude Curcumin by a Combination of Crystallization and Chromatography. Organic Process Research & Development, 2017. 21(6): p. 821-826). The extracted curcuminoids contain curcumin, demethoxycurcumin, and bis-demethoxycurcumin as confirmed by thin-layer chromatography (TLC), Nuclear magnetic resonance (NMR) and TLC-MS (mass spectroscopy). The curcuminoids (without extracting curcumin) were completely soluble in a lipid system (up to 650 mg/g of DU). Curcuminoids generally contain 60-70% of curcumin (Dei Cas, M. and R. Ghidoni, Dietary Curcumin: Correlation between Bioavailability and Health Potential. Nutrients, 2019. 11(9)).

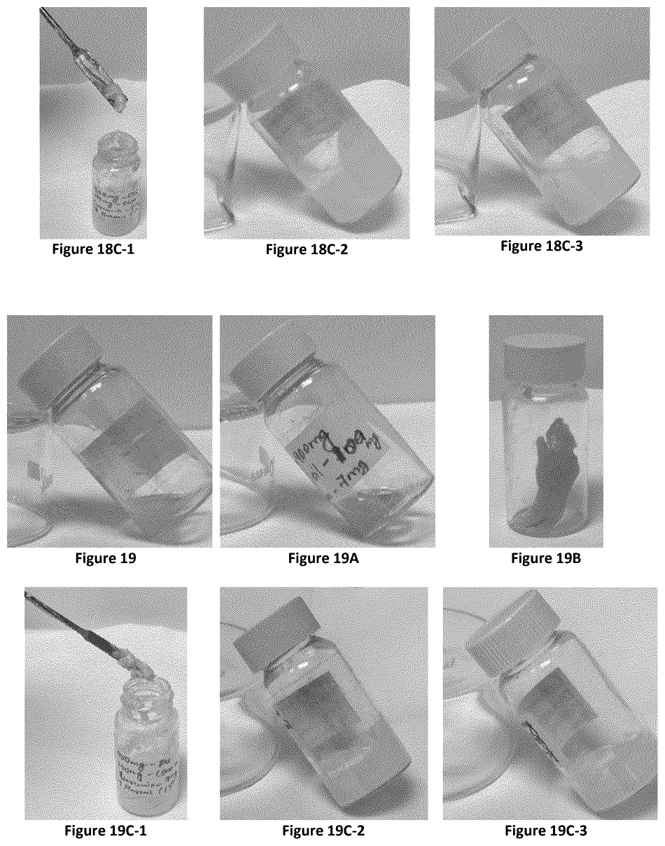

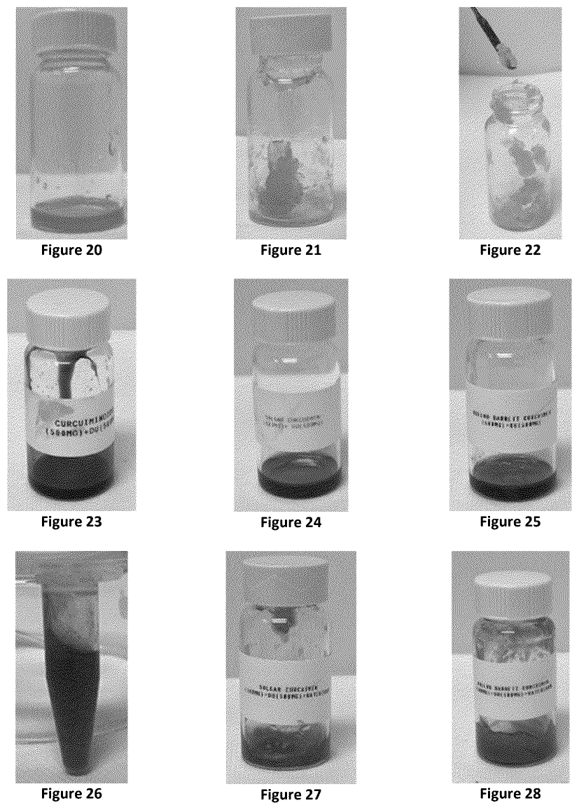

A gel for topical application can be prepared based on Example 1 (see above), using the aforementioned extracted curcuminoid mixture. By way of example, FIG. 23 shows a lipid formulation containing 32.5 wt % curcumin, 50 wt % DU, 17.5 wt % demethoxycurcumin, and bis-demethoxycurcumin.

### 2. Commercial Curcuminoids Formulations—Solgar

Commercial curcuminoids capsules, Solgar Full Spectrum Curcumin (turmeric extract), were purchased from Holland & Barrett, Preston, UK. Curcuminoids generally contain 60-70% of curcumin (Dei Cas, M. and R. Ghidoni, Dietary Curcumin: Correlation between Bioavailability and Health Potential. Nutrients, 2019. 11(9))—this information along with the compositional details provided on the products may be used to calculate amounts of ingredients in final formulations produced according to the invention. For instance, an 800 mg of Solgar capsule contains turmeric extract providing 40 mg of curcumin.

A gel for topical application can be prepared based on Example 1 (see above), by using the Solgar Full Spectrum Curcumin (turmeric extract) contained within the capsules as the source of curcumin. The turmeric extract (without extracting curcumin) was completely soluble in dry lipid system, and yielded the composition shown in FIG. 24. FIG. 24 shows a lipid formulation 1.95 wt % curcumin, 50 wt % DU, 48.05 wt % others, where others is components of (Solgar) turmeric extract other than curcumin.

### 3. Other COMMERCIAL Curcuminoids Formulations—East Meets West

Commercial curcuminoids capsules (East meets west) were purchased from Holland & Barrett, Preston, UK. The full name is “Holland & Barrett Novasol Bio-available Curcumin & Vit D 30 Capsules”. Curcuminoids generally contain 60-70% of curcumin—this information along with the compositional details provided on the products were used to make the following calculations. 500 mg of East meets West capsule contains novasol curcumin solution providing 30 mg of curcuminoids.

A gel for topical application can be prepared based on Example 1 (see above), by the using the formulation within the “Holland & Barrett Novasol Bio-available Curcumin & Vit D 30 Capsules” as the source of curcumin. brown liquid inside the capsule was completely soluble in dry lipid system, to yield the composition shown in FIG. 25. FIG. 25 shows a lipid formulation containing 2.5 wt % curcumin, 50 wt % DU, 47.5 wt % others, where others is the non-curcumin ingredients (demethoxycurcumin, bis-demethoxycurcumin, Vitamin D, polysorbate 80).

### Example 21a: Gel for Topical Application Based on Curcumin (in the Form of Curcuminoids) Solubilization in Hydrated Lipid Sem

### 1. Extracted Curcuminoids Formulations

A gel for topical application can be prepared based on Example 2 (see above), using the extracted curcuminoid mixture, detailed in Part 1 of Example 21 above, as the source of curcumin. This curcuminoid mixture was completely soluble in a hydrated lipid system. By way of example, FIG. 26 shows a lipid formulation containing 21.66 wt % curcumin, 40 wt % DU, 26.6 wt % water, 11.6 wt % others, where others is primarily demethoxycurcumin, and bis-demethoxycurcumin.

### 2. Commercial Curcuminoids Formulations—Solgar

A gel for topical application can be prepared based on Example 2 (see above), using the Solgar formulation, detailed in Part 2 of Example 21 above, as the source of curcumin. The Solgar turmeric extract was completely soluble in a hydrated lipid system. By way of example, FIG. 27 shows a lipid formulation containing 1.3 wt % curcumin, 40 wt % DU, 26.6 wt % water, 32.03 wt % others, where others includes components in turmeric extract other than curcumin.

### 3. Other Commercial Curcuminoids Formulations—East Meets West

A gel for topical application can be prepared based on Example 2 (see above), using the East Meets West formulation, detailed in Part 3 of Example 21 above, as the source of curcumin. The brown liquid inside the East Meets West capsules was completely soluble in a hydrated lipid system. By way of example, FIG. 28 shows a lipid formulation containing 1.67 wt % curcumin, 40 wt % DU, 26.6 wt % water, 31.66 wt % others, where others includes demethoxycurcumin, bis-demethoxycurcumin, Vitamin D, polysorbate 80.

### Example 21b: Dispersion of Curcumin in the Form of Curcuminoids

The following are examples of dispersions, essentially oil-in-water (O/W) emulsions, with an oil phase containing curcumin-loaded lipid is dispersed in a water phase. The dispersions are called ‘nanostructured’ because their morphology underlays liquid crystalline lipid phase (nanostructure). Curcuminoids (without extracting curcumin) were used to prepare the dispersion.

### 1. Extracted Curcuminoids Formulations

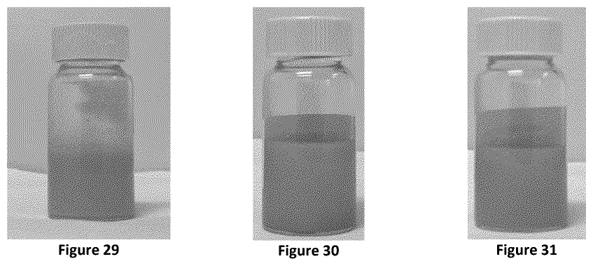

A dispersion, which may, for instance, serve as an orally administrable formulation or a cream for topical application, can be produced based on Example 3 (see above), using the extracted curcuminoid mixture, detailed in Part 1 of Example 21 above, as the source of curcumin. By way of example, FIG. 29 shows a lipid formulation (oil-in-water dispersion/emulsion) containing 1.89 wt % curcumin, 4.85 wt % DU, 92.23 wt % water, 1.02 wt % others, where others is primarily demethoxycurcumin, and bis-demethoxycurcumin.

### 2. Commercial Curcuminoids Formulations—Solgar

A dispersion, which may, for instance, serve as an orally administrable formulation or a cream for topical application, can be produced based on Example 3 (see above), using the Solgar formulation, detailed in Part 2 of Example 21 above, as the source of curcumin. By way of Example, FIG. 30 shows a lipid formulation (oil-in-water dispersion/emulsion) containing 0.95 wt % curcumin, 4.85 wt % DU, 92.23 wt % water, 1.97 wt % others (i.e. components in turmeric extract except curcumin)

### 3. Other Commercial Curcuminoids Formulations—East Meets West

A dispersion, which may, for instance, serve as an orally administrable formulation or a cream for topical application, can be produced based on Example 3 (see above), using the brown liquid inside the East Meets West capsules, detailed in Part 3 of Example 21 above, as the source of curcumin. By way of Example, FIG. 31 shows a lipid formulation (oil-in-water dispersion/emulsion) containing 1.114 wt % curcumin, 4.85 wt % DU, 92.23 wt % water, 2.799 wt % others (i.e. demethoxycurcumin, bis-demethoxycurcumin, Vitamin D, polysorbate 80).

### Numbered Paragraphs

The present invention, and embodiments thereof, may also be defined by way of the following numbered paragraphs.
1. A composition comprising a curcumin compound and one or more glycerides, wherein at least one of the glyceride(s) comprises at least one unsaturated moiety, preferably at least one alkene moiety, most preferably at least one cis-alkene moiety.
2. The composition as defined in paragraph 1, wherein the one or more glycerides are one or more monoglycerides bearing a C₈-C₂₀ fatty acid moiety having at least one cis-alkene.
3. The composition as defined in paragraph 2, wherein the one or more glycerides are a first monoglyceride and a second monoglyceride, each bearing a C₈-C₂₀ fatty acid moiety having at least one cis-alkene.
4. The composition as defined in paragraph 3, wherein the one or more glycerides are glyceryl monooleate and glyceryl monolinoleate.
5. The composition as defined in paragraph 4, wherein the glyceryl monooleate and glyceryl monolinoleate are present in a weight ratio between 10:90 and 90:10.
6. The composition as defined in any preceding paragraph, wherein the composition comprises 0.01-40 wt % curcumin compound.
7. The composition as defined in any preceding paragraph, wherein the composition comprises 0.1-30 wt % curcumin compound.
8. The composition as defined in any preceding paragraph, wherein the weight ratio of the curcumin compound to the glyceride(s) is between 0.5:1000 and 800:1000.
9. The composition as defined in paragraph 8, wherein the weight ratio of the curcumin compound to the glyceride(s) is between 30:1000 and 500:1000.
10. The composition as defined in any of paragraphs 1 to 7, wherein the weight ratio of the curcumin compound to the glyceride(s) is between 200:1000 and 600:1000.
11. The composition as defined in any preceding paragraph, wherein the stipulated glyceride(s) constitute at least 85 wt % of the total amount of lipids present within the composition.
12. The composition as defined in any preceding paragraph, wherein the composition comprises less than or equal to 60 wt % water.
13. The composition as defined in paragraph 12, wherein the composition comprises 10-60 wt % water.
14. The composition as defined in paragraph 12, wherein the composition has at most 2 wt % water.
15. The composition as defined in any preceding paragraph, wherein at least 50 wt % of the composition is constituted by the stipulated ingredients (curcumin compound, glyceride(s), any water that is present, and any additionally stipulated ingredients).
16. The composition as defined in any preceding paragraph, wherein centrifugation (e.g. 3000 rpm for 5 min at RT) of the composition yields less than 2 wt % sedimentation (i.e. less than 2 wt % of the composition as a whole).
17. The composition as defined in any preceding paragraph, wherein the composition is either free of poloxamer. polysorbate, and PEG-40 castor oil surfactants or comprises at most 1 wt % free of poloxamer. polysorbate, and PEG-40 castor oil surfactants.
18. The composition as defined in paragraph 17, wherein the composition is either free of surfactants or comprises at most 1 wt % surfactant(s).
19. The composition as defined in any preceding paragraph, wherein the composition is free of any compounds that comprise polyalkylene glycol ester moieties (e.g. polyethylene glycol and/or polypropylene glycol ester moieties), and suitably also free of polyvinyl alcohol.
20. The composition as defined in any preceding paragraph, wherein the curcumin compound is defined by Formula I:

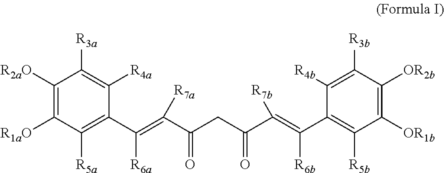

wherein:
   each R₁ₐ, R_{1b}, R₂ₐ, and R_{2b} group is independently selected from any R_{OX} group, and/or either or both pairs of R₁ₐ/R₂ₐ and/or R_{1b}/R_{2b} are linked together to form an optionally substituted heterocyclic or heteroaryl ring; and
   each R₃ₐ, R_{3b}, R₄ₐ, R_{4b}, R₅ₐ, R_{5b}, R₆ₐ, R_{6b}, R₇ₐ, and R_{7b} group is independently selected from any R_{CAR} group, and/or either or both pairs of R₃ₐ/R₄ₐ and/or R_{3b}/R_{4b} are linked together to form an optionally substituted cycloalkyl, cycloalkenyl, heterocyclic, or heteroaryl ring;
wherein:
   each R_{OX} group is selected from hydrogen, trifluoromethyl, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N,N-di-[(1-6C)alkyl]sulphamoyl, or from a group of the formula:

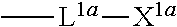

wherein:
L^{1a} is absent or is selected from SO, SO₂, CO, CH(OR_{A}), CON(R_{A}), N(R_{A})CO, SO₂N(R_{A}), wherein R_{A} is hydrogen or (1-8C)alkyl; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{OX} group is optionally further substituted with one or more R_{CAR} groups as defined below; each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

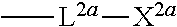

wherein:
L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above;
wherein optionally either or both OR₁ₐ and/or OR_{1b} are replaced by hydrogen (e.g. dealkoxy analogues);
or any pharmaceutically acceptable salt thereof.
21. The composition as defined in any preceding paragraph, wherein the curcumin compound is curcumin.
22. The composition as defined in any preceding paragraph, wherein the composition is free of turmerones or comprises no more than 1 wt % turmerones.
23. The composition as defined in any preceding paragraph, wherein the composition comprises bile salts (and/or bile acid(s)).
24. The composition as defined in any preceding paragraph, wherein the composition comprises one or more oils.
25. The composition as defined in any preceding paragraph, wherein the weight ratio of unstipulated lipid(s) (i.e. lipid(s) other than the one or more glyceride(s) stipulated in any preceding paragraph) to stipulated glyceride(s) (i.e. the one or more glycerides stipulated in any preceding paragraph) is between 0:100 to 15:85 (i.e. the weight ratio is less than or equal to 15:85).
26. The composition as defined in any preceding paragraph, wherein the composition is a topical composition.
27. The composition as defined in paragraph 26, wherein topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.
28. The composition as defined in any of paragraphs 1 to 25, wherein the composition is an ingestible composition.
29. The composition as defined in paragraph 28, wherein the ingestible composition is (or is incorporated within a product which is) selected from a lozenge, a gel, a jelly, a pastille, a tablet, a capsule (e.g. a capsule containing the composition), a toffee, a nougat, a chewy candy, and/or a chewing gum.
30. A composition as defined in any preceding paragraph for use as a medicament.
31. A composition as defined in any of paragraphs 1 to 29 for use in treating neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukemia, and colorectal cancer), anaphylaxis, arthritis, osteoarthritis, acne, and/or eczema.

1. A composition comprising a curcumin compound and one or more monoglycerides, wherein at least one of the one or more monoglyceride(s) comprises at least one cis-alkene moiety;
on the proviso that the composition is not a food supplement, food additive, or a foodstuff.

2. The composition as claimed in claim 1, wherein the one or more monoglycerides are one or more monoglycerides bearing a C₈-C₂₀ fatty acid moiety having at least one cis-alkene.

3. The composition as claimed in claim 2, wherein the one or more monoglycerides are a first monoglyceride and a second monoglyceride, each bearing a C₈-C₂₀ fatty acid moiety having at least one cis-alkene.

4. The composition as claimed in claim 3, wherein the one or more monoglycerides are glyceryl monooleate and glyceryl monolinoleate.

5. The composition as claimed in claim 4, wherein the glyceryl monooleate and glyceryl monolinoleate are present in a weight ratio between 10:90 and 90:10.

6. The composition as claimed in claim 1, wherein the composition comprises 0.01-40 wt % curcumin compound.

7. The composition as claimed in claim 1, wherein the composition comprises 0.1-30 wt % curcumin compound.

8. The composition as claimed in claim 1, wherein the weight ratio of the curcumin compound to the monoglyceride(s) is between 0.5:1000 and 800:1000.

9. The composition as claimed in claim 8, wherein the weight ratio of the curcumin compound to the monoglyceride(s) is between 30:1000 and 500:1000.

10. The composition as claimed in claim 7, wherein the weight ratio of the curcumin compound to the monoglyceride(s) is between 200:1000 and 600:1000.

11. The composition as claimed in claim 1, wherein the stipulated monoglyceride(s) constitute at least 85 wt % of the total amount of lipids present within the composition.

12. The composition as claimed in claim 1, wherein the composition comprises less than or equal to 60 wt % water.

13. The composition as claimed in claim 12, wherein the composition comprises 10-60 wt % water.

14. The composition as claimed in claim 12, wherein the composition has at most 2 wt % water.

15. The composition as claimed in claim 1, wherein at least 50 wt % of the composition is constituted by the stipulated ingredients (curcumin compound, monoglyceride(s), any water that is present, and any additionally stipulated ingredients).

16. The composition as claimed in claim 1, wherein centrifugation (e.g. 3000 rpm for 5 min at RT) of the composition yields less than 2 wt % sedimentation (i.e. less than 2 wt % of the composition as a whole).

17. The composition as claimed in claim 1, wherein the composition is either free of poloxamer, polysorbate, and PEG-40 castor oil surfactants or comprises at most 1 wt % free of poloxamer, polysorbate, and PEG-40 castor oil surfactants.

18. The composition as claimed in claim 17, wherein the composition is either free of surfactants or comprises at most 1 wt % surfactant(s).

19. The composition as claimed in claim 1, wherein the composition is free of any compounds that comprise polyalkylene glycol ester moieties (e.g. polyethylene glycol and/or polypropylene glycol ester moieties), and suitably also free of polyvinyl alcohol.

20. The composition as claimed in claim 1, wherein the curcumin compound is defined by Formula I: 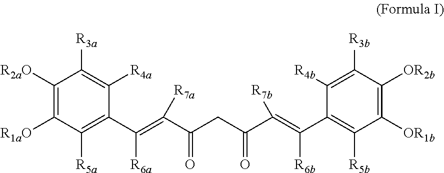
wherein:
each R₁ₐ, R_{1b}, R₂ₐ, and R_{2b} group is independently selected from any R_{OX} group, and/or either or both pairs of R₁ₐ/R₂ₐ and/or R_{1b}/R_{2b} are linked together to form an optionally substituted heterocyclic or heteroaryl ring; and
each R₃ₐ, R_{3b}, R₄ₐ, R_{4b}, R₅ₐ, R_{5b}, R₆ₐ, R_{6b}, R₇ₐ, and R_{7b} group is independently selected from any R_{CAR} group, and/or either or both pairs of R₃ₐ/R₄ₐ and/or R_{3b}/R_{4b} are linked together to form an optionally substituted cycloalkyl, cycloalkenyl, heterocyclic, or heteroaryl ring;
wherein:
each R_{OX} group is selected from hydrogen, trifluoromethyl, formyl, carboxy, carbamoyl, (1-8C)alkyl, (2-8C)alkenyl, (2-8C)alkynyl, (1-8C)hydroxyalkyl, (1-6C)alkylsulphinyl, (1-6C)alkylsulphonyl, (1-6C)alkoxycarbonyl, N-(1-6C)alkylcarbamoyl, N,N-di-[(1-6C)alkyl]carbamoyl, (2-6C)alkanoyl, N,N-di-[(1-6C)alkyl]sulphamoyl, or from a group of the formula: 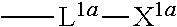
wherein:
L^{1a} is absent or is selected from SO, SO₂, CO, CH(OR_{A}), CON(R_{A}), N(R_{A})CO, SO₂N(R_{A}), wherein R_{A} is hydrogen or (1-8C)alkyl; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{OX} group is optionally further substituted with one or more R_{CAR} groups as defined below;
each R_{CAR} group is selected from hydrogen, halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula: 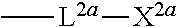
wherein:
L^{2a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein R_{B} is hydrogen or (1-8C)alkyl; and
X^{2a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any R_{CAR} group is optionally further substituted with one or more R_{CAR} groups as defined above;
wherein optionally either or both OR₁ₐ and/or OR_{1b} are replaced by hydrogen (e.g. dealkoxy analogues);
or any pharmaceutically acceptable salt thereof.

21. The composition as claimed in claim 1, wherein the curcumin compound is curcumin.

22. The composition as claimed in claim 1, wherein the composition is free of turmerones or comprises no more than 1 wt % turmerones.

23. The composition as claimed in claim 1, wherein the composition further comprises bile salts (and/or bile acid(s)).

24. The composition as claimed in claim 1, wherein the composition further comprises one or more oils.

25. The composition as claimed in claim 1, wherein the weight ratio of unstipulated lipid(s) (i.e. lipid(s) other than the one or more monoglyceride(s) stipulated in any preceding claim) to stipulated monoglyceride(s) (i.e. the one or more monoglycerides stipulated in any preceding claim) is between 0:100 to 15:85 (i.e. the weight ratio is less than or equal to 15:85).

26. The composition as claimed in claim 1, wherein the composition is a topical composition.

27. The composition as claimed in claim 26, wherein topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.

28. The composition as claimed in claim 1, wherein the composition is an ingestible composition.

29. The composition as claimed in claim 28, wherein the ingestible composition is (or is incorporated within a product which is) selected from a lozenge, a gel, a jelly, a pastille, a tablet, a capsule (e.g. a capsule containing the composition), a toffee, a nougat, a chewy candy, and/or a chewing gum.

30. The composition as claimed in claim 1, wherein the composition is a nutraceutical composition.

31. The composition as claimed in claim 1, wherein the composition is not a nutraceutical composition.

32-33. (canceled)

34. A composition comprising a curcumin compound and one or more lipids.

35. A method of treating a treating a disease, disorder or medical condition, neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukemia, and colorectal cancer), anaphylaxis, arthritis, osteoarthritis, acne, and/or eczema, comprising administering to a subject in need thereof an effective amount of the composition as claimed in claim 1.

36. The method as claimed in claim 35, wherein the disease, disorder or medical condition is a neurodegenerative disorder, inflammation, microbial infection, viral infection, fungal infection, depression, cancer, anaphylaxis, arthritis, osteoarthritis, acne, or eczema.
